(19) United States
(12) Patent Application Publication
(10) Pub. No.: US 2026/0234222 A1
(43) Pub. Date: Aug. 13, 2026

(54) Method For Prevention or Treatment of Coronavirus Infection

(71) Applicant: Leyden Laboratories B.V., Leiden (NL)

(72) Inventors: Jaap Goudsmit, Amsterdam (NL); Michael Alan Shaw, Leiden (NL)

(21) Appl. No.: 19/150,728

(22) PCT Filed: Jan. 25, 2024
(86) PCT No.: PCT/NL2024/050033

§ 371 Date: Jul. 24, 2025

(30) Foreign Application Priority Data
Jan. 25, 2023 (EP) 23153350.6
Aug. 9, 2023 (EP) 23190707.2

(51) Int. Cl. C07K 16/104; A61K 39/00; A61P 31/14

(52) U.S. Cl. CPC C07K 16/104; A61P 31/14; A61K 2039/544; C07K 2317/31; C07K 2317/522; C07K 2317/524; C07K 2317/526; C07K 2317/55; C07K 2317/565; C07K 2317/76; C07K 2317/92

(57) ABSTRACT

The invention is in the field of medical treatment and relates to a method for treating coronavirus infection. In particular, the present invention relates to methods for prophylactic and/or therapeutic treatment of coronavirus infection by means of the mucosal administration of antibodies against coronavirus.

### CROSS REFERENCE TO RELATED APPLICATIONS

This application is a national phase entry under 35 U.S.C. § 371 of International Patent Application PCT/NL2024/050033, filed Jan. 25, 2024, designating the United States of America and published in English as International Patent Publication WO2024/158285 on Aug. 2, 2024, which claims the benefit under Article 8 of the Patent Cooperation Treaty to European Patent Application Serial No. 23153350.6, filed Jan. 25, 2023, and to European Patent Application Serial No. 23190707.2, filed Aug. 9, 2023, the entireties of which are hereby incorporated by reference.

### INCORPORATION BY REFERENCE

The ST.26 XML Sequence listing named “11026-US 2025 Jul. 24-Sequence Listing ST26”, created on Feb. 28, 2024, and having a size of 602, 112 bytes, is hereby incorporated herein by this reference in its entirety.

### FIELD OF THE INVENTION

The invention is in the field of medical treatment and relates to a method for treating ß-coronavirus infections in animals and humans, including Severe Acute Respiratory Syndrome Coronavirus 2 (SARS-COV-2).

In particular, the present invention relates to methods for prophylactic and/or therapeutic treatment of SARS-COV-2 by means of mucosal administration, especially intranasal and/or oral inhalation of antibodies against β-coronavirus, in particular SARS-COV-2.

### BACKGROUND OF THE INVENTION

The SARS-COV-2 virus causes the disease COVID-19 in humans and is widespread having a significant impact on human society. We should expect the SARS-COV-2 virus and its variants of concern to continue to cause frequent infections, as well as severe disease and death. As of January 2023, the ongoing COVID-19 pandemic caused by SARS-COV-2, has resulted in more than 6.7 million worldwide deaths since 2019.

In recent years, outbreaks of other coronaviruses have caused severe illness and deaths. As scientists develop therapeutic antibodies and vaccines against SARS-COV-2, the danger remains of future novel variants of SARS-COV-2 and novel coronaviruses emerging. These novel coronaviruses can lead to morbidity, mortality, and could cause pandemics. It remains vitally important to identify broadly protective therapies that can combat current and emerging coronaviruses in the future.

In addition to SARS-COV-2, four other betacoronaviruses are known to cause disease in humans: the betacoronaviruses HCoV-OC43 (human coronavirus OC43), HCoV-HKU1 (human coronavirus HKU1), SARS-COV (severe acute respiratory syndrome coronavirus) and MERS-COV (Middle East respiratory syndrome coronavirus). Spillover events, where humans get infected with coronaviruses that circulate in animal reservoirs, are common. Recently, three betacoronaviruses crossed over from animals to humans, including SARS-CoV-2 causing serious outbreaks.

Furthermore, two coronaviruses previously linked only to animal infection were recently detected in humans who presented with flu-like symptoms.

Coronaviruses derive their name from their crown-like appearance. Coronaviruses are a large group of viruses that have spike proteins on their surface, resembling crown-like thorns. The antibodies elicited by natural infection or by COVID-19 vaccines used for mass immunization between 2020-2023 primarily target the variable receptor binding domain (RBD) of these spike proteins.

### Structure of the Spike Protein

Coronavirus infection is a multistep process that involves enzymatic cleavage and rearrangement of the surface spike protein. The spike protein has an S1 subunit with the receptor-binding domain (RBD) and an S2 subunit involved in fusion of the viral and (host) cell membranes thereby facilitating cell entry.

The viral spike protein of SARS-COV-2 facilitates viral entry by binding primarily to the angiotensin-converting enzyme 2 (ACE2) receptor on host cells.

The SARS-COV-2 spike contains two cleavage sites: a furin cleavage site at the boundary of the S1 and S2 subunits, and an S2′ site that is highly conserved among different coronaviruses.

### S1 Subunit

The SARS-COV-2 spike protein uses the RBD on the S1 subunit to engage the target cell's ACE2 receptor.

The S1 subunit is more accessible and remains the main target of many neutralizing antibodies. The S1 subunit, however, is more genetically variable than the S2 subunit; especially when subjected to selective pressure from antibodies. This propensity towards genetic variability can lead to viral variants with the predominant changes occurring on the S1 subunit. Changes in the receptor-binding domain in SARS-COV-2 variants of concern have resulted in frequent reinfections, as protection by the antibodies elicited by previous infection and/or vaccines dramatically reduced.

### S2 Subunit

The viral spike components essential to infection also involve the structurally complex S2 subunit. The S2 subunit contains dynamic elements essential for fusion with the host cell. Once the receptor has bound, the S1 subunit is discarded and the membrane enzyme transmembrane serine protease 2 (TMPRSS2) or endosomal cathepsins cleave the S2 site.

This cleaving leads to insertion of the fusion peptide into the cell membrane culminating in viral fusion.

These S2 subunit elements are less disposed towards genetic variability than the RBD, which so far has been capable of retaining or even increasing binding capabilities to ACE2 despite a variety of mutations.

The S2 domain sites yield poorly accessible targets for novel therapeutics to protect against a wider range of coronaviruses.

### The Stem Helix

The stem helix at the base of the viral spike protein is even more difficult to access than elements on the S2 subunit but benefits from better-preserved amino acid sequencing.

### Treatments and Prevention

Limited options for the viral treatment of SARS-COV-2 exist in the form of small molecule or antibody antiviral drugs. For prevention of SARS-COV-2 infection, few interventions exist. Most are non-pharmaceutical interventions such as social distancing and mask wearing. These methods are non-specific, and efficacy largely depends on compliance. Thus, there is a need for different treatments, especially treatments that simultaneously target the multitude of SARS-COV-2 variants of concern such as, for example, broad-neutralizing antibodies (bnAbs).

Although treatment of SARS-COV-2 infection with one, two or more antibodies is a possibility, a treatment comprising a single antibody or combination of antibodies would be considerably more cost-efficient. The currently dominant SARS-COV-2 Omicron subvariant BA.5 is resistant to most monoclonal antibody therapeutics. Betacoronaviruses, including SARS-COV-2, also infect a range of species know to have frequent human contact, thus increasing the risk of zoonotic transfer and new outbreaks with SARS-COV-2 variants or viruses that previously have not infected humans and for which no therapies exist.

There is therefore an urgent need to develop therapeutic mAbs that broadly target β-coronaviruses, as well as new SARS-COV-2 variants of concern that are not neutralized by antibodies elicited by immunization and/or natural infection.

It remains vitally important to identify broadly protective therapies that can combat novel coronaviruses. We have surprisingly developed a novel array of broadly protective antibodies which are disclosed herein.

### SUMMARY OF THE INVENTION

The inventors have discovered that an antibody having complementarity-determining regions (CDR) as disclosed herein can advantageously be used in the treatment of a coronavirus infection, especially in the prophylactic treatment of coronavirus infection.

The inventors have surprisingly established when treating a coronavirus infection, mucosal administration of an antibody having CDRs as disclosed herein is a more potent prophylactic route of administration method compared with intraperitoneal administration.

The inventors have surprisingly established when treating a coronavirus infection, intranasal administration of an antibody having CDRs as disclosed herein is a more potent prophylactic route of administration method compared with intraperitoneal administration.

Significantly, the mucosal administration method of treatment of the present invention is effective against SARS-Cov-2 and variants of concern and does not require the use of additional antibodies.

A mucosal administration treatment method of the invention is effective even at low dosages and prevents body weight loss.

### Complementarity-Determining Regions (CDRs)

Preferably, CDR regions are according to Kabat et al., (1991) as described in Sequences of Proteins of Immunological Interest.

In a preferred embodiment, the invention provides an antibody that comprises a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574.

In a preferred embodiment, the invention provides an antibody comprising a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574, wherein the antibody is administered to mucosal epithelium.

In a preferred embodiment, the invention provides a method for the treatment of a coronavirus infection in an individual, the method comprising administering to an individual in need thereof an antibody comprising a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574, wherein the antibody is administered to mucosal epithelium.

In a preferred embodiment, the invention provides a mucosal composition comprising an antibody comprising a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574.

In a preferred embodiment, the invention provides an antibody for use in a method for treatment of a coronavirus infection in an individual, wherein the antibody comprises a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574, wherein the antibody is administered to mucosal epithelium.

In a preferred embodiment, the invention provides an antibody, or a method or composition comprising the antibody, wherein the antibody comprises a heavy chain variable domain that comprises a heavy chain CDR1 region comprising SEQ ID NO: 055, a heavy chain CDR2 region comprising SEQ ID NO: 137, and a heavy chain CDR3 region comprising SEQ ID NO: 257, a light chain variable domain that comprises a light chain CDR1 region comprising SEQ ID NO: 363, a light chain CDR2 region comprising SEQ ID NO: 480, and a light chain CDR3 region comprising SEQ ID NO: 574.

In a preferred embodiment, an antibody is disclosed herein for use in a method for the prophylactic treatment of a coronavirus infection, in an individual, the antibody comprising a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574, and wherein between 0.1 mg and 20 mg of the antibody is administered to the mucosa.

In a preferred embodiment, an antibody is disclosed herein for use in a method for the prophylactic treatment of a coronavirus infection, in an individual, the antibody comprising a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574, and wherein between 0.1 mg and 20 mg of the antibody is administered intranasally.

In a preferred embodiment, an antibody is disclosed herein for use in a method for the prophylactic treatment of a coronavirus infection, in an individual, the antibody comprising a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574, and wherein between 0.1 mg and 20 mg of the antibody is administered by oral inhalation.

In a preferred embodiment, a composition comprising an antibody is disclosed herein, the antibody comprising a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574, for use in a method of prevention or treatment of a coronavirus infection, wherein the administration route comprises at least one of pulmonary administration, nasal administration and oropharyngeal administration and wherein the nominal dose of the antibody is between 0.1 mg and 20 mg.

Preferably, an antibody as disclosed herein comprises a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257.

Preferably, an antibody as disclosed herein comprises a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574.

Preferably, in a method for treatment of the invention, the antibody comprises a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257.

Preferably, in a method for treatment of the invention, the antibody comprises a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574.

In an alternative preferred embodiment, the invention provides an antibody that comprises a heavy chain variable domain that comprises a heavy chain CDR1 region comprising any one of SEQ ID NO: 001 to 006, 012 to 017, 023 to 028, 033 to 038, 042 to 047, 050 to 054, a heavy chain CDR2 region comprising any one of SEQ ID NO: 077 to 082, 088 to 093, 099 to 104, 110 to 115, 121 to 126, 132 to 136, and a heavy chain CDR3 region comprising any one of SEQ ID NO: 197 to 202, 208 to 213, 219 to 224, 230 to 235, 241 to 246, 252 to 256, a light chain variable domain that comprises a light chain CDR1 region comprising any one of SEQ ID NO: 303 to 308, 314 to 319, 325 to 330, 336 to 341, 347 to 352, 358 to 362, a light chain CDR2 region comprising any one of SEQ ID NO: 421 to 426, 432 to 437, 443 to 448, 454 to 459, 465 to 470, 475 to 479, and a light chain CDR3 region comprising any one of SEQ ID NO: 514 to 519, 525 to 530, 536 to 541, 547 to 552, 558 to 563, 569 to 573.

### Embodiments Having >1 CDRs

Preferably, an antibody as disclosed herein comprises a heavy chain variable domain that comprises a heavy chain CDR2 region comprising any one of SEQ ID NO: 077 to 082, 088 to 093, 099 to 104, 110 to 115, 121 to 126, 132 to 137.

### Route of Administration

In a preferred embodiment, the invention provides an antibody as disclosed herein, or a method or composition comprising the antibody as disclosed herein, wherein the antibody is administered by mucosal administration.

In a preferred embodiment, the invention provides an antibody as disclosed herein, or a method or composition comprising the antibody as disclosed herein, wherein the antibody is administered by intravenous administration.

In a preferred embodiment, the invention provides an antibody as disclosed herein, or a method or composition comprising the antibody as disclosed herein, wherein the antibody is administered by at least one of oral inhalation, nasal administration, ocular administration, vaginal administration, rectal administration and oropharyngeal administration.

In a preferred embodiment, the antibody as disclosed herein is administered intranasally.

Such an antibody is useful for the treatment of coronavirus infection in an individual. Preferably, the method for treatment of coronavirus infection is a method for prophylactic and/or therapeutic treatment of a coronavirus infection. Preferably, the antibody is provided to an individual infected with coronavirus. Preferably, the antibody is provided to the individual prophylactically. Preferably, the antibody is provided to the individual after infection but prior to onset of symptoms.

In a preferred embodiment, the disclosure provides a method of treating coronavirus infection in an individual, said method comprising mucosal administration, preferably administering intranasally and/or by oral inhalation, to an individual in need thereof an antibody comprising a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574.

In a preferred embodiment, the individual is infected with coronavirus or at risk of coronavirus infection.

In a preferred embodiment, the disclosure provides an antibody as disclosed herein for use in the manufacture of a medicament for use in the treatment of a coronavirus infection.

### CLAUSES

The following numbered clauses each represent preferred embodiments of the invention and are part of the description.

### Clause 1:

A method for the treatment of a coronavirus infection in an individual, the method comprising administering to an individual in need thereof an antibody comprising a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574, wherein the antibody is administered to the mucosa.

### Clause 2:

A mucosal composition comprising an antibody comprising a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574.

### Clause 3:

An antibody for use in a method of prevention or treatment of a coronavirus infection in an individual, wherein the antibody comprises a heavy chain variable domain that comprises a heavy chain CDR1 region comprising any one of SEQ ID NO: 001 to 006, 012 to 017, 023 to 028, 033 to 038, 042 to 047, 050 to 055, a heavy chain CDR2 region comprising any one of SEQ ID NO: 077 to 082, 088 to 093, 099 to 104, 110 to 115, 121 to 126, 132 to 137, and a heavy chain CDR3 region comprising any one of SEQ ID NO: 197 to 202, 208 to 213, 219 to 224, 230 to 235, 241 to 246, 252 to 257, a light chain variable domain that comprises a light chain CDR1 region comprising any one of SEQ ID NO: 303 to 308, 314 to 319, 325 to 330, 336 to 341, 347 to 352, 358 to 363, a light chain CDR2 region comprising any one of SEQ ID NO: 421 to 426, 432 to 437, 443 to 448, 454 to 459, 465 to 470, 475 to 480, and a light chain CDR3 region comprising any one of SEQ ID NO: 514 to 519, 525 to 530, 536 to 541, 547 to 552, 558 to 563, 569 to 574, wherein the antibody is administered to mucosa.

### Clause 4:

A method, composition or antibody according to any preceding Clause, wherein the antibody comprises a heavy chain variable domain that comprises a heavy chain CDR1 region comprising SEQ ID NO: 055, a heavy chain CDR2 region comprising SEQ ID NO: 137, and a heavy chain CDR3 region comprising SEQ ID NO: 257, a light chain variable domain that comprises a light chain CDR1 region comprising SEQ ID NO: 363, a light chain CDR2 region comprising SEQ ID NO: 480, and a light chain CDR3 region comprising SEQ ID NO: 574.

### Clause 5:

A method, composition or antibody according to any preceding Clause, wherein the method for treatment of coronavirus virus infection is a method for prophylactic and/or therapeutic treatment of a coronavirus, preferably wherein the method for treatment is for the prophylactic treatment of a SARS-COV-2 infection.

### Clause 6:

A method, composition or antibody according to any preceding Clause, wherein the antibody comprises a heavy chain variable domain having the sequence of SEQ ID NO:629 having at most 15, preferably 0, 1, 2, 3, 4, 5, 6, 7, 8, 9 or 10 amino acid insertions, deletions, substitutions which are not in the heavy chain CDRs.

### Clause 7:

A method, composition or antibody according to any preceding Clause, wherein the antibody comprises a light chain variable domain having the sequence of SEQ ID NO:630 having at most 15, preferably 0, 1, 2, 3, 4, 5, 6, 7, 8, 9 or 10 amino acid insertions, deletions, substitutions which are not in the light chain CDRs.

### Clause 8:

A method, composition or antibody according to any preceding Clause, wherein the antibody is an IgG antibody, preferably an IgG1 antibody.

### Clause 9:

A method, composition or antibody according to any preceding Clause, wherein the antibody is provided to the individual prophylactically.

### Clause 10:

A method, composition or antibody according to any preceding Clause, wherein the administration route comprises at least one of oral inhalation, nasal administration, ocular administration and oropharyngeal administration.

### Clause 11:

A method, composition or antibody according to any preceding Clause, wherein the antibody is administered at least once or at least twice monthly.

### Clause 12:

A method, composition or antibody according to any preceding Clause, wherein the antibody is administered to the individual at a dosage of between 0.01 mg and 20 mg.

### Clause 13:

A composition according to any preceding Clause, comprising an antibody in a single dose unit of between 0.01 mg and 20 mg, preferably between 0.1 mg and 15 mg or preferably 0.5 mg and 10 mg, wherein the antibody is as defined in any one of Clauses 1 to 12.

### Clause 14:

A composition according to any preceding Clause, wherein the composition is self-administered.

### Clause 15:

A medicament delivery device comprising a composition according to any preceding Clause.

### Dosing

Preferably, between 0.01 mg and 20 mg of the antibody is administered to an individual, more preferably between 0.1 mg and 20 mg of the antibody is administered to an individual.

The flat dose, or nominal dose, of the antibody is preferably between 0.01 mg and 17 mg, preferably between 0.011 mg and 16 mg, preferably between 0.012 mg and 15 mg, preferably between 0.013 mg and 14 mg, preferably between 0.014 mg and 13 mg, preferably between 0.015 mg and 12 mg, preferably between 0.016 mg and 11 mg, preferably between 0.017 mg and 10 mg, preferably between 0.018 mg and 9 mg, preferably between 0.020 mg and 8 mg, preferably between 0.023 mg and 7 mg, preferably between 0.025 mg and 6 mg, preferably between 0.030 mg and 5 mg, preferably between 0.040 mg and 4 mg, preferably between 0.050 mg and 3 mg, preferably between 0.075 mg and 2 mg, or preferably between 0.10 mg and 1 mg.

The flat dose, or nominal dose, of the antibody is preferably between 0.010 mg and 5.0 mg, preferably between 0.020 mg and 4.5 mg, preferably between 0.030 mg and 4.0 mg, preferably between 0.040 mg and 3.5 mg, preferably between 0.050 mg and 3.0 mg, preferably between 0.060 mg and 2.5 mg, preferably between 0.070 mg and 2.0 mg, preferably between 0.080 mg and 1.5 mg, preferably between 0.090 mg and 1.0 mg, or preferably between 0.100 mg and 0.5 mg.

The flat dose, or nominal dose, of the antibody is preferably between 0.010 mg and 15.0 mg, preferably between 0.020 mg and 14.5 mg, preferably between 0.030 mg and 4.0 mg, preferably between 0.040 mg and 13.5 mg, preferably between 0.050 mg and 13.0 mg, preferably between 0.060 mg and 12.5 mg, preferably between 0.070 mg and 12.0 mg, preferably between 0.080 mg and 11.5 mg, preferably between 0.090 mg and 11.0 mg, or preferably between 0.100 mg and 10.5 mg.

The flat dose, or nominal dose, of the antibody to be delivered to a human subject is preferably between 0.1 μg and 10 μg, preferably between 0.2 μg and 9 μg, preferably between 0.3 μg and 8 μg, preferably between 0.4 μg and 7 μg, preferably between 0.5 μg and 6 μg, preferably between 0.6 μg and 5 μg, preferably between 0.7 μg and 4 μg, preferably between 0.8 μg and 3 μg, preferably between 0.9 μg and 2 μg or preferably around 1 μg.

The flat dose, or nominal dose, of the antibody to be delivered to a human subject is preferably between 1 mg and 11 mg, preferably between 2 mg and 12 mg, preferably between 3 mg and 13 mg, preferably between 4 mg and 14 mg, preferably between 5 mg and 15 mg, preferably between 6 mg and 16 mg, preferably between 7 mg and 17 mg, preferably between 8 mg and 18 mg, preferably between 9 mg and 19 mg, or preferably between 10 mg and 20 mg.

The flat dose, or nominal dose, of the antibody to be delivered to a human subject is preferably between 1 mg and 20 mg, preferably between 2 mg and 19 mg, preferably between 3 mg and 18 mg, preferably between 4 mg and 17 mg, preferably between 5 mg and 16 mg, preferably between 6 mg and 15 mg, preferably between 7 mg and 14 mg, preferably between 8 mg and 13 mg, preferably between 9 mg and 12 mg, or preferably between 10 mg and 11 mg.

The flat dose, or nominal dose, of the antibody to be delivered to a human subject is preferably between 11 mg and 20 mg, preferably between 10 mg and 19 mg, preferably between 9 mg and 18 mg, preferably between 8 mg and 17 mg, preferably between 7 mg and 16 mg, preferably between 6 mg and 15 mg, preferably between 5 mg and 14 mg, preferably between 4 mg and 13 mg, preferably between 3 mg and 12 mg, preferably between 2 mg and 11 mg, or preferably between 1 mg and 10 mg.

In a preferred embodiment, the disclosure further provides a composition formulated for mucosal administration comprising an antibody as disclosed herein in a single dose unit of between 0.1 mg and 20 mg, preferably between 5 mg and 15 mg or preferably 7.5 mg and 12.5 mg.

In a preferred embodiment, the disclosure further provides a composition formulated for intranasal administration comprising an antibody as disclosed herein in a single dose unit of between 0.1 mg and 20 mg, preferably between 5 mg and 15 mg or preferably 7.5 mg and 12.5 mg.

In a preferred embodiment, the disclosure further provides a composition formulated for oral inhalation comprising an antibody as disclosed herein in a single dose unit of between 0.1 mg and 20 mg, preferably between 5 mg and 15 mg or preferably 7.5 mg and 12.5 mg.

### Timing and Intervals

Preferably, the antibody is administered at least once or at least twice per month.

Preferably, the antibody is administered at least once or at least twice per week.

The weekly flat dose, or nominal dose, of the antibody is between 0.010 mg and 17 mg, preferably between 0.011 mg and 16 mg, preferably between 0.012 mg and 15 mg, preferably between 0.013 mg and 14 mg, preferably between 0.014 mg and 13 mg, preferably between 0.015 mg and 12 mg, preferably between 0.016 mg and 11 mg, preferably between 0.017 mg and 10 mg, preferably between 0.018 mg and 9 mg, preferably between 0.020 mg and 8 mg, preferably between 0.023 mg and 7 mg, preferably between 0.025 mg and 6 mg, preferably between 0.030 mg and 5 mg, preferably between 0.040 mg and 4 mg, preferably between 0.050 mg and 3 mg, preferably between 0.075 mg and 2 mg, or preferably between 0.10 mg and 1 mg.

The weekly flat dose, or nominal dose, of the antibody is between 0.010 mg and 5.0 mg, preferably between 0.020 mg and 4.5 mg, preferably between 0.030 mg and 4.0 mg, preferably between 0.040 mg and 3.5 mg, preferably between 0.050 mg and 3.0 mg, preferably between 0.060 mg and 2.5 mg, preferably between 0.070 mg and 2.0 mg, preferably between 0.080 mg and 1.5 mg, preferably between 0.090 mg and 1.0 mg, or preferably between 0.100 mg and 0.5 mg.

Preferably, the antibody is administered at least once or at least twice per day. Preferably, the antibody is administered daily. Preferably, the antibody is administered twice daily.

Preferably, the daily flat dose, or nominal dose, is between 0.001 mg and 17 mg, preferably between 0.011 mg and 16 mg, preferably between 0.012 mg and 15 mg, preferably between 0.013 mg and 14 mg, preferably between 0.014 mg and 13 mg, preferably between 0.015 mg and 12 mg, preferably between 0.016 mg and 11 mg, preferably between 0.017 mg and 10 mg, preferably between 0.018 mg and 9 mg, preferably between 0.020 mg and 8 mg, preferably between 0.023 mg and 7 mg, preferably between 0.025 mg and 6 mg, preferably between 0.030 mg and 5 mg, preferably between 0.040 mg and 4 mg, preferably between 0.050 mg and 3 mg, preferably between 0.075 mg and 2 mg, or preferably between 0.10 mg and 1 mg.

### Framework Regions

Preferably, the antibody comprises a heavy chain variable domain having the sequence of SEQ ID NO: 629 and/or a light chain variable domain having the sequence of SEQ ID NO: 630.

Preferably, the heavy chain variable domain of said antibody further comprises a heavy chain framework region FR1 of SEQ ID NO: 631, a heavy chain framework region FR2 of SEQ ID NO: 632, a heavy chain framework region FR3 of SEQ ID NO: 633, and/or a heavy chain framework region FR4 of SEQ ID NO: 634.

Preferably, said light chain variable domain further comprises a light chain framework region FR1 of SEQ ID NO: 635, a light chain framework region FR2 of SEQ ID NO: 636, a light chain framework region FR3 of SEQ ID NO: 637, and/or a light chain framework region FR4 of SEQ ID NO: 638.

### Combination Compositions

In a preferred embodiment, we disclose a composition comprising a First binding fragment as disclosed herein and a Second binding fragment, wherein the First fragment comprises a sequence comprising any one of SEQ ID NO: 001 to 006, 012 to 017, 023 to 028, 033 to 038, 042 to 047, 050 to 055, 077 to 082, 088 to 093, 099 to 104, 110 to 115, 121 to 126, 132 to 137, 197 to 202, 208 to 213, 219 to 224, 230 to 235, 241 to 246, 252 to 257, 303 to 308, 314 to 319, 325 to 330, 336 to 341, 347 to 352, 358 to 363, 421 to 426, 432 to 437, 443 to 448, 454 to 459, 465 to 470, 475 to 480, 514 to 519, 525 to 530, 536 to 541, 547 to 552, 558 to 563, 569 to 574. More preferably wherein the First fragment comprises a sequence comprising any one or more of SEQ ID NO: 055, 137, 257, 363, 480 or 574. The Second binding fragment comprises a sequence comprising any one of SEQ ID NO.: 639, 640, 641, 642, 643 or SEQ ID NO.: 644. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose a composition comprising a First binding fragment as disclosed herein and a Second binding fragment, wherein the First fragment comprises a sequence comprising any one of SEQ ID NO: 001 to 006, 012 to 017, 023 to 028, 033 to 038, 042 to 047, 050 to 055, 077 to 082, 088 to 093, 099 to 104, 110 to 115, 121 to 126, 132 to 137, 197 to 202, 208 to 213, 219 to 224, 230 to 235, 241 to 246, 252 to 257, 303 to 308, 314 to 319, 325 to 330, 336 to 341, 347 to 352, 358 to 363, 421 to 426, 432 to 437, 443 to 448, 454 to 459, 465 to 470, 475 to 480, 514 to 519, 525 to 530, 536 to 541, 547 to 552, 558 to 563, 569 to 574. More preferably wherein the First fragment comprises any one or more of SEQ ID NO: 055, 137, 257, 363, 480 or 574. The Second binding fragment comprises a sequence comprising any one of SEQ ID NO.: 645, 646, 647, 648, 649 or SEQ ID NO.: 650. Preferably, the fragment is selected from anyone of the group comprising a full-length antibody, a Fab, modified Fab, Fab′, modified Fab′, F(ab′) 2, Fv, single domain antibodies, scFv, ScFV-Fc, bi, tri or tetra-valent antibodies, Bis-scFv, diabodies, triabodies, tetrabodies and epitope binding fragments thereof.

In a preferred embodiment, we disclose a composition comprising a First binding fragment as disclosed herein and a Second binding fragment, wherein the First fragment comprises a sequence comprising any one of SEQ ID NO: 001 to 006, 012 to 017, 023 to 028, 033 to 038, 042 to 047, 050 to 055, 077 to 082, 088 to 093, 099 to 104, 110 to 115, 121 to 126, 132 to 137, 197 to 202, 208 to 213, 219 to 224, 230 to 235, 241 to 246, 252 to 257, 303 to 308, 314 to 319, 325 to 330, 336 to 341, 347 to 352, 358 to 363, 421 to 426, 432 to 437, 443 to 448, 454 to 459, 465 to 470, 475 to 480, 514 to 519, 525 to 530, 536 to 541, 547 to 552, 558 to 563, 569 to 574. More preferably wherein the First fragment comprises a sequence comprising any one or more of SEQ ID NO: 055, 137, 257, 363, 480 or 574. The Second binding fragment comprises a sequence comprising any one of SEQ ID NO.: 651, 652, 653, 654, 655 or SEQ ID NO.: 656. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose a composition comprising a First binding fragment as disclosed herein and a Second binding fragment, wherein the First fragment comprises a sequence comprising any one of SEQ ID NO: 001 to 006, 012 to 017, 023 to 028, 033 to 038, 042 to 047, 050 to 055, 077 to 082, 088 to 093, 099 to 104, 110 to 115, 121 to 126, 132 to 137, 197 to 202, 208 to 213, 219 to 224, 230 to 235, 241 to 246, 252 to 257, 303 to 308, 314 to 319, 325 to 330, 336 to 341, 347 to 352, 358 to 363, 421 to 426, 432 to 437, 443 to 448, 454 to 459, 465 to 470, 475 to 480, 514 to 519, 525 to 530, 536 to 541, 547 to 552, 558 to 563, 569 to 574. More preferably wherein the First fragment comprises a sequence comprising any one or more of SEQ ID NO: 055, 137, 257, 363, 480 and 574. The Second binding fragment comprises a sequence comprising any one of SEQ ID NO.: 657, 658, 659, 660, 661 or SEQ ID NO.: 662. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

### Bispecific Antibodies

In a preferred embodiment, we disclose a bispecific antibody, wherein the bispecific antibody is capable of binding to the stem helix of a coronavirus. In a preferred embodiment, we disclose a bispecific antibody, wherein the bispecific antibody is capable of binding to the stem helix of SARS-COV-2.

In a preferred embodiment, we disclose an anti-coronavirus bispecific antibody or an antigen binding fragment thereof, having a first Fab capable of binding to the stem helix of a coronavirus and a second Fab capable of binding to the fusion peptide of a coronavirus.

In a preferred embodiment, we disclose an anti-SARS-COV-2 bispecific antibody or an antigen binding fragment thereof, having a first Fab capable of binding to the stem helix of SARS-COV-2 and a second Fab capable of binding to the fusion peptide of SARS-COV-2.

In a preferred embodiment, we disclose an anti-SARS-COV-2 bispecific antibody or an antigen binding fragment thereof, having a first Fab capable of binding to the stem helix of SARS-COV-2 and a second Fab capable of binding to the fusion peptide of SARS-COV-2, wherein the first Fab comprises a sequence comprising any one or more of preferred SEQ ID Numbers: 055, 137, 257, 363, 480 or 574; alternatively wherein the first Fab comprises a sequence comprising any one or more of preferred SEQ ID Numbers: 651, 652, 653, 654, 655 or 656; or alternatively wherein the first Fab comprises a sequence comprising any one or more of preferred SEQ ID Numbers: 657, 658, 659, 660, 661 to 662, and wherein the second Fab comprises a sequence comprising any one or more of preferred SEQ ID Numbers: 639, 640, 641, 642, 643 to 644; or alternatively wherein the second Fab comprises a sequence comprising any one or more of preferred SEQ ID Numbers: 645, 646, 647, 648, 649 to 650.

In a preferred embodiment, we disclose an anti-SARS-COV-2 bispecific antibody or an antigen binding fragment thereof, having a first Fab capable of binding to the stem helix of SARS-COV-2 and a second Fab capable of binding to the fusion peptide of SARS-COV-2, wherein the first Fab comprises a sequence comprising any one or more of preferred SEQ ID Numbers: 055, 137, 257, 363, 480 or 574 and wherein the second Fab comprises a sequence comprising any one or more of preferred SEQ ID Numbers: 639, 640, 641, 642, 643 to 644. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose an anti-SARS-COV-2 bispecific antibody or an antigen binding fragment thereof, having a first Fab capable of binding to the stem helix of SARS-COV-2 and a second Fab capable of binding to the fusion peptide of SARS-COV-2, wherein the first Fab comprises a sequence comprising any one or more of preferred SEQ ID Numbers: 055, 137, 257, 363, 480 or 574 and wherein the second Fab comprises a sequence comprising any one or more of preferred SEQ ID Numbers: 645, 646, 647, 648, 649 to 650. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose an anti-SARS-COV-2 bispecific antibody or an antigen binding fragment thereof, having a first Fab capable of binding to the stem helix of SARS-COV-2 and a second Fab also capable of binding to the stem helix of SARS-COV-2, wherein the first Fab comprises a sequence comprising any one or more of preferred SEQ ID Numbers: 055, 137, 257, 363, 480 or 574 and wherein the second Fab comprises a sequence comprising any one or more of preferred SEQ ID Numbers: 651, 652, 653, 654, 655 to 656. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose an anti-SARS-COV-2 bispecific antibody or an antigen binding fragment thereof, having a first Fab capable of binding to the stem helix of SARS-COV-2 and a second Fab also capable of binding to the stem helix of SARS-COV-2, wherein the first Fab comprises a sequence comprising any one or more of preferred SEQ ID Numbers: 055, 137, 257, 363, 480 or 574 and wherein the second Fab comprises a sequence comprising any one or more of preferred SEQ ID Numbers: 657, 658, 659, 660, 661 to 662. Preferably, the fragment is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

In a preferred embodiment, we disclose a bispecific antibody comprising a First Fab and a Second Fab, wherein the First Fab comprises any one or more of sequences shown in preferred SEQ ID Numbers: 055, 137 or 257, and wherein the Second Fab comprises any one or more of sequences shown in preferred SEQ ID Numbers: 660, 661, or 662.

In a preferred embodiment, we disclose a bispecific antibody comprising a First Fab and a Second Fab, wherein the First Fab comprises any one or more of sequences shown in preferred SEQ ID Numbers: 055, 137 or 257, and wherein the Second Fab comprises any one or more of sequences shown in preferred SEQ ID Numbers: 642, 643, or 644.

In a preferred embodiment, we disclose a bispecific antibody comprising a First Fab and a Second Fab, wherein the First Fab comprises any one or more of sequences shown in preferred SEQ ID Numbers: 055, 137 or 257, and wherein the Second Fab comprises any one or more of sequences shown in preferred SEQ ID Numbers: 648, 649, or 650.

In a preferred embodiment, we disclose a bispecific antibody comprising a First Fab and a Second Fab, wherein the First Fab comprises any one or more of sequences shown in preferred SEQ ID Numbers: 055, 137 or 257, and wherein the Second Fab comprises any one or more of sequences shown in preferred SEQ ID Numbers: 654, 655, or 656.

In a preferred embodiment, we disclose a bispecific antibody comprising a First Fab and a Second Fab, wherein the First Fab comprises any one or more of sequences shown in preferred SEQ ID Numbers: 657, 658, or 659, and wherein the Second Fab comprises any one or more of sequences shown in preferred SEQ ID Numbers: 363, 480 or 574.

In a preferred embodiment, we disclose a bispecific antibody comprising a First Fab and a Second Fab, wherein the First Fab comprises any one or more of sequences shown in preferred SEQ ID Numbers: 639, 640, or 641, and wherein the Second Fab comprises any one or more of sequences shown in preferred SEQ ID Numbers: 363, 480 or 574.

In a preferred embodiment, we disclose a bispecific antibody comprising a First Fab and a Second Fab, wherein the First Fab comprises any one or more of sequences shown in preferred SEQ ID Numbers: 645, 646, or 647, and wherein the Second Fab comprises any one or more of sequences shown in preferred SEQ ID Numbers: 363, 480 or 574.

In a preferred embodiment, we disclose a bispecific antibody comprising a First Fab and a Second Fab, wherein the First Fab comprises any one or more of sequences shown in preferred SEQ ID Numbers: 651, 652 or 653, and wherein the Second Fab comprises any one or more of sequences shown in preferred SEQ ID Numbers: 363, 480 or 574.

The present invention also provides construction, expression, purification methods for a bispecific antibody having a function of binding to the stem helix of SARS-COV-2, and use of the bispecific antibody in the field of medicine and especially in the prevention and/or treatment of SARS-COV-2 infections.

### CLAUSES

The following numbered clauses each represent preferred embodiments of the invention and are part of the description.

### Clause 26:

An anti-coronavirus, preferably anti-SARS-COV-2 bispecific antibody or an antigen binding fragment thereof, having a first Fab capable of binding to the stem helix of a coronavirus, preferably SARS-COV-2 and a second Fab capable of binding to the fusion peptide of a coronavirus, preferably SARS-COV-2, wherein the first Fab comprises a sequence comprising any one or more of preferred SEQ ID Numbers: 055, 137, 257, 363, 480 or 574 and wherein the second Fab comprises a sequence comprising any one or more of preferred SEQ ID Numbers: 639, 640, 641, 642, 643 to 644.

### Clause 27:

A bispecific antibody or an antigen binding fragment thereof according to Clause 26, comprising a first Fab capable of binding to the stem helix of a coronavirus, preferably SARS-COV-2, and a second Fab capable of binding to the fusion peptide of a coronavirus, preferably SARS-COV-2, wherein the first Fab comprises a sequence comprising any one or more of preferred SEQ ID Numbers: 055, 137, 257, 363, 480 and/or 574 and wherein the second Fab comprises a sequence comprising any one or more of preferred SEQ ID Numbers: 639, 640, 641, 642, 643 and/or 644.

### Clause 28:

A bispecific antibody or an antigen binding fragment thereof according to Clauses 26 or 27, comprising a first Fab specifically binding to the stem helix of a coronavirus, preferably SARS-COV-2, and a second Fab specifically binding to the fusion peptide of a coronavirus, preferably SARS-COV-2, characterized in that:
a.) the first Fab comprises a heavy chain variable region comprising as CDRs, a Heavy Chain CDR1 region of SEQ ID NO: 055, a Heavy Chain CDR2 region of SEQ ID NO: 137 and a Heavy Chain CDR3 region of SEQ ID NO: 257 and a light chain variable region comprising as CDRs a Light Chain CDR1 region of SEQ ID NO: 363, a Light Chain CDR2 region of SEQ ID NO: 480 and a Light Chain CDR3 region of SEQ ID NO: 574, and
b.) the second Fab comprises a heavy chain variable region comprising as CDRs, a Heavy Chain CDR1 region of SEQ ID NO: 639, a Heavy Chain CDR2 region of SEQ ID NO: 640 and a Heavy Chain CDR3 region of SEQ ID NO: 641 and a light chain variable region comprising as CDRs a Light Chain CDR1 region of SEQ ID NO: 642, a Light Chain CDR2 region of SEQ ID NO: 643 and a Light Chain CDR3 region of SEQ ID NO: 644.

### Clause 29:

A bispecific antibody or an antigen binding fragment thereof according to any of Clauses 26 to 28, comprising:
a) A light chain comprising VL-CL domains and a heavy chain comprising VH-CH1-CH2-CH3 domains of an antibody or an antigen binding fragment thereof, capable of binding to the stem helix of a coronavirus, preferably SARS-COV-2, and
b) A light chain comprising VL-CL domains and a heavy chain comprising VH-CH1-CH2-CH3 domains of an antibody or an antigen binding fragment thereof, capable of binding to the fusion peptide of a coronavirus, preferably SARS-COV-2,
wherein the constant domains CL and CH1 from the antibody or an antigen binding fragment thereof that is capable of binding to the stem helix of a coronavirus, preferably SARS-COV-2, are replaced by each other.

### Clause 30:

A bispecific antibody or an antigen binding fragment thereof according to Clause 29,
wherein the VH domain of the antibody or the antigen binding fragment thereof that is capable of binding to the stem helix of a coronavirus, preferably SARS-COV-2, comprises as CDRs, a Heavy Chain CDR1 region of SEQ ID NO: 055, a Heavy Chain CDR2 region of SEQ ID NO: 137 and a Heavy Chain CDR3 region of SEQ ID NO: 257 and the VL domain of the antibody or the antigen binding fragment thereof that is capable of binding to the stem helix of a coronavirus, preferably SARS-COV-2, comprises as CDRs a Light Chain CDR1 region of SEQ ID NO: 363, a Light Chain CDR2 region of SEQ ID NO: 480 and a Light Chain CDR3 region of SEQ ID NO: 574, and
wherein the VH domain of the antibody or the antigen binding fragment thereof that is capable of binding to the fusion peptide of a coronavirus, preferably SARS-COV-2 comprises as CDRs, a Heavy Chain CDR1 region of SEQ ID NO: 639, a Heavy Chain CDR2 region of SEQ ID NO: 640 and a Heavy Chain CDR3 region of SEQ ID NO: 641 and the VL domain of the antibody or the antigen binding fragment thereof that is capable of binding to the fusion peptide of a coronavirus, preferably SARS-COV-2, comprises as CDRs a Light Chain CDR1 region of SEQ ID NO: 642, a Light Chain CDR2 region of SEQ ID NO: 643 and a Light Chain CDR3 region of SEQ ID NO: 644.

### Clause 31:

A bispecific antibody or an antigen binding fragment thereof according to any of Clauses 26 to 28, comprising:
a) A light chain comprising VL-CL domains and a heavy chain comprising VH-CH1-CH2-CH3 domains of an antibody, capable of binding to the stem helix of a coronavirus, preferably SARS-COV-2; and
b) A light chain comprising VL-CL domains and a heavy chain comprising VH-CH1-CH2-CH3 domains of an antibody, capable of binding to the fusion peptide of a coronavirus, preferably SARS-COV-2, wherein constant domains CL and CH1 from the antibody that is capable of binding to the fusion peptide of a coronavirus, preferably SARS-COV-2 are replaced by each other.

### Clause 32:

A bispecific antibody or an antigen binding fragment thereof according to Clause 31,
wherein the VH domain of the antibody or the antigen binding fragment thereof that is capable of binding to the stem helix of a coronavirus, preferably SARS-COV-2, comprises as CDRs, a Heavy Chain CDR1 region of SEQ ID NO: 055, a Heavy Chain CDR2 region of SEQ ID NO: 137 and a Heavy Chain CDR3 region of SEQ ID NO: 257 and the VL domain of the antibody capable of binding to the stem helix of a coronavirus, preferably SARS-COV-2, comprises as CDRs a Light Chain CDR1 region of SEQ ID NO: 363, a Light Chain CDR2 region of SEQ ID NO: 480 and a Light Chain CDR3 region of SEQ ID NO: 574, and
wherein the VH domain of the antibody capable of binding to the fusion peptide of a coronavirus, preferably SARS-COV-2, comprises as CDRs, a Heavy Chain CDR1 region of SEQ ID NO: 639, a Heavy Chain CDR2 region of SEQ ID NO: 640 and a Heavy Chain CDR3 region of SEQ ID NO: 641 and the VL domain of the antibody capable of binding to the fusion peptide of a coronavirus, preferably SARS-COV-2, comprises as CDRs a Light Chain CDR1 region of SEQ ID NO: 642, a Light Chain CDR2 region of SEQ ID NO: 643 and a Light Chain CDR3 region of SEQ ID NO: 644.

### Clause 33:

A bispecific antibody or an antigen binding fragment thereof according to any of Clauses 26 to 28, comprising:
a) A light chain comprising VL-CL domains and a heavy chain comprising VH-CH1-CH2-CH3 domains of an antibody, capable of binding to the stem helix of a coronavirus, preferably SARS-COV-2; and
b) A light chain comprising VL-CL domains and a heavy chain comprising VH-CH1-CH2-CH3 domains of an antibody, capable of binding to the fusion peptide of a coronavirus, preferably SARS-COV-2, wherein domains VL-CL and VH-CH1 from the antibody that specifically binds to the fusion peptide of a coronavirus, preferably SARS-COV-2, are replaced by each other.

### Clause 34:

A bispecific antibody or an antigen binding fragment thereof according to any of Clauses 26 to 33, wherein the first Fab comprises a heavy chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 629.

### Clause 35:

A bispecific antibody or an antigen binding fragment thereof according to any of Clauses 26 to 33, wherein the second Fab comprises a heavy chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 673.

### Clause 36:

A bispecific antibody or an antigen binding fragment thereof according to any of Clauses 26 to 33, wherein the first Fab comprises a light chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 630.

### Clause 37:

A bispecific antibody or an antigen binding fragment thereof according to any of Clauses 26 to 33, wherein the second Fab comprises a light chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 674.

### Clause 38:

A bispecific antibody or an antigen binding fragment thereof according to any of Clauses 26 to 33, wherein the bispecific antibody or an antigen binding fragment thereof is selected from anyone of the group comprising a full-length antibody, a Fab, modified Fab, Fab′, modified Fab′, F(ab′)2, Fv, single domain antibodies, scFv, scFv-Fc, bi, tri or tetra-valent antibodies, Bis-scFv, diabodies, triabodies, tetrabodies and epitope binding fragments thereof.

### Clause 39:

A method for the production of a bispecific antibody or an antigen binding fragment thereof according to any of Clauses 26 to 38, comprising a) culturing a host cell, comprising an expression vector, comprising a polynucleotide encoding a bispecific antibody or an antibody fragment according to any one of the preceding Clauses under conditions which permit the production of said bispecific antibody or an antigen binding fragment thereof, and b) isolating said bispecific antibody or an antigen binding fragment thereof.

### Clause 40:

A nucleic acid molecule comprising a nucleic acid sequence, wherein the nucleic acid sequence encodes the bispecific antibody or antigen binding fragment thereof, a heavy chain variable region and/or a light chain variable region of the bispecific antibody or an antigen binding fragment thereof according to any of Clauses 26 to 38, preferably wherein the nucleic acid molecule is an isolated nucleic acid molecule.

### Clause 41:

A nucleic acid molecule comprising a nucleic acid sequence, wherein the nucleic acid sequence encodes the bispecific antibody or antigen binding fragment thereof, a heavy chain variable region and/or light chain variable region of the bispecific antibody or an antigen binding fragment thereof according to any of Clauses 26 to 38 and Clause 40, wherein the nucleic acid sequence comprises at least anyone of:
(i) a first nucleic acid sequence encoding the amino acid sequence as set forth in SEQ ID NO. 055, or a first nucleic acid sequence encoding the amino acid sequence having at most 1 or 2 amino acids different from SEQ ID NO. 055,
(ii) a second nucleic acid sequence encoding the amino acid sequence as set forth in SEQ ID NO. 137, or a second nucleic acid sequence encoding the amino acid sequence having at most 1, 2, 3, 4, or 5 amino acids different from SEQ ID NO. 137,
(iii) a third nucleic acid sequence encoding the amino acid sequence as set forth in SEQ ID NO. 257, or a third nucleic acid sequence encoding the amino acid sequence having at most 1, 2 or 3 amino acids different from SEQ ID NO. 257,
(iv) a fourth nucleic acid sequence encoding the amino acid sequence as set forth in SEQ ID NO. 363, or a fourth nucleic acid sequence encoding the amino acid sequence having at most 1, 2, 3 or 4 amino acids different from SEQ ID NO. 363,
(v) a fifth nucleic acid sequence encoding the amino acid sequence as set forth in SEQ ID NO. 480, or a fifth nucleic acid sequence encoding the amino acid sequence having at most 1 or 2 amino acids different from SEQ ID NO. 480,
(vi) a sixth nucleic acid sequence encoding the amino acid sequence as set forth in SEQ ID NO. 574, or a sixth nucleic acid sequence encoding the amino acid sequence having at most 1, 2, 3, or 4 amino acids different from SEQ ID NO. 574,
(vii) a seventh nucleic acid sequence encoding the amino acid sequence as set forth in SEQ ID NO. 639, or a seventh nucleic acid sequence encoding the amino acid sequence having at most 1 or 2 amino acids different from SEQ ID NO. 639,
(viii) an eighth nucleic acid sequence encoding the amino acid sequence as set forth in SEQ ID NO. 640, or an eighth nucleic acid sequence encoding the amino acid sequence having at most 1, 2, 3, 4, or 5 amino acids different from SEQ ID NO. 640,
(ix) a nineth nucleic acid sequence encoding the amino acid sequence as set forth in SEQ ID NO. 641, or a nineth nucleic acid sequence encoding the amino acid sequence having at most 1, 2, 3, or 4 amino acids different from SEQ ID NO. 641,
(x) a tenth nucleic acid sequence encoding the amino acid sequence as set forth in SEQ ID NO. 642, or a tenth nucleic acid sequence encoding the amino acid sequence having at most 1, 2, 3, or 4 amino acids different from SEQ ID NO. 642,
(xi) an eleventh nucleic acid sequence encoding the amino acid sequence as set forth in SEQ ID NO. 643, or an eleventh nucleic acid sequence encoding the amino acid sequence having at most 1 or 2 amino acids different from SEQ ID NO. 643, and
(xii) a twelfth nucleic acid sequence encoding the amino acid sequence as set forth in SEQ ID NO. 644, or a twelfth nucleic acid sequence encoding the amino acid sequence having at most 1, 2, or 3 amino acids different from SEQ ID NO. 644.

### Clause 42:

A bispecific antibody or an antigen binding fragment thereof according to any of Clauses 26 to 38 for use in a method of treating a coronavirus infection in a subject.

### Clause 43:

A composition comprising the bispecific antibody or an antigen binding fragment thereof according to any of Clauses 26 to 38.

### Clause 44:

A composition according to Clause 43 further comprising a second medicament, for simultaneous, separate, or sequential administration.

### Clause 45:

A composition according to Clause 44, wherein the second medicament comprises a second antibody or antigen binding fragment thereof.

### Clause 46:

A composition according to Clause 44, wherein the second medicament comprises a bispecific antibody or bispecific antigen binding fragment thereof.

### Clause 47:

A composition according to Clause 43, wherein the composition further comprises a pharmaceutically acceptable excipient or carrier.

### Clause 48:

A composition according to Clause 43, for use in inhibiting and/or treating a coronavirus infection in a subject, preferably, a SARS-COV-1 or SARS-COV-2 infection in a subject, comprising an effective amount of the bispecific antibody or an antigen binding fragment thereof, nucleic acid molecule, or vector, and optionally a pharmaceutically acceptable excipient or carrier.

### Clause 49:

The composition of Clause 43, for use as a medicament.

### Clause 50:

An inhaler device comprising the composition of Clause 43.

### Clause 51:

A bispecific antibody or an antigen binding fragment thereof according to any of Clauses 26 to 38, wherein the bispecific antibody or an antigen binding fragment thereof is bivalent.

### Clause 52:

A bispecific antibody or an antigen binding fragment thereof according to any of Clauses 26 to 38, wherein the bispecific antibody or antigen-binding fragment thereof is capable of binding to the stem helix and/or the fusion peptide of at least one of an α-coronavirus, ß-coronavirus, γ-coronavirus, and δ-coronavirus, preferably at least an α-coronavirus and/or a ß-coronavirus.

### CLAUSES

The following numbered clauses each represent preferred embodiments of the invention and are part of the description.

### Clause 53:

An anti-coronavirus, preferably anti-SARS-COV-2 bispecific antibody or an antigen binding fragment thereof, having a first Fab capable of binding to the stem helix of a coronavirus, preferably SARS-COV-2 and a second Fab capable of binding to the fusion peptide of a coronavirus, preferably SARS-COV-2, wherein the first Fab comprises a sequence comprising any one or more of preferred SEQ ID Numbers: 055, 137, 257, 363, 480 or 574 and wherein the second Fab comprises a sequence comprising any one or more of preferred SEQ ID Numbers: 645, 646, 647, 648, 649 to 650.

### Clause 54:

A bispecific antibody or an antigen binding fragment thereof according to Clause 53, comprising a first Fab capable of binding to the stem helix of a coronavirus, preferably SARS-COV-2, and a second Fab capable of binding to the fusion peptide of a coronavirus, preferably SARS-COV-2, wherein the first Fab comprises a sequence comprising any one or more of preferred SEQ ID Numbers: 055, 137, 257, 363, 480 and/or 574 and wherein the second Fab comprises a sequence comprising any one or more of preferred SEQ ID Numbers: 645, 646, 647, 648, 649 and/or 650.

### Clause 55:

A bispecific antibody or an antigen binding fragment thereof according to Clauses 53 or 54, comprising a first Fab specifically binding to the stem helix of a coronavirus, preferably SARS-COV-2, and a second Fab specifically binding to the fusion peptide of a coronavirus, preferably SARS-COV-2, characterized in that:
a.) the first Fab comprises a heavy chain variable region comprising as CDRs, a Heavy Chain CDR1 region of SEQ ID NO: 055, a Heavy Chain CDR2 region of SEQ ID NO: 137 and a Heavy Chain CDR3 region of SEQ ID NO: 257 and a light chain variable region comprising as CDRs a Light Chain CDR1 region of SEQ ID NO: 363, a Light Chain CDR2 region of SEQ ID NO: 480 and a Light Chain CDR3 region of SEQ ID NO: 574, and
b.) the second Fab comprises a heavy chain variable region comprising as CDRs, a Heavy Chain CDR1 region of SEQ ID NO: 645, a Heavy Chain CDR2 region of SEQ ID NO: 646 and a Heavy Chain CDR3 region of SEQ ID NO: 647 and a light chain variable region comprising as CDRs a Light Chain CDR1 region of SEQ ID NO: 648, a Light Chain CDR2 region of SEQ ID NO: 649 and a Light Chain CDR3 region of SEQ ID NO: 650.

### Clause 56:

A bispecific antibody or an antigen binding fragment thereof according to any of Clauses 53 to 55, comprising:
a) A light chain comprising VL-CL domains and heavy chain comprising VH-CH1-CH2-CH3 domains of an antibody or an antigen binding fragment thereof, capable of binding to the stem helix of a coronavirus, preferably SARS-COV-2, and
b) A light chain comprising VL-CL domains and heavy chain comprising VH-CH1-CH2-CH3 domains of an antibody or an antigen binding fragment thereof, capable of binding to the fusion peptide of a coronavirus, preferably SARS-COV-2, wherein constant domains CL and CH1 from the antibody or an antigen binding fragment thereof that is capable of binding to the stem helix of a coronavirus, preferably SARS-CoV-2, are replaced by each other.

### Clause 57:

A bispecific antibody or an antigen binding fragment thereof according to Clause 56, wherein the VH domain of the antibody or the antigen binding fragment thereof that is capable of binding to the stem helix of a coronavirus, preferably SARS-COV-2, comprises as CDRs, a Heavy Chain CDR1 region of SEQ ID NO: 055, a Heavy Chain CDR2 region of SEQ ID NO: 137 and a Heavy Chain CDR3 region of SEQ ID NO: 257 and the VL domain of the antibody or the antigen binding fragment thereof that is capable of binding to the stem helix of a coronavirus, preferably SARS-COV-2, comprises as CDRs a Light Chain CDR1 region of SEQ ID NO: 363, a Light Chain CDR2 region of SEQ ID NO: 480 and a Light Chain CDR3 region of SEQ ID NO: 574, and
wherein the VH domain of the antibody or the antigen binding fragment thereof that is capable of binding to the fusion peptide of a coronavirus, preferably SARS-COV-2, comprises as CDRs, a Heavy Chain CDR1 region of SEQ ID NO: 645, a Heavy Chain CDR2 region of SEQ ID NO: 646 and a Heavy Chain CDR3 region of SEQ ID NO: 647 and the VL domain of the antibody or the antigen binding fragment thereof that is capable of binding to the fusion peptide of a coronavirus, preferably SARS-COV-2, comprises as CDRs a Light Chain CDR1 region of SEQ ID NO: 648, a Light Chain CDR2 region of SEQ ID NO: 649 and a Light Chain CDR3 region of SEQ ID NO: 650.

### Clause 58:

A bispecific antibody, or an antigen binding fragment thereof according to any of Clauses 53 to 55, comprising:
a) A light chain comprising VL-CL domains and heavy chain comprising VH-CH1-CH2-CH3 domains of an antibody, capable of binding to the stem helix of a coronavirus, preferably SARS-COV-2; and
b) A light chain comprising VL-CL domains and heavy chain comprising VH-CH1-CH2-CH3 domains of an antibody, capable of binding to the fusion peptide of a coronavirus, preferably SARS-COV-2, wherein constant domains CL and CH1 from the antibody that is capable of binding to the fusion peptide of a coronavirus, preferably SARS-COV-2 are replaced by each other.

### Clause 59:

A bispecific antibody or an antigen binding fragment thereof according to Clause 58, wherein the VH domain of the antibody or the antigen binding fragment thereof that is capable of binding to the stem helix of a coronavirus, preferably SARS-COV-2, comprises as CDRs, a Heavy Chain CDR1 region of SEQ ID NO: 055, a Heavy Chain CDR2 region of SEQ ID NO: 137 and a Heavy Chain CDR3 region of SEQ ID NO: 257 and the VL domain of the antibody capable of binding to the stem helix of a coronavirus, preferably SARS-COV-2, comprises as CDRs a Light Chain CDR1 region of SEQ ID NO: 363, a Light Chain CDR2 region of SEQ ID NO: 480 and a Light Chain CDR3 region of SEQ ID NO: 574, and
wherein the VH domain of the antibody capable of binding to the fusion peptide of a coronavirus, preferably SARS-COV-2, comprises as CDRs, a Heavy Chain CDR1 region of SEQ ID NO: 645, a Heavy Chain CDR2 region of SEQ ID NO: 646 and a Heavy Chain CDR3 region of SEQ ID NO: 647 and the VL domain of the antibody capable of binding to the fusion peptide of a coronavirus, preferably SARS-COV-2, comprises as CDRs a Light Chain CDR1 region of SEQ ID NO: 648, a Light Chain CDR2 region of SEQ ID NO: 649 and a Light Chain CDR3 region of SEQ ID NO: 650.

### Clause 60:

A bispecific antibody or an antigen binding fragment thereof according to any of Clauses 53 to 55, comprising:
a) A light chain comprising VL-CL domains and a heavy chain comprising VH-CH1-CH2-CH3 domains of an antibody, capable of binding to the stem helix of a coronavirus, preferably SARS-COV-2; and
b) A light chain comprising VL-CL domains and a heavy chain comprising VH-CH1-CH2-CH3 domains of an antibody, capable of binding to the fusion peptide of a coronavirus, preferably SARS-COV-2, wherein domains VL-CL and VH-CH1 from the antibody that specifically binds to the fusion peptide of a coronavirus, preferably SARS-COV-2, are replaced by each other.

### Clause 61:

A bispecific antibody or an antigen binding fragment thereof according to any of Clauses 53 to 60, wherein the first Fab comprises a heavy chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 629.

### Clause 62:

A bispecific antibody or an antigen binding fragment thereof according to any of Clauses 53 to 60, wherein the second Fab comprises a heavy chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 675.

### Clause 63:

A bispecific antibody or an antigen binding fragment thereof according to any of Clauses 53 to 60, wherein the first Fab comprises a light chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 630.

### Clause 64:

A bispecific antibody or an antigen binding fragment thereof according to any of Clauses 53 to 60, wherein the second Fab comprises a light chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 676.

### Clause 65:

A bispecific antibody or an antigen binding fragment thereof according to any of Clauses 53 to 60, wherein the bispecific antibody or antigen binding fragment thereof is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

### Clause 66:

A method for the production of a bispecific antibody or an antigen binding fragment thereof according to any of Clauses 53 to 65, comprising a) culturing a host cell, comprising an expression vector, comprising a polynucleotide encoding a bispecific antibody or an antibody fragment according to any one of the preceding Clauses under conditions which permit the production of said bispecific antibody or an antigen binding fragment thereof, and b) isolating said bispecific antibody or an antigen binding fragment thereof.

### Clause 67:

A nucleic acid molecule comprising a nucleic acid sequence, wherein the nucleic acid sequence encodes the bispecific antibody or antigen binding fragment thereof, a heavy chain variable region and/or a light chain variable region of the bispecific antibody or an antigen binding fragment thereof according to any of Clauses 53 to 65, preferably wherein the nucleic acid molecule is an isolated nucleic acid molecule.

### Clause 68:

A nucleic acid molecule comprising a nucleic acid sequence, wherein the nucleic acid sequence encodes the bispecific antibody or antigen binding fragment thereof, a heavy chain variable region and/or light chain variable region of the bispecific antibody or the antigen binding fragment thereof according to any of Clauses 53 to 65 and Clause 67, wherein the nucleic acid sequence comprises at least anyone of:
(i) a first nucleic acid sequence encoding the amino acid sequence as set forth in SEQ ID NO. 055, or a first nucleic acid sequence encoding the amino acid sequence having at most 1 or 2 amino acids different from SEQ ID NO. 055,
(ii) a second nucleic acid sequence encoding the amino acid sequence as set forth in SEQ ID NO. 137, or a second nucleic acid sequence encoding the amino acid sequence having at most 1, 2, 3, 4, or 5 amino acids different from SEQ ID NO. 137,
(iii) a third nucleic acid sequence encoding the amino acid sequence as set forth in SEQ ID NO. 257, or a third nucleic acid sequence encoding the amino acid sequence having at most 1, 2 or 3 amino acids different from SEQ ID NO. 257,
(iv) a fourth nucleic acid sequence encoding the amino acid sequence as set forth in SEQ ID NO. 363, or a fourth nucleic acid sequence encoding the amino acid sequence having at most 1, 2, 3 or 4 amino acids different from SEQ ID NO. 363,
(v) a fifth nucleic acid sequence encoding the amino acid sequence as set forth in SEQ ID NO. 480, or a fifth nucleic acid sequence encoding the amino acid sequence having at most 1 or 2 amino acids different from SEQ ID NO. 480,
(vi) a sixth nucleic acid sequence encoding the amino acid sequence as set forth in SEQ ID NO. 574, or a sixth nucleic acid sequence encoding the amino acid sequence having at most 1, 2, 3, or 4 amino acids different from SEQ ID NO. 574,
(vii) a seventh nucleic acid sequence encoding the amino acid sequence as set forth in SEQ ID NO. 645, or a seventh nucleic acid sequence encoding the amino acid sequence having at most 1 or 2 amino acids different from SEQ ID NO. 645,
(viii) an eighth nucleic acid sequence encoding the amino acid sequence as set forth in SEQ ID NO. 646, or an eighth nucleic acid sequence encoding the amino acid sequence having at most 1, 2, 3, 4, or 5 amino acids different from SEQ ID NO. 646,
(ix) a nineth nucleic acid sequence encoding the amino acid sequence as set forth in SEQ ID NO. 647, or a nineth nucleic acid sequence encoding the amino acid sequence having at most 1, 2, 3, 4, or 5 amino acids different from SEQ ID NO. 647,
(x) a tenth nucleic acid sequence encoding the amino acid sequence as set forth in SEQ ID NO. 648, or a tenth nucleic acid sequence encoding the amino acid sequence having at most 1, 2, or 3 amino acids different from SEQ ID NO. 648,
(xi) an eleventh nucleic acid sequence encoding the amino acid sequence as set forth in SEQ ID NO. 649, or an eleventh nucleic acid sequence encoding the amino acid sequence having at most 1 or 2 amino acids different from SEQ ID NO. 649, and
(xii) a twelfth nucleic acid sequence encoding the amino acid sequence as set forth in SEQ ID NO. 650, or a twelfth nucleic acid sequence encoding the amino acid sequence having at most 1, 2, or 3 amino acids different from SEQ ID NO. 650.

### Clause 69:

A bispecific antibody or an antigen binding fragment thereof according to any of Clauses 53 to 65 for use in a method of treating a coronavirus infection in a subject.

### Clause 70:

A composition comprising the bispecific antibody and/or an antigen binding fragment thereof according to any of Clauses 53 to 65.

### Clause 71:

A composition according to Clause 70 further comprising a second medicament, for simultaneous, separate, or sequential administration.

### Clause 72:

A composition according to Clause 71, wherein the second medicament comprises a second antibody or antigen binding fragment thereof.

### Clause 73:

A composition according to Clause 71, wherein the second medicament comprises a bispecific antibody or bispecific antigen binding fragment thereof.

### Clause 74:

A composition according to Clause 70, wherein the composition further comprises a pharmaceutically acceptable excipient or carrier.

### Clause 75:

A composition according to Clause 70, for use in inhibiting and/or treating a coronavirus infection in a subject, preferably, a SARS-COV-1 or SARS-COV-2 infection in a subject, comprising an effective amount of the bispecific antibody or an antigen binding fragment thereof, nucleic acid molecule, or vector, and optionally a pharmaceutically acceptable excipient or carrier.

### Clause 76:

The composition of Clause 70, for use as a medicament.

### Clause 77:

An inhaler device comprising the composition of Clause 70.

### Clause 78:

A bispecific antibody or an antigen binding fragment thereof according to any of Clauses 53 to 65, wherein the bispecific antibody or an antigen binding fragment thereof is bivalent.

### Clause 79:

A bispecific antibody or an antigen binding fragment thereof according to any of Clauses 53 to 65, wherein the bispecific antibody or antigen binding fragment thereof is capable of binding to the stem helix and/or the fusion peptide of at least one of an α-coronavirus, ß-coronavirus, γ-coronavirus, and δ-coronavirus, preferably at least an α-coronavirus and/or a ß-coronavirus.

### DESCRIPTION OF THE DRAWINGS

FIG. 1. Survival after lethal challenge-CV3-25.

Kaplan-Meier survival curves of the intraperitoneal prophylactic treatment group. Animals (n=10 per group), treated with a dose titration with the control antibody, CV3-25, at day −1. At day 0, animals were infected with 10^{3.5} TCID₅₀ of SARS-COV-2 Delta. A vehicle control group was included (PBS). Lines are nudged relative to the Y-axis to improve visual representation.

FIG. 2. Survival after lethal challenge-CV3-25.

Kaplan-Meier survival curves of the intranasal prophylactic treatment group. Animals (n=10 per group, except 15 mg/kg where n=8), treated with a dose titration with the control antibody, CV3-25, at day −1. At day 0, animals were infected with 10^{3.5} TCID₅₀ of SARS-COV-2 Delta. A vehicle control group was included (PBS). Lines are nudged relative to the Y-axis to improve visual representation.

FIG. 3. Survival after lethal challenge-Antibody according to the invention.

Kaplan-Meier survival curves of the intraperitoneal prophylactic treatment group. Animals (n=10 per group), treated with a dose titration with the antibody having heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574, at day −1. At day 0, animals were infected with 10^{3.5} TCID₅₀ of SARS-COV-2 Delta. A vehicle control group was included (PBS). Lines are nudged relative to the Y-axis to improve visual representation.

FIG. 4. Survival after lethal challenge-Antibody according to the invention.

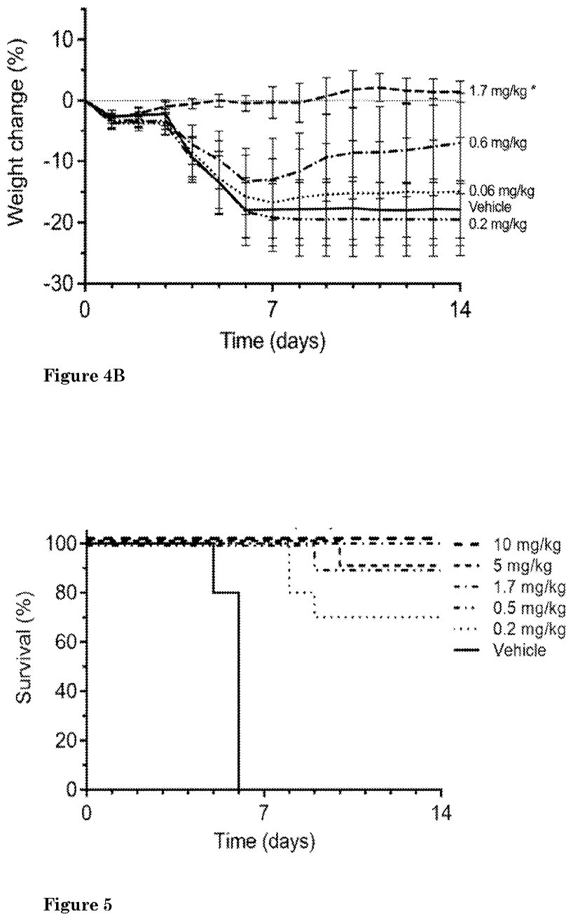

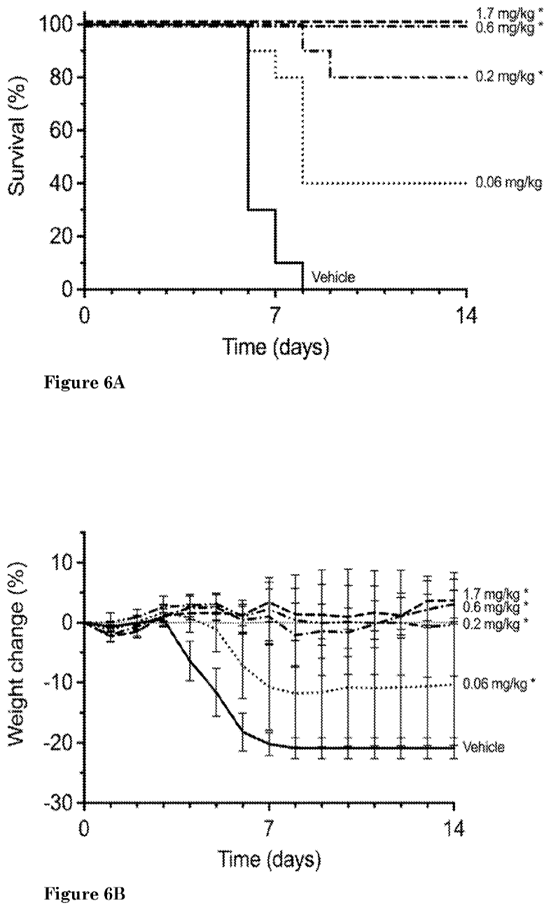

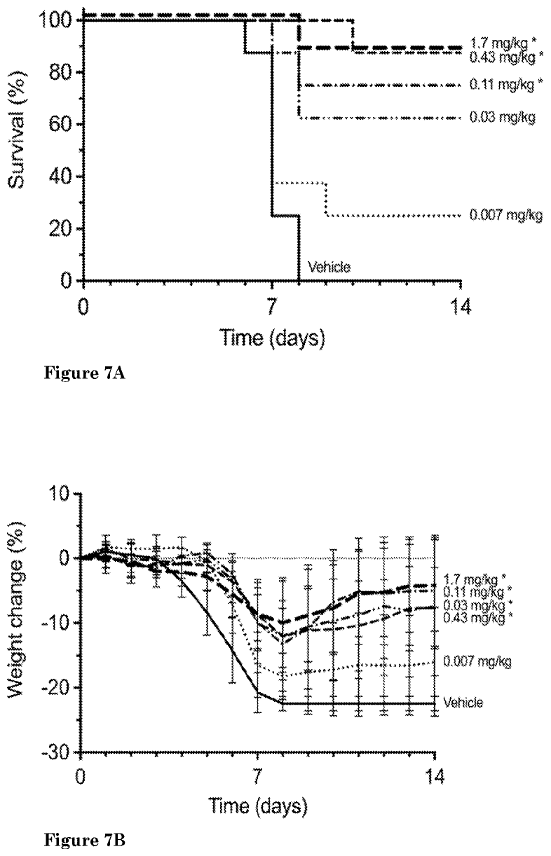

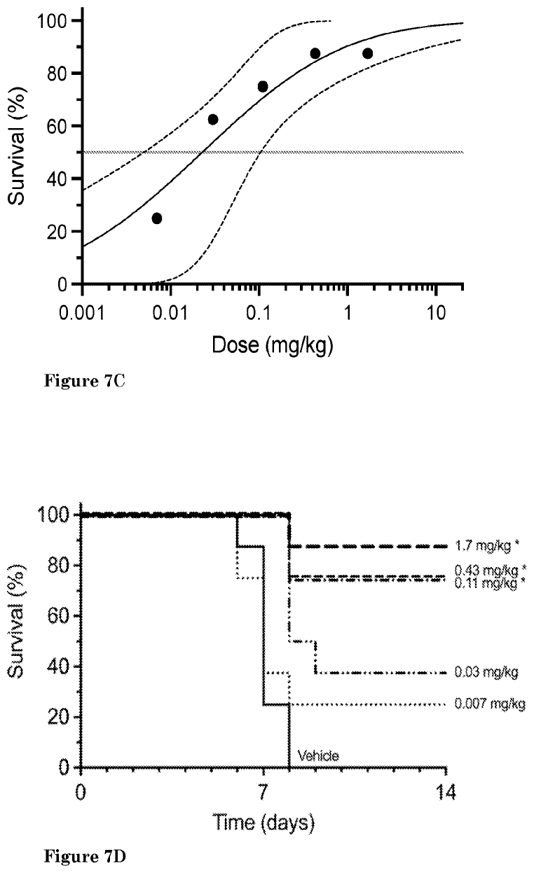

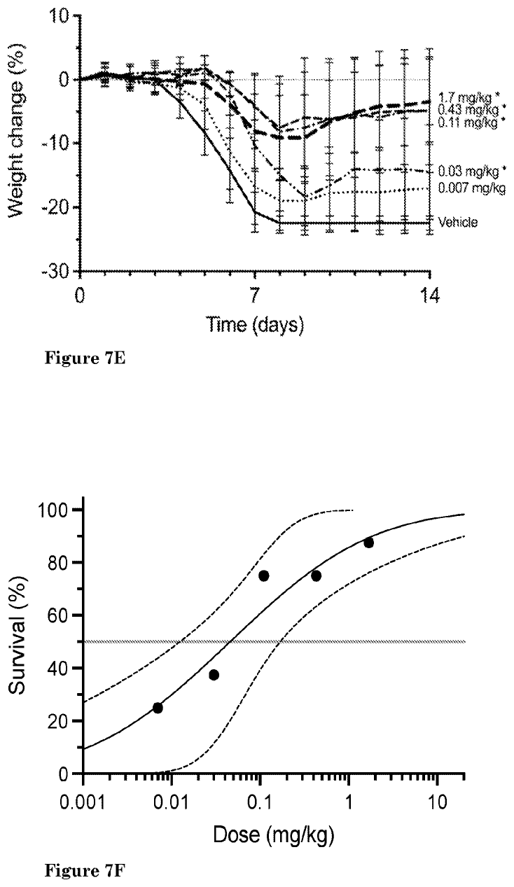

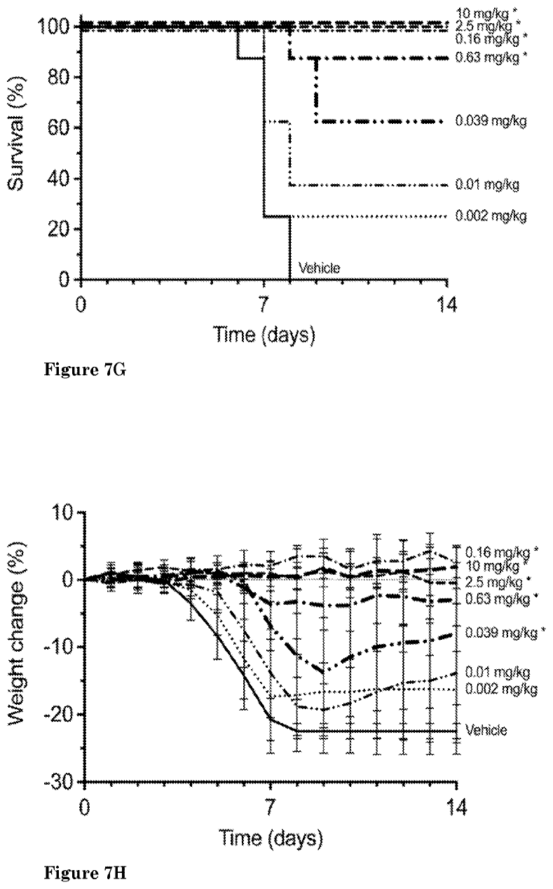

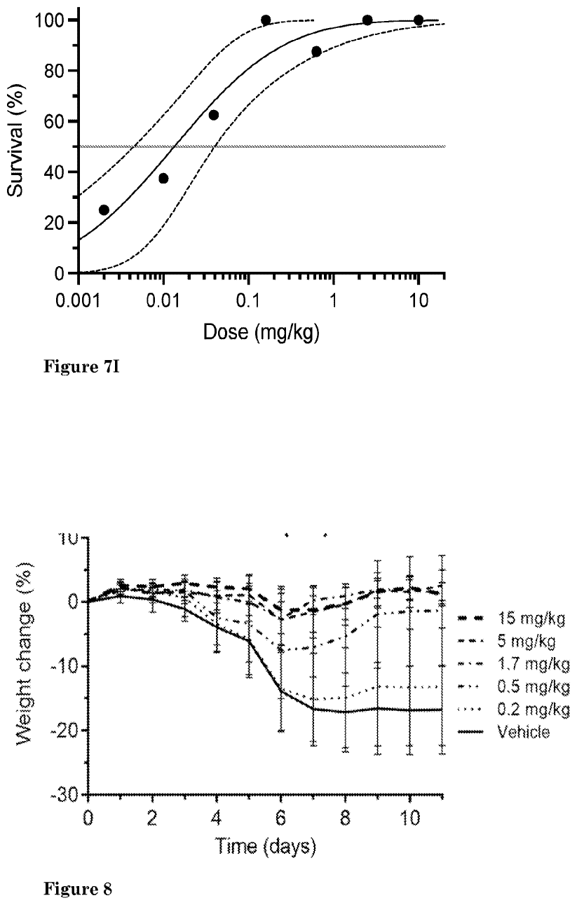

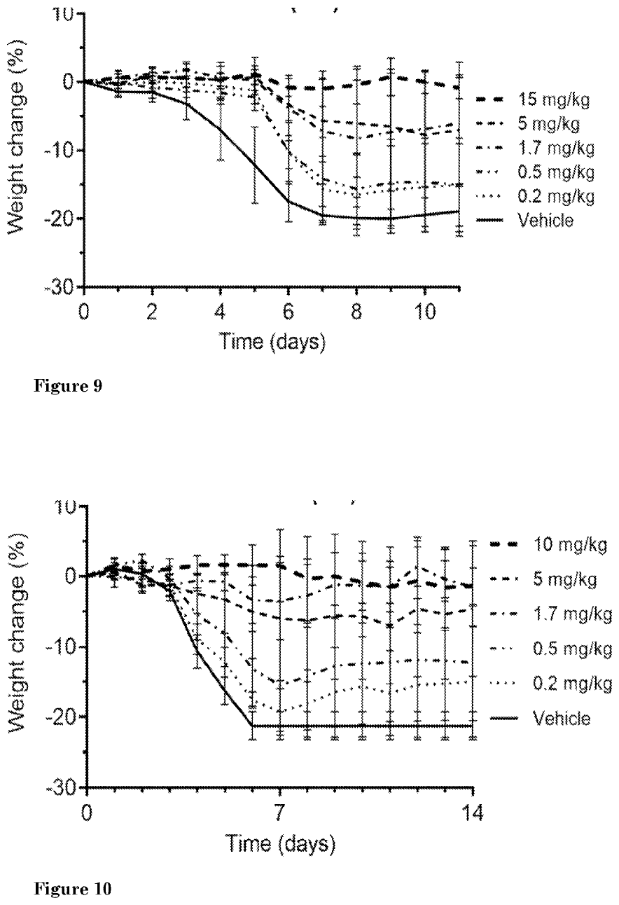

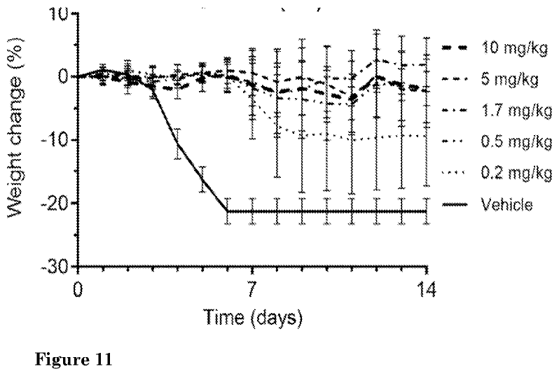

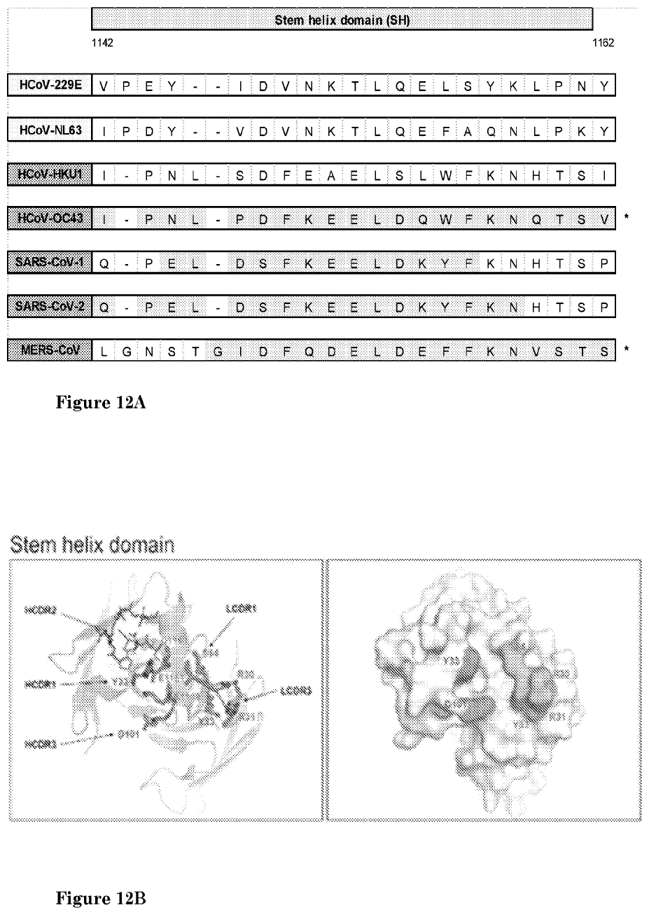

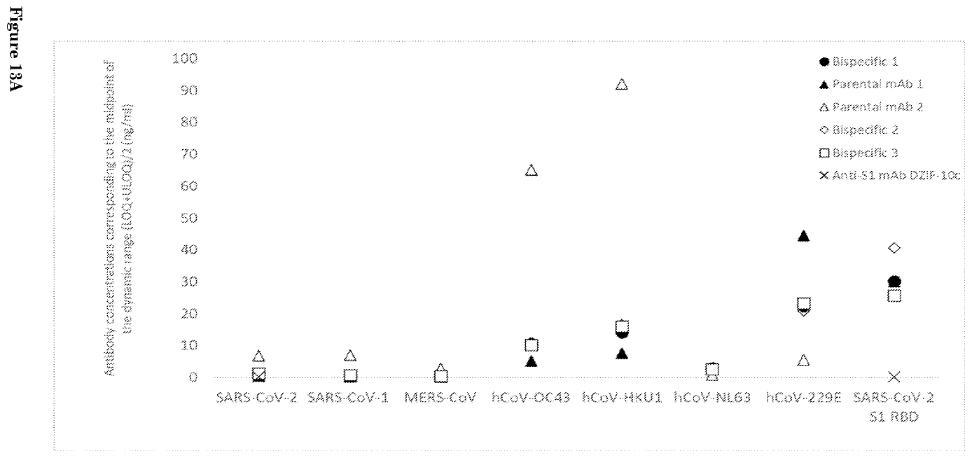

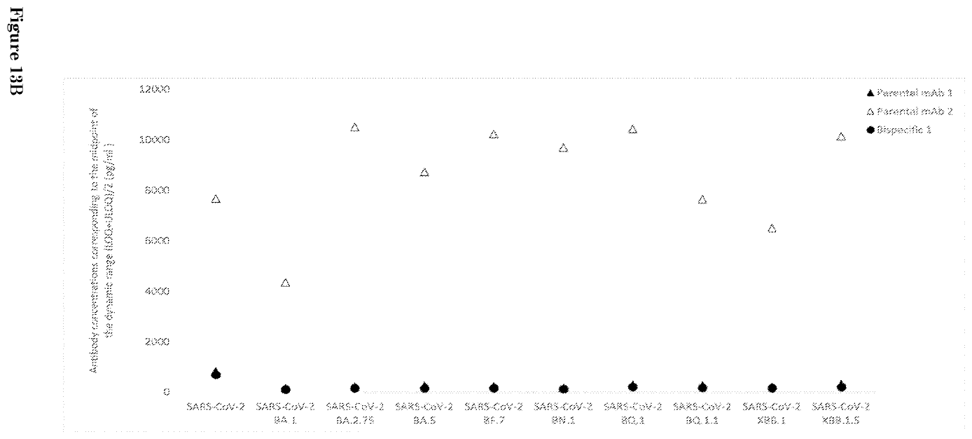

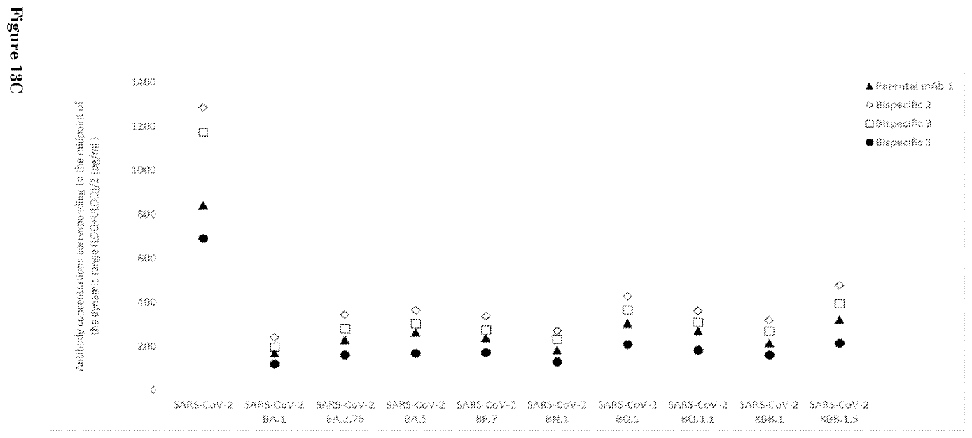

Survival and bodyweight change for the intraperitoneal administration of the antibody according to invention with the following CDRs as described in SEQ ID Numbers: 055, 137, 257, 363, 480 and 574 in a pre-exposure efficacy study in K18-hACE2 mice upon lethal SARS-COV-2 Delta challenge. Animals (n=10 per treatment group) were treated intraperitoneally with a dose titration of the antibody according to invention at day −1. At day 0, animals were infected with 10^{3.5} TCID₅₀ of SARS COV-2 Delta. A vehicle control group (n=10) was included (PBS). FIG. 4A: Kaplan-Meier survival curves of the treatment groups is shown. Lines are nudged relative to the Y-axis to improve visual representation. FIG. 4B: Bodyweight change (%) relative to day 0 is shown. Error bars represent the mean with 95% confidence interval. If a mouse died/was euthanized during follow-up of the study, the last observed bodyweight was carried forward. Asterisk indicates significance compared to control group.

FIG. 5. Survival after lethal challenge-Antibody according to the invention.

Kaplan-Meier survival curves of the intranasal prophylactic treatment group. Animals (n=10 per group), treated with a dose titration with the antibody having heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574, at day −1. At day 0, animals were infected with 10^{3.5} TCID₅₀ of SARS-COV-2 Delta. A vehicle control group was included (PBS). Lines are nudged relative to the Y-axis to improve visual representation.

FIG. 6. Survival and bodyweight change for the intranasal administration of the antibody according to invention

Survival and bodyweight change for the intranasal administration of the antibody according to invention with the following CDRs as described in SEQ ID Numbers: 055, 137, 257, 363, 480 and 574 in a pre-exposure efficacy study in K18-hACE2 mice upon lethal SARS-COV-2 Delta challenge. Animals (n=10 per treatment group, except for n=9 for doses of 0.6 mg/kg) were treated intranasally with a dose titration of the antibody according to invention at day −1. At day 0, animals were infected with 10^{3.5} TCID₅₀ of SARS CoV-2 Delta. A vehicle control group (n=10) was included (PBS). FIG. 6A: Kaplan-Meier survival curves of the treatment groups is shown. Lines are nudged relative to the Y-axis to improve visual representation. FIG. 6B: Bodyweight change (%) relative to day 0 is shown. Error bars represent the mean with 95% confidence interval. If a mouse died/was euthanized during follow-up of the study, the last observed bodyweight was carried forward.

FIG. 7. Survival and bodyweight change for the intranasal administration of two parental antibodies and a bispecific antibody according to the invention

Survival and bodyweight change for the intranasal administration of two parental antibodies and a bispecific antibody according to invention in a pre-exposure efficacy study in K18-hACE2 mice upon lethal SARS-COV-2 Delta challenge. Animals (n=8 per group) were treated with a dose titration of either the Parental mAb 1 (FIG. 7A-7C), Parental mAb 2 (FIGS. 7D-7F) or bispecific antibody (FIGS. 7G-7I) at day −1 intranasally. At day 0 animals were infected with 10^{3.5} TCID₅₀ of SARS COV-2 Delta. A vehicle control group (n=10) was included (PBS). FIGS. 7A, 7D, and 7G: Kaplan-Meier survival curves of the treatment groups is shown. Lines are nudged relative to the Y-axis to improve visual representation. FIGS. 7B, 7E, and 7H: Bodyweight change (%) relative to day 0 is shown. Error bars represent the mean with 95% confidence interval. If a mouse died/was euthanized during follow-up of the study, the last observed bodyweight was carried forward. FIGS. 7G, 7H, and 7I: The survival dose response curves using Probit regression without assuming a parallel dose response curve between treatments is shown. The vehicle control group was included in the modelling at a dose of 0 mg/kg. The 95% pointwise fiducial confidence intervals (CI) for the predicted dose are indicated. Asterisk indicates significance compared to the control group.

FIG. 8. Bodyweight change-CV3-25.

Bodyweight change (%) relative to day 0, for the intraperitoneal prophylactic treatment group. Animals (n=10 per group) were treated with a dose titration of the control antibody, CV3-25, at day −1 intranasally. At day 0 animals were infected with 10^{3.5} TCID₅₀ of SARS-CoV-2 Delta. A vehicle control group was included (PBS). Bars represent the 95% CI. of the mean. If a mouse died/was euthanized during follow-up of the study, the last observed bodyweight was carried forward.

FIG. 9. Bodyweight change-CV3-25.

Bodyweight change (%) relative to day 0, for the intranasal prophylactic treatment group. Animals (n=10 per group, except 15 mg/kg where n=8) were treated with a dose titration of the control antibody, CV3-25, at day −1 intranasally. At day 0 animals were infected with 10^{3.5} TCID₅₀ of SARS-COV-2 Delta. A vehicle control group was included (PBS). Bars represent the 95% CI. of the mean. If a mouse died/was euthanized during follow-up of the study, the last observed bodyweight was carried forward.

FIG. 10. Bodyweight change-Antibody according to the invention.

Bodyweight change (%) relative to day 0, for the intraperitoneal prophylactic treatment group. Animals (n=10 per group) were treated with a dose titration of the antibody having heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574, at day −1 intranasally. At day 0 animals were infected with 10^{3.5} TCID₅₀ of SARS-COV-2 Delta. A vehicle control group was included (PBS). Bars represent the 95% CI. of the mean. If a mouse died/was euthanized during follow-up of the study, the last observed bodyweight was carried forward.

FIG. 11. Bodyweight change-Antibody according to the invention.

Bodyweight change (%) relative to day 0, for the intranasal prophylactic treatment group. Animals (n=10 per group) were treated with a dose titration of the antibody having heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574, at day −1 intranasally. At day 0 animals were infected with 10^{3.5} TCID₅₀ of SARS-COV-2 Delta. A vehicle control group was included (PBS). Bars represent the 95% CI. of the mean. If a mouse died/was euthanized during follow-up of the study, the last observed bodyweight was carried forward.

FIG. 12: Linear epitope mapping against Spike helix domain of alpha and beta coronaviruses.

Grey highlighted amino acids were identified as the epitope where the antibody was binding the Spike protein. Target sequences for the 7 tested strains were aligned by Clustal. Asterisk indicates that this sequence may contain multiple smaller parts of the epitope. Epitope footprints (FIG. 12A) and paratope hotspots (FIG. 12B) for the antibody against the SH domain of SARS-COV-2. CDRs for heavy and light chains are indicated.

FIG. 13: Graphical representation of the data presented in Example 17. FIG. 13A is a graphical representation of the extent to which the Parental mAb 1 (▴) and Parental mAb 2 (Δ), bispecific antibody Bispecific 1 (●) according to the invention, bispecific antibody Bispecific 2 (⋄) according to the invention, bispecific antibody Bispecific 3 (□) according to the invention, and the anti-S1 mAb DZIF-10c (x) bind to a selection of alpha coronaviruses and beta coronaviruses. FIG. 13B is a graphical representation of the extent to which the Parental mAb 1 (▴) and Parental mAb 2 (Δ), and bispecific antibody Bispecific 1 (●) bind to a selection of omicron variants. FIG. 13C is a graphical representation of the extent to which the Parental mAb 1 (▴), bispecific antibody Bispecific 1 (●) according to the invention, bispecific antibody Bispecific 2 (⋄) according to the invention, and bispecific antibody Bispecific 3 (□) according to the invention, bind to a selection of bind to a selection of omicron variants.

### DETAILED DESCRIPTION OF THE INVENTION

For the purpose of clarity and a concise description, features may be described herein as part of the same or separate embodiments, however, it will be appreciated that the disclosure includes embodiments having combinations of all or some of the features described. References to “method for treatment”, “treatment method”, “antibody for use” and “use of an antibody in the manufacture of a medicament” can be used interchangeably and embodiments disclosed in relation to any one of those aspects also applies in relation to said other aspects. In other words, they all refer to medical treatments involving an antibody as disclosed herein.

### Prophylactic Treatment

The term ‘prophylactic treatment’, as used herein, includes reference to a treatment for preventing infection of an individual with a coronavirus, or preventing symptoms after infection with a coronavirus, or preventing severe symptoms after known infection with a coronavirus with or without symptoms, or preventing hospitalization and death after infection with a coronavirus.

Prevention of an infection is preferably performed by administration of an antibody as disclosed herein prior to coronavirus exposure i.e. pre-exposure prophylaxis.

‘Prophylactically’ therefore preferably means prior to virus exposure. Nonetheless, it may also involve administration after infection, for example for reducing the replication or spread, or increasing clearance of the virus i.e. post-exposure prophylaxis.

Infected individuals may present with no symptoms. Alternatively, infected individuals may present with symptoms.

Preferably, post-exposure prophylaxis involves administering an antibody as disclosed herein after coronavirus exposure to prevent symptomatic disease.

Preferably, post-exposure prophylaxis involves administering an antibody as disclosed herein after coronavirus exposure to prevent severe disease, in particular hospitalization.

In a treatment method of the invention, prophylactic treatment involves administration of antibodies against coronavirus at a point in time when the individual is not infected with coronavirus. Preferably, said antibodies bind to a conserved epitope of the spike protein of a coronavirus virion. In some embodiments, an individual in need thereof is not (yet) infected with coronavirus.

### Therapeutic Treatment

The term ‘therapeutic treatment’, as used herein, includes reference to treatment of a viral infection (including coronavirus disease) after viral infection has taken place. A viral infection involves the entry of the body by the virus, and/or the replication of the virus in the body and/or the spreading of the virus to cells, tissues or locations in the body that were previously uninfected. A viral infection may cause one or more disease, but may also be latent, in other words may reside in the body without causing symptoms or disease.

### Coronavirus

The term ‘coronavirus’, as used herein, includes reference to a positive-sense single-stranded RNA virus belonging to the family of Coronaviridae.

Preferably, an antibody as disclosed herein is capable of specifically binding to the stem helix of a coronavirus, in particular SARS-COV-2.

Preferably, an antibody as disclosed herein is capable of specifically binding to the stem helix of SARS-COV-2 variants of concern.

Preferably, the antibody as disclosed herein is capable of neutralizing a coronavirus, in particular SARS-COV-2.

Preferably, an antibody as disclosed herein is capable of neutralizing at least one or more, preferably two or more, preferably three or more, preferably four or more, even more preferably five or more coronavirus subtypes.

The phrase “is capable of” as used in, means the ability of an antibody or an antigen-binding fragment thereof as described herein is enough to bind to at least a part of an antigen, such as the stem helix of a coronavirus, in particular SARS-COV-2 and/or the fusion protein of a coronavirus, in particular SARS-COV-2. The binding affinity or the binding ability of the antibody and/or the antigen-binding fragment thereof as described herein can be measured using any methods known by a person skilled in the art. The phrase “is capable of” as used here, encompasses but not limited to the phrase “specifically binding” or “specifically bind to”.

### Coronavirus Infection

The term ‘coronavirus infection’, as used herein, includes reference to the pathological or non-pathological, preferably pathological, entrance and residence of a coronavirus of any type in a human host. The infecting virus may replicate within the host, its cells or the cells of its microbiome. The infecting virus may or may not cause a disease, for example COVID-19. The infection may or may not be able to be detected by methods for virus infection detection known in the art. The infected individual may or may not be aware of the infection. Typical, but non-exclusive locations of the human body where, for example SARS-COV-2, may be located in an infected individual, are the respiratory system and/or cells thereof and the cardiovascular system and/or cells thereof. The term ‘coronavirus infection’, as used herein, further includes reference to the entrance and residence of a part of a coronavirus of any type that is able to cause viral replication in a human host. The term ‘coronavirus infection’ encompasses symptoms or disease following the infection, e.g. COVID-19. The term ‘coronavirus infection’ encompasses ‘SARS-COV-2 infection’.

### Epitope

An “epitope”, as used herein, includes reference to a moiety that is capable of binding to an antibody as disclosed herein with sufficiently high affinity to form a detectable antigen-antibody complex.

### Individual

The term ‘individual’, as used herein, includes reference to a mammal or human that is subject to, or a risk of suffering from viral infection. Infection may take place in any system, tissue or cell belonging to the host, including the host's microbiome. Coronavirus infection and the disease coronavirus may occur in individuals of all age groups and sexes. Nonetheless, preferably, the individual is a human, in particular an elderly human such as a human that is at least 60, 65, 70, 75, 80, or at least 85 years old, or that has an increased risk of infection because of occupation or living environment. Preferably, the individual is at risk of suffering from severe illness, for example COVID-19, once infected. In preferred embodiments, the individual has an underlying disease such as (i) a respiratory disease such as asthma, COPD, chronic bronchitis and lung emphysema, (ii) cardiovascular disease such as cardiac arrhythmia or individuals that have received cardiac surgery, (iii) diabetes, (iv) renal failure and/or (v) a disease affecting the immune system, for instance immunocompromised individuals, or higher risk of viral infection because of occupation.

The phrase ‘an individual’, as used herein, includes reference to a mammal such as a but not limited to a human that benefits from a specified therapy, for example, the phrase ‘an individual’ may encompass Non-human primates (NHP).

Preferably, the individual is a mammal, more preferably a human.

### Administering and Administration

The terms ‘administering’ and ‘administration’, as used herein, include reference to the provision of one or more drug and optionally one or more adjuvant with the aim to treat, cure, reduce, or prevent a disease or its symptoms in an individual, or to promote the individual's well-being. Preferred methods of administration of the antibody as disclosed herein include mucosal administration, preferably intranasal administration and oral inhalation.

### An Individual in Need Thereof

The phrase ‘an individual in need thereof’, as used herein, includes reference to a mammal such as a human that benefits from a specified therapy. A treatment method of the invention may be used prophylactically, the exhibition of symptoms or indications for coronavirus infection are not required. Individuals that are especially in need of the method or antibody for use of the invention, are individuals with an elevated risk of coronavirus infection, individuals with an elevated risk of developing severe symptoms (illness), for example COVID-19, and/or individuals with an elevated risk of dying from COVID-19. The person skilled in the art is aware of the risk factors for an elevated risk of coronavirus infection, an elevated risk of developing severe symptoms of coronavirus infection, and an elevated risk of dying from coronavirus infection.

### Antibody

The term ‘antibody’, as used herein, includes reference to an intact immunoglobulin, including monoclonal antibodies, such as chimeric, humanized or human monoclonal antibodies, or to a binding molecule comprising an antigen-binding domain (such as heavy chain CDRs 1-3 of a variable domain) of an antibody as disclosed herein or an antibody that competes with an antibody as disclosed herein for specific binding to the binding partner of the immunoglobulin.

In other words, functional fragments of antibodies are also encompassed by the term ‘antibody’. Antibodies are generally Y-shaped proteins. Within the antibody, constant domain and variable domains are generally present. The variable domain facilitates antigen binding. An antibody generally comprises two heavy chains and two light chains. Both the heavy chains and the light chains are partially constant and partially variable. Antibodies occur in a few classes: IgA, IgD, IgE, IgG and IgM. Preferably, the antibody of the invention is of the IgG, preferably IgG1, class. Some classes may be further subdivided into subclasses or isotypes. For example, the IgG class is subdivided into the subclasses IgG1, IgG2, IgG3 and IgG4. Preferably, the antibody of the invention is of the IgG, preferably IgG1, class. Antigen-binding regions, or antigen-binding fragments of an antibody, which are encompassed by the term ‘antibody’ and which are therefore part of the present invention, may include, for example, Fab, F(ab′), F(ab′)2, dAb, Fv, Fd, CDR fragments, diabodies, triabodies, tetrabodies, single-chain antibodies (scFv), scFv-Fc, bivalent single-chain antibodies, single-chain phage antibodies, (poly) peptides that contain at least a fragment of an immunoglobulin that is sufficient to confer specific antigen binding to the (poly) peptide, etc. The above fragments may be produced synthetically or by enzymatic or chemical cleavage of intact immunoglobulins or they may be genetically engineered by recombinant DNA techniques. The methods for production of antibodies and antigen-binding fragments are well-known to a person skilled in the art. The antibody may be conjugated or unconjugated. The antibody may be conjugated, linked, or otherwise physically or functionally associated with an effector moiety or tag, such as inter alia an enzyme, a liposome, a radioactive substance, a fluorophore, a toxic substance. Antibodies may have been stabilized, multimerized, humanized or otherwise manipulated.

Antibodies may be neutralizing, which includes reference to inhibition of a virus as measured by an in vitro neutralization assay, for instance in terms of viral host cell entry and/or viral replication. Neutralization can for example be achieved by inhibiting the attachment or adhesion of the virus to the cell surface, or by inhibition of the fusion of viral and cellular membranes following attachment of the virus to the target cell or by inhibiting viral egress from cells. Neutralization does not specify the method of neutralization. Preferably, the antibody is cross-neutralizing, which includes reference to the ability of the antibodies of the invention to bind and neutralize a set of different molecules, preferably different molecules of different subtypes belonging to the Coronaviridae family.

The term “domain” or “region” as used herein, with reference to a part of an antibody, bispecific antibody, or an antigen binding fragment thereof, can be used interchangeably herein.

The phrase “antigen-binding fragments”, “antigen binding fragments”, “antigen fragment”, “a fragment of an antibody” or “antigen-binding molecule” as used herein, means a protein, polypeptide or molecular complex comprising or consisting of at least one complementarity determining region (CDR) that alone, or in combination with one or more additional CDRs and/or framework regions (FRs), as described herein, is capable of binding to a part of an antigen, including the stem helix of a coronavirus, in particular SARS-COV-2, and/or the stem helix of a coronavirus, in particular SARS-COV-2 and its variants of concern. The phrases “antigen-binding fragments”, “antigen binding fragments”, “antigen fragment”, “a fragment of an antibody” or “antigen-binding molecule” as used herein, can be used interchangeably. For instance, the phrase “antigen binding fragments” as used herein is to be used interchangeably with the phrase “antibody fragments” as used herein.

Preferably, the antibody of the invention as disclosed herein can cross-neutralize coronavirus, in particular SARS-COV-2.

Antibodies comprise complementarity determining regions situated on the variable domains of the heavy chain and the light chain. The CDRs contribute to a large extent to the antigen binding site. Three CDRs can be distinguished, namely CDR1, CDR2 and CDR3. As each CDR can be located on either the light chain or the heavy chain, there are generally six CDRs for each antigen receptor that collectively contact the antigen: the light chain CDR1, the light chain CDR2, the light chain CDR3, the heavy chain CDR1, the heavy chain CDR2 and the heavy chain CDR3. The CDRs of type CDR3 are the most variable. The CDRs can be specific for linear epitopes, discontinuous epitopes, or conformational epitopes of proteins or protein fragments, either as present on the protein in its native conformation or, in some cases, as present on the proteins as denatured or activated. Epitopes may also consist of or comprise post-translational modifications of proteins. Antibodies of the invention that are of particular interest, are antibodies comprising CDRs that recognize coronavirus antigens, such as the spike protein and in particular the stem helix of the spike protein. The antibody as disclosed herein binds to a conserved epitope in the stem helix at the base of the spike protein of the SARS-COV-2 virion. The epitope of the antibody as disclosed herein uses light and heavy chain CDR loops.

The antibody as disclosed herein can be used in isolated or non-isolated form.

Preferably, the compositions of the invention comprise a single anti-coronavirus antibody as disclosed herein.

Furthermore, the antibody as disclosed herein can be used alone or in a mixture comprising the antibody (or variant, fragment or bispecific thereof) as disclosed herein, and/or with other antibodies that bind to a coronavirus and have a coronavirus inhibiting effect. In other words, the antibody as disclosed herein can be used in combination, e.g., as a pharmaceutical composition or co-administration of compositions comprising two or more antibodies that specifically bind coronavirus. For example, antibodies having different, but complementary activities can be combined in a single therapy to achieve a desired therapeutic or prophylactic effect, but alternatively, antibodies having identical activities can also be combined in a single therapy to achieve a desired prophylactic or therapeutic effect. Optionally, the mixture further comprises at least one other therapeutic agent.

Preferably, the antibody as disclosed herein are a bispecific antibody and/or an antigen binding fragment thereof, wherein the bispecific antibody is capable of binding to the stem helix of a coronavirus and/or capable of binding to fusion peptide of coronavirus. The phrase “capable of binding” encompasses the phase “specifically binds to”, in other words, an antibody, a bispecific antibody and/or an antigen binding fragment thereof as disclosed herein are that is capable of binding to an epitope may also be capable of binding the other epitopes.

Preferably, the coronavirus as described herein includes SARS-COV-2 and variants.

The phrase “are replaced by each other”, “are mutually interchanged”, “swap places with each other”, or “are traded for one another” as used herein, includes reference to at least a part of the heavy chain domains of an antibody or an antigen-binding fragment as described herein such as when a CH1 and/or VH, is substituted by at least a part of the light chain domains such as CL and/or VL to which said at least a part of the heavy chain domains are paired, and at the same instant, said at least a part of the light chain domains is substituted by said at least a part of the heavy chain domains. The phrase “are replaced by each other”, “are mutually interchanged”, “swap places with each other”, or “are traded for one another” are to be used interchangeably herein.

Preferably, the antibody as disclosed herein is a human antibody.

### Framework Regions

Antibodies also comprise framework regions, generally four framework regions (FR1, FR2, FR3 and FR4) on each of the variable heavy chain domain and variable light chain domain. The CDRs are situated between the framework regions (preferably in the order FR1-CDR1-FR2-CDR2-FR3-CDR3-FR4) and both the CDRs and framework regions together define a heavy chain variable domain (or region) and light chain variable domain (or region) which may also be referred to as an antigen-binding region or domain.

### IgG Antibody

The term ‘IgG antibody’, as used herein, includes reference to an antibody comprising two antigen-binding sites. IgG is the most common antibody in human serum. Heavy chains of IgG antibodies are of type γ, which can be subdivided in γ1, γ2, γ3 and γ4. Preferably, the antibodies described herein are γ1 heavy chains. Light chains of IgG antibodies are of type λ or κ. Preferably, the antibodies described herein are A light chains.

Preferably, the antibody is an IgG antibody, preferably an IgG1 antibody.

### IgA Antibody

The term ‘IgA antibody’, as used herein, includes reference to an antibody comprising two to four antigen-binding sites. IgA is the most abundant antibody in mucosal secretions. An IgA antibody can be produced in monomeric, dimeric or secretory form, with two or four antigen-binding sites, respectively, and can exist as an IgA1 or IgA2 isotype. [Heavy chains of IgA antibodies are of type α, which can be subdivided in α1 and α2. Light chains of IgA antibodies are of type λ or κ.

Preferably, the antibody is an IgA antibody.

### IgM Antibody

The term ‘IgM antibody’, as used herein, includes reference to an antibody comprising ten to twelve antigen-binding sites. An IgM antibody can be produced in pentameric or hexameric form. Heavy chains of IgM antibodies are of type μ. Light chains of IgM antibodies are of type λ or κ.

Preferably, the antibody is an IgM antibody.

### Anti-Coronavirus Antibody

The term ‘anti-coronavirus antibody’, as used herein, includes reference to an antibody as disclosed herein, i.e. an antibody that can be used in a treatment method of the invention. Preferably, the anti-coronavirus antibody of the invention is able to treat different subtypes of coronavirus.

For the avoidance of doubt, the term ‘anti-coronavirus antibody’, can be used interchangeably with ‘coronavirus antibody’.

### A Composition

In a preferred embodiment, an antibody disclosed herein is the sole active ingredient in a composition administered for treatment, e.g., the antibody is provided in a composition as the sole active ingredient.

In a preferred embodiment, the active ingredients comprise an antibody disclosed herein, e.g., the antibody is provided either as a composition as the sole active ingredient in a composition administered for treatment or in a combination with another antibody.

In a preferred embodiment, a first antibody as disclosed herein is provided as a composition in combination with a second antibody, wherein the first antibody comprises a CDR sequence comprising any one of SEQ ID NO: 001 to 006, 012 to 017, 023 to 028, 033 to 038, 042 to 047, 050 to 055, 077 to 082, 088 to 093, 099 to 104, 110 to 115, 121 to 126, 132 to 137, 197 to 202, 208 to 213, 219 to 224, 230 to 235, 241 to 246, 252 to 257, 303 to 308, 314 to 319, 325 to 330, 336 to 341, 347 to 352, 358 to 363, 421 to 426, 432 to 437, 443 to 448, 454 to 459, 465 to 470, 475 to 480, 514 to 519, 525 to 530, 536 to 541, 547 to 552, 558 to 563, 569 to 574. More preferably wherein the first antibody comprises CDR sequences comprising any one or more of SEQ ID NO: 055, 137, 257, 363, 480 or 574, and the second antibody comprises CDR sequences comprising any one or more of SEQ ID NO: 639, 640, 641, 642, 643 or SEQ ID NO.: 644. Preferably wherein the composition is administered for prophylactic and/or therapeutic treatment.

In a preferred embodiment, a first antibody as disclosed herein is provided as a composition in combination with a second antibody, wherein the first antibody comprises a CDR sequence comprising any one of SEQ ID NO: 001 to 006, 012 to 017, 023 to 028, 033 to 038, 042 to 047, 050 to 055, 077 to 082, 088 to 093, 099 to 104, 110 to 115, 121 to 126, 132 to 137, 197 to 202, 208 to 213, 219 to 224, 230 to 235, 241 to 246, 252 to 257, 303 to 308, 314 to 319, 325 to 330, 336 to 341, 347 to 352, 358 to 363, 421 to 426, 432 to 437, 443 to 448, 454 to 459, 465 to 470, 475 to 480, 514 to 519, 525 to 530, 536 to 541, 547 to 552, 558 to 563, 569 to 574. More preferably wherein the first antibody comprises CDR sequences comprising any one or more of SEQ ID NO: 055, 137, 257, 363, 480 or 574, and the second antibody comprises CDR sequences comprising any one or more of SEQ ID NO: 645, 646, 647, 648, 649 or SEQ ID NO.: 650. Preferably wherein the composition is administered for prophylactic and/or therapeutic treatment.

In a preferred embodiment, a first antibody as disclosed herein is provided as a composition in combination with a second antibody, wherein the first antibody comprises a CDR sequence comprising any one of SEQ ID NO: 001 to 006, 012 to 017, 023 to 028, 033 to 038, 042 to 047, 050 to 055, 077 to 082, 088 to 093, 099 to 104, 110 to 115, 121 to 126, 132 to 137, 197 to 202, 208 to 213, 219 to 224, 230 to 235, 241 to 246, 252 to 257, 303 to 308, 314 to 319, 325 to 330, 336 to 341, 347 to 352, 358 to 363, 421 to 426, 432 to 437, 443 to 448, 454 to 459, 465 to 470, 475 to 480, 514 to 519, 525 to 530, 536 to 541, 547 to 552, 558 to 563, 569 to 574. More preferably wherein the first antibody comprises CDR sequences comprising any one or more of SEQ ID NO: 055, 137, 257, 363, 480 or 574, and the second antibody comprises CDR sequences comprising any one or more of SEQ ID NO: 651, 652, 653, 654, 655 or SEQ ID NO.: 656. Preferably wherein the composition is administered for prophylactic and/or therapeutic treatment.

In a preferred embodiment, a first antibody as disclosed herein is provided as a composition in combination with a second antibody, wherein the first antibody comprises a CDR sequence comprising any one of SEQ ID NO: 001 to 006, 012 to 017, 023 to 028, 033 to 038, 042 to 047, 050 to 055, 077 to 082, 088 to 093, 099 to 104, 110 to 115, 121 to 126, 132 to 137, 197 to 202, 208 to 213, 219 to 224, 230 to 235, 241 to 246, 252 to 257, 303 to 308, 314 to 319, 325 to 330, 336 to 341, 347 to 352, 358 to 363, 421 to 426, 432 to 437, 443 to 448, 454 to 459, 465 to 470, 475 to 480, 514 to 519, 525 to 530, 536 to 541, 547 to 552, 558 to 563, 569 to 574. More preferably wherein the first antibody comprises CDR sequences comprising any one or more of SEQ ID NO: 055, 137, 257, 363, 480 or 574, and the second antibody comprises CDR sequences comprising any one or more of SEQ ID NO: 657, 658, 659, 660, 661 or SEQ ID NO.: 662. Preferably wherein the composition is administered for prophylactic and/or therapeutic treatment.

Preferably, the composition of the invention is a water-based composition such as an aqueous liquid. Preferably, the composition of the invention further comprises one or more salts, for example sodium chloride. The composition of the invention may further comprise one or more buffering agents, for example sodium acetate. The composition of the invention may further comprise one or more carbohydrates, such as sucrose, or other active ingredients, such as other antibodies, neuraminidase inhibitors, endonuclease inhibitors, and adjuvants such as oils, cytokines, emulsifiers, or combinations thereof. The pH of the composition of the invention can be between 4 and 8, more preferably the pH is around 5.5. Preferably the pH is around 7.4.

### Mucosal Composition

The term ‘mucosal composition’, as used herein, refers to any pharmaceutical formulation that may be topically applied to a mucosal surface and delays or resists the mucosal flushing or removal action.

The term ‘mucosal composition’, may be used in reference to a route of administration in which an antibody, as disclosed herein, is provided into the nasal cavity, oropharyngeal region or upper respiratory tract, preferably through the nostrils, as part of a prophylactic and/or therapeutic treatment as disclosed herein.

### Mucosally

The mucosa comprises membranes that line cavities in the human body, covering internal organs, and consist of one or more layers of epithelial cells and loose connective tissue, which may produce mucus. The term ‘mucosally’, as used herein, may also be referred to as ‘mucosal administration’, and includes reference to a route of administration in which a drug is prophylactically and/or therapeutically provided to the mucosa, for example, mucosa found in the nose, mouth, lungs, vagina, rectum or stomach. The mucous membrane lubricates and protects these organs and cavities from abrasive particles and bodily fluids, as well as invasive pathogens.

Preferably, the antibodies of the invention are administered mucosally.

### Intranasally

The term ‘intranasally’, as used herein, may also be referred to as ‘nasal administration’, and includes reference to a route of administration in which a drug is provided into the upper respiratory tract and/or lower respiratory tract, preferably through the nostrils, as part of a prophylactic and/or therapeutic treatment as disclosed herein. Preferably, the administration provides for drug in the nasal cavity. The back section of the nasal cavity is also referred to as the pharynx. Nasal administration preferably provides for delivery of antibodies as disclosed herein in the mucous membrane lining the nasal cavity. Intranasal administration can be performed using, for instance, a nasal spray or nose drops. In some embodiments, the drug is delivered to the nasal cavity via the oral route. For example, RetroNose uses a breath-actuated pressurised metered-dose inhaler (pMDI) to administer drugs through the buccal cavity during the nasal expiratory phase. Such methods allow the drug particles to enter the nasal cavities through the pharynx.

Preferably, the antibodies of the invention are administered intranasally.

With intranasal administration, a drug is provided through the nostrils to the upper respiratory tract as part of a prophylactic and/or therapeutic treatment as disclosed herein. Preferably, the administration provides for drug in the nasal cavity. Nasal administration can either be a form of topical administration or systemic administration, as the drugs thus locally delivered can go on to have either local or systemic effects. In the present case, with antibodies as disclosed herein, nasal administration is preferably a form of topical administration.

Intranasal administration as disclosed herein may be done using a medicament in liquid form, preferably in the form of drops or nasal spray. The aqueous liquid may comprise adjuvants. These adjuvants may for example be salts, oils, cytokines, emulsifiers, buffering agents, carbohydrates and combinations thereof. Intranasal administration may also be done using a medicament in solid form, such as powders.

### Oral Inhalation

The term ‘oral inhalation’, as used herein, may also be referred to as ‘mouth inhalation’, and includes reference to a route of administration in which a drug is provided through the mouth to the upper and/or lower respiratory tract such as lungs, as part of a prophylactic and/or therapeutic treatment of the invention. Oral inhalation may for example be applied for drugs in their powdered form and drugs in the form of liquid droplets or aerosols.

Preferably, the antibodies of the invention are administered by oral inhalation.

With oral inhalation, a drug is provided through the mouth to the respiratory tract, preferably lower respiratory tract such as lungs, as part of a prophylactic and/or therapeutic treatment of the invention. As described further herein, oral inhalation also includes nasal drug delivery (also referred to as nasal drug delivery via the oral route).

Oral inhalation may for example be applied for drugs in their powdered form and drugs in the form of liquid droplets or aerosols. Oral inhalation may include the use of an inhaler. The inhaler may be involved in the achievement of the dose that was determined. The drug that is administered by oral inhalation may reach the lung but may also partially be cleared out by exhalation.

Administration by oral inhalation as disclosed herein may be done using a medicament comprising aerosols in powdered (solid) or liquid form. Powdered aerosols comprising particles smaller than 3 μm in diameter will primarily reach the respiratory region of the lung, and will therefore be absorbed better than larger particles. The medicament may comprise adjuvants. These adjuvants may for example be salts, oils, cytokines, emulsifiers, buffering agents, carbohydrates and combinations thereof.

### Oropharyngeal Administration

The term ‘oropharyngeal administration’, as used herein, may also be referred to as delivery to the part of the pharynx that lies between the soft palate and the hyoid bone and includes reference to a route of administration in which a drug is provided via either the mouth or nasal passages as part of a prophylactic and/or therapeutic treatment. Oropharyngeal administration may, for example, be used for drugs in their powdered form and drugs in the form of liquid droplets or aerosols.

Preferably, the antibodies of the invention are administered by oropharyngeal administration.

### Prior to

The term ‘prior to’, as used herein, includes reference to the administration of an antibody before an individual has been exposed to, or is infected with, a coronavirus.

Preferably, the antibody as disclosed is administered to an individual up to 24 hours prior to coronavirus exposure, for example between zero and 24 hours before the individual has been exposed to said coronavirus.

Preferably, the antibody as disclosed is administered to an individual up to 48 hours prior to coronavirus exposure, for example between zero and 48 hours before the individual has been exposed to said coronavirus.

In a preferred embodiment, the antibody is administered 2 days or more prior to coronavirus exposure. In a preferred embodiment, the antibody is administered 3 days or more prior to coronavirus exposure. In a preferred embodiment, the antibody is administered 4 days, or more prior to coronavirus exposure. In a preferred embodiment, the antibody is administered 5 days or more prior to coronavirus exposure. In a preferred embodiment, the antibody is administered 6 days or more prior to coronavirus exposure. In a preferred embodiment, the antibody is administered 7 days or more prior to coronavirus exposure.

### Dosage

The term ‘dosage’ as used herein, refers to the amount of antibody to be given at a particular time (e.g., over the course of a 24-hour, 12-hour, 30-minute period, etc.). A dose refers to a single dosing episode, whether the dose is a unit dosage form or multiple unit dosage forms taken together (e.g., ingestion of two or more pills, receiving two or more nasal administrations). A dosage includes reference to a pharmaceutical dosage form wherein the medicament is packaged for administration as, e.g., a single-unit dose or multiple-unit dose. A dosage may also be administered as, e.g., one or more drops of an antibody-comprising composition (e.g., nasal drops) or one or more sprays of an antibody-comprising composition (e.g., nasal sprays).

Preferably, a suitable dosage of an antibody as disclosed herein contains a dose of between 0.01 mg and 20 mg, preferably 0.1 mg and 15 mg, more preferably around 0.5 mg and 10 mg or even around 1 mg, e.g. when the dosage is for intranasal administration. Such dosages are also referred to as “flat dosages” or “nominal dosages” in contrast to dosages based on the weight of the patient. Flat dosages have the advantage that the medicament can be packaged in a single-unit dose, for example. A single dose of an antibody according to the present invention can provide protection from coronavirus infection for several days and may be provided “on demand” or “as needed”. For example, an individual may administer antibody before leaving the house or before coming into contact with other individuals.

In order to provide long-lasting protection, the antibody may be administered on a regular basis. For example, the antibody is administered once, or at least once per month. In a preferred embodiment, the antibody is administered once, or at least once per week, e.g., twice weekly. In a preferred embodiment, the antibody is administered once, or at least once per day. As is clear to a skilled person, less antibody may be administered when the antibody is administered more frequently (e.g., daily). In a preferred embodiment, between 0.01 mg to 20 mg of antibody is administered per week (e.g., once or twice weekly or daily). In an exemplary embodiment, between 0.1 mg and 25 mg is administered daily (i.e. between 0.7 mg and 175 mg per week). In an exemplary embodiment, 0.5 mg to 3.5 mg is administered daily (i.e. between 3.5 mg and 24.5 mg per week).

The invention also provides a composition formulated for intranasal administration and/or oral inhalation comprising an antibody as disclosed herein, preferably in a single dose unit between 0.1 mg to 20 mg, preferably 0.5 mg to 15 mg or preferably 1 mg to 12.5 mg.

### Use in a Method for Treatment

The present invention relates inter alia to an antibody as disclosed herein, for use in a method for treatment of coronavirus infection in an individual, more specifically the antibody can be used in a method for the prophylactic and/or therapeutic treatment of coronavirus infection in an individual.

### Complementarity-Determining Regions (CDRs)

Preferably, the CDR regions are identified according to Kabat et al. (1991) as described in Sequences of Proteins of Immunological Interest.

In a preferred embodiment, the binding interaction of the binding molecules, preferably the antibody, and the stem helix is mediated exclusively by light and heavy chain variable sequences.

The antibody as disclosed herein may be capable of specifically binding to a coronavirus that is in attenuated or inactivated form or that is viable, and/or in an infective form.

The antibody as disclosed herein is also capable of specifically binding to one or more fragments of the coronavirus.

An antibody as disclosed herein comprises a preferred heavy chain CDR1 sequence comprising any one of the following SEQ ID NO: 001 to 006, 012 to 017, 023 to 028, 033 to 038, 042 to 047, 050 to 055. A CDR comprising the amino acid sequence SEQ ID NO: 055 is especially preferred.

An antibody as disclosed herein comprises a preferred heavy chain CDR2 sequence comprising any one of the following SEQ ID NO: 077 to 082, 088 to 093, 099 to 104, 110 to 115, 121 to 126, 132 to 137. A CDR comprising the amino acid sequence SEQ ID NO: 137 is especially preferred.

An antibody as disclosed herein comprises a preferred heavy chain CDR3 sequence comprising any one of the following SEQ ID NO: 197 to 202, 208 to 213, 219 to 224, 230 to 235, 241 to 246, 252 to 257. A CDR comprising the amino acid sequence SEQ ID NO: 257 is especially preferred.

An antibody as disclosed herein comprises a preferred light chain CDR1 sequence comprising any one of the following SEQ ID NO: 303 to 308, 314 to 319, 325 to 330, 336 to 341, 347 to 352, 358 to 363. A CDR comprising the amino acid sequence SEQ ID NO: 363 is especially preferred.

An antibody as disclosed herein comprises a preferred light chain CDR2 sequence comprising any one of the following SEQ ID NO: 421 to 426, 432 to 437, 443 to 448, 454 to 459, 465 to 470, 475 to 480. A CDR comprising the amino acid sequence SEQ ID NO: 480 is especially preferred.

An antibody as disclosed herein comprises a preferred light chain CDR3 sequence comprising any one of the following SEQ ID NO: 514 to 519, 525 to 530, 536 to 541, 547 to 552, 558 to 563, 569 to 574. A CDR comprising the amino acid sequence SEQ ID NO: 574 is especially preferred.

An antibody as disclosed herein comprises a heavy chain variable domain that further comprises at least a heavy chain framework region FRI of SEQ ID NO: 631.

An antibody as disclosed herein comprises a heavy chain variable domain that further comprises at least a heavy chain framework region FR2 of SEQ ID NO: 632.

An antibody as disclosed herein comprises a heavy chain variable domain that further comprises at least a heavy chain framework region FR3 of SEQ ID NO: 633.

An antibody as disclosed herein comprises a heavy chain variable domain that further comprises at least a heavy chain framework region FR4 of SEQ ID NO: 634.

In a preferred embodiment, the heavy chain variable domain of said antibody comprises—a heavy chain framework region FR1 of SEQ ID NO: 631, a heavy chain framework region FR2 of SEQ ID NO: 632, a heavy chain framework region FR3 of SEQ ID NO: 633, and/or a heavy chain framework region FR4 of SEQ ID NO: 634, preferably all said heavy chain heavy chain framework regions FR1 to FR4.

An antibody as disclosed herein comprises a heavy chain variable domain that further comprises at least a light chain framework region FR1 of SEQ ID NO: 635.

An antibody as disclosed herein comprises a heavy chain variable domain that further comprises at least a light chain framework region FR2 of SEQ ID NO: 636.

An antibody as disclosed herein comprises a heavy chain variable domain that further comprises at least a light chain framework region FR3 of SEQ ID NO: 637.

An antibody as disclosed herein comprises a heavy chain variable domain that further comprises at least a light chain framework region FR4 of SEQ ID NO: 638.

In a preferred embodiment, the light chain variable domain of said antibody comprises—a light chain framework region FR1 of SEQ ID NO: 635, a light chain framework region FR2 of SEQ ID NO: 636, a light chain framework region FR3 of SEQ ID NO: 637, and/or a light chain framework region FR4 of SEQ ID NO: 638, preferably all said light chain framework regions are FR1 to FR4.

Most preferably, an antibody as disclosed herein comprises a heavy chain variable domain having the sequence of SEQ ID NO: 629 and/or a light chain variable domain having the sequence of SEQ ID NO: 630.

In treatment methods of the invention, the heavy chain variable domain (VH) of the antibody is preferably indicated as SEQ ID NO:629. In treatment methods or compositions of the invention, the light chain variable domain (VL) of the antibody is preferably indicated as SEQ ID NO:630. In treatment methods or compositions of the invention, the antibody can be an antibody as disclosed herein, or a binding molecule comprising an antigen-binding domain such as a variable domain (V) thereof.

Preferably, an antibody as disclosed herein comprises a heavy chain variable domain having the sequence of SEQ ID NO:629 having at most 15, preferably 0, 1, 2, 3, 4, 5, 6, 7, 8, 9 or 10, more preferably having 0, 1, 2, 3, 4 or 5 amino acid insertions, deletions, substitutions.

Preferably, an antibody as disclosed herein comprises a light chain variable domain having the sequence of SEQ ID NO:630 having at most 15, preferably 0, 1, 2, 3, 4, 5, 6, 7, 8, 9 or 10, more preferably having 0, 1, 2, 3, 4 or 5 amino acid insertions, deletions, substitutions.

Preferably, said amino acid insertions, deletions, substitutions in respect of either the heavy chain variable domain and/or the light chain variable domain are not in the CDRs.

### SEQUENCES

CDRs and framework regions defined herein are based on the Kabat numbering scheme as described in Sequences of Proteins of Immunological Interest (Kabat et al., 1991).

| Heavy chain CDR1 region | |
|---|---|
| (SEQ ID NO: 001 to 076) | |
| SEQ ID NO.: 001 | GFTFSYFYLHWVRQA |
| SEQ ID NO.: 002 | FTFSYFYLHWVRQA |
| SEQ ID NO.: 003 | TFSYFYLHWVRQA |
| SEQ ID NO.: 004 | FSYFYLHWVRQA |
| SEQ ID NO.: 005 | SYFYLHWVRQA |
| SEQ ID NO.: 006 | YFYLHWVRQA |
| SEQ ID NO.: 007 | FYLHWVRQA |
| SEQ ID NO.: 008 | YLHWVRQA |
| SEQ ID NO.: 009 | LHWVRQA |
| SEQ ID NO.: 010 | HWVRQA |
| SEQ ID NO.: 011 | WVRQA |
| SEQ ID NO.: 012 | GFTFSYFYLHWVRQ |
| SEQ ID NO.: 013 | FTFSYFYLHWVRQ |
| SEQ ID NO.: 014 | TFSYFYLHWVRQ |
| SEQ ID NO.: 015 | FSYFYLHWVRQ |
| SEQ ID NO.: 016 | SYFYLHWVRQ |
| SEQ ID NO.: 017 | YFYLHWVRQ |
| SEQ ID NO.: 018 | FYLHWVRQ |
| SEQ ID NO.: 019 | YLHWVRQ |
| SEQ ID NO.: 020 | LHWVRQ |
| SEQ ID NO.: 021 | HWVRQ |
| SEQ ID NO.: 022 | WVRQ |
| SEQ ID NO.: 023 | GFTFSYFYLHWVR |
| SEQ ID NO.: 024 | FTFSYFYLHWVR |
| SEQ ID NO.: 025 | TFSYFYLHWVR |
| SEQ ID NO.: 026 | FSYFYLHWVR |
| SEQ ID NO.: 027 | SYFYLHWVR |
| SEQ ID NO.: 028 | YFYLHWVR |
| SEQ ID NO.: 029 | FYLHWVR |
| SEQ ID NO.: 030 | YLHWVR |
| SEQ ID NO.: 031 | LHWVR |
| SEQ ID NO.: 032 | HWVR |
| SEQ ID NO.: 033 | GFTFSYFYLHWV |
| SEQ ID NO.: 034 | FTFSYFYLHWV |
| SEQ ID NO.: 035 | TFSYFYLHWV |
| SEQ ID NO.: 036 | FSYFYLHWV |
| SEQ ID NO.: 037 | SYFYLHWV |
| SEQ ID NO.: 038 | YFYLHWV |
| SEQ ID NO.: 039 | FYLHWV |
| SEQ ID NO.: 040 | YLHWV |
| SEQ ID NO.: 041 | LHWV |
| SEQ ID NO.: 042 | GFTFSYFYLHW |
| SEQ ID NO.: 043 | FTFSYFYLHW |
| SEQ ID NO.: 044 | TFSYFYLHW |
| SEQ ID NO.: 045 | FSYFYLHW |
| SEQ ID NO.: 046 | SYFYLHW |
| SEQ ID NO.: 047 | YFYLHW |
| SEQ ID NO.: 048 | FYLHW |
| SEQ ID NO.: 049 | YLHW |
| SEQ ID NO.: 050 | GFTFSYFYLH |
| SEQ ID NO.: 051 | FTFSYFYLH |
| SEQ ID NO.: 052 | TFSYFYLH |
| SEQ ID NO.: 053 | FSYFYLH |
| SEQ ID NO.: 054 | SYFYLH |
| SEQ ID NO.: 055 | YFYLH |
| SEQ ID NO.: 056 | FYLH |
| SEQ ID NO.: 057 | GFTFSYFYL |
| SEQ ID NO.: 058 | FTFSYFYL |
| SEQ ID NO.: 059 | TFSYFYL |
| SEQ ID NO.: 060 | FSYFYL |
| SEQ ID NO.: 061 | SYFYL |
| SEQ ID NO.: 062 | YFYL |
| SEQ ID NO.: 063 | GFTFSYFY |
| SEQ ID NO.: 064 | FTFSYFY |
| SEQ ID NO.: 065 | TFSYFY |
| SEQ ID NO.: 066 | FSYFY |
| SEQ ID NO.: 067 | SYFY |
| SEQ ID NO.: 068 | GFTFSYF |
| SEQ ID NO.: 069 | FTFSYF |
| SEQ ID NO.: 070 | TFSYF |
| SEQ ID NO.: 071 | FSYF |
| SEQ ID NO.: 072 | GFTFSY |
| SEQ ID NO.: 073 | FTFSY |
| SEQ ID NO.: 074 | TFSY |
| SEQ ID NO.: 075 | GFTFS |
| SEQ ID NO.: 076 | FTFS |
| Heavy chain CDR2 region | |
| (SEQ ID NO: 077 to 196) | |
| SEQ ID NO.: 077 | LEWMGIINPRGDGTRYAQKFQGRVTMT |
| SEQ ID NO.: 078 | EWMGIINPRGDGTRYAQKFQGRVTMT |
| SEQ ID NO.: 079 | WMGIINPRGDGTRYAQKFQGRVTMT |
| SEQ ID NO.: 080 | MGIINPRGDGTRYAQKFQGRVTMT |
| SEQ ID NO.: 081 | GIINPRGDGTRYAQKFQGRVTMT |
| SEQ ID NO.: 082 | IINPRGDGTRYAQKFQGRVTMT |
| SEQ ID NO.: 083 | INPRGDGTRYAQKFQGRVTMT |
| SEQ ID NO.: 084 | NPRGDGTRYAQKFQGRVTMT |
| SEQ ID NO.: 085 | PRGDGTRYAQKFQGRVTMT |
| SEQ ID NO.: 086 | RGDGTRYAQKFQGRVTMT |
| SEQ ID NO.: 087 | GDGTRYAQKFQGRVTMT |
| SEQ ID NO.: 088 | LEWMGIINPRGDGTRYAQKFQGRVTM |
| SEQ ID NO.: 089 | EWMGIINPRGDGTRYAQKFQGRVTM |
| SEQ ID NO.: 090 | WMGIINPRGDGTRYAQKFQGRVTM |
| SEQ ID NO.: 091 | MGIINPRGDGTRYAQKFQGRVTM |
| SEQ ID NO.: 092 | GIINPRGDGTRYAQKFQGRVTM |
| SEQ ID NO.: 093 | IINPRGDGTRYAQKFQGRVTM |
| SEQ ID NO.: 094 | INPRGDGTRYAQKFQGRVTM |
| SEQ ID NO.: 095 | NPRGDGTRYAQKFQGRVTM |
| SEQ ID NO.: 096 | PRGDGTRYAQKFQGRVTM |
| SEQ ID NO.: 097 | RGDGTRYAQKFQGRVTM |
| SEQ ID NO.: 098 | GDGTRYAQKFQGRVTM |
| SEQ ID NO.: 099 | LEWMGIINPRGDGTRYAQKFQGRVT |
| SEQ ID NO.: 100 | EWMGIINPRGDGTRYAQKFQGRVT |
| SEQ ID NO.: 101 | WMGIINPRGDGTRYAQKFQGRVT |
| SEQ ID NO.: 102 | MGIINPRGDGTRYAQKFQGRVT |
| SEQ ID NO.: 103 | GIINPRGDGTRYAQKFQGRVT |
| SEQ ID NO.: 104 | IINPRGDGTRYAQKFQGRVT |
| SEQ ID NO.: 105 | INPRGDGTRYAQKFQGRVT |
| SEQ ID NO.: 106 | NPRGDGTRYAQKFQGRVT |
| SEQ ID NO.: 107 | PRGDGTRYAQKFQGRVT |
| SEQ ID NO.: 108 | RGDGTRYAQKFQGRVT |
| SEQ ID NO.: 109 | GDGTRYAQKFQGRVT |
| SEQ ID NO.: 110 | LEWMGIINPRGDGTRYAQKFQGRV |
| SEQ ID NO.: 111 | EWMGIINPRGDGTRYAQKFQGRV |
| SEQ ID NO.: 112 | WMGIINPRGDGTRYAQKFQGRV |
| SEQ ID NO.: 113 | MGIINPRGDGTRYAQKFQGRV |
| SEQ ID NO.: 114 | GIINPRGDGTRYAQKFQGRV |
| SEQ ID NO.: 115 | IINPRGDGTRYAQKFQGRV |
| SEQ ID NO.: 116 | INPRGDGTRYAQKFQGRV |
| SEQ ID NO.: 117 | NPRGDGTRYAQKFQGRV |
| SEQ ID NO.: 118 | PRGDGTRYAQKFQGRV |
| SEQ ID NO.: 119 | RGDGTRYAQKFQGRV |
| SEQ ID NO.: 120 | GDGTRYAQKFQGRV |
| SEQ ID NO.: 121 | LEWMGIINPRGDGTRYAQKFQGR |
| SEQ ID NO.: 122 | EWMGIINPRGDGTRYAQKFQGR |
| SEQ ID NO.: 123 | WMGIINPRGDGTRYAQKFQGR |
| SEQ ID NO.: 124 | MGIINPRGDGTRYAQKFQGR |
| SEQ ID NO.: 125 | GIINPRGDGTRYAQKFQGR |
| SEQ ID NO.: 126 | IINPRGDGTRYAQKFQGR |
| SEQ ID NO.: 127 | INPRGDGTRYAQKFQGR |
| SEQ ID NO.: 128 | NPRGDGTRYAQKFQGR |
| SEQ ID NO.: 129 | PRGDGTRYAQKFQGR |
| SEQ ID NO.: 130 | RGDGTRYAQKFQGR |
| SEQ ID NO.: 131 | GDGTRYAQKFQGR |
| SEQ ID NO.: 132 | LEWMGIINPRGDGTRYAQKFQG |
| SEQ ID NO.: 133 | EWMGIINPRGDGTRYAQKFQG |
| SEQ ID NO.: 134 | WMGIINPRGDGTRYAQKFQG |
| SEQ ID NO.: 135 | MGIINPRGDGTRYAQKFQG |
| SEQ ID NO.: 136 | GIINPRGDGTRYAQKFQG |
| SEQ ID NO.: 137 | IINPRGDGTRYAQKFQG |
| SEQ ID NO.: 138 | INPRGDGTRYAQKFQG |
| SEQ ID NO.: 139 | NPRGDGTRYAQKFQG |
| SEQ ID NO.: 140 | PRGDGTRYAQKFQG |
| SEQ ID NO.: 141 | RGDGTRYAQKFQG |
| SEQ ID NO.: 142 | GDGTRYAQKFQG |
| SEQ ID NO.: 143 | LEWMGIINPRGDGTRYAQKFQ |
| SEQ ID NO.: 144 | EWMGIINPRGDGTRYAQKFQ |
| SEQ ID NO.: 145 | WMGIINPRGDGTRYAQKFQ |
| SEQ ID NO.: 146 | MGIINPRGDGTRYAQKFQ |
| SEQ ID NO.: 147 | GIINPRGDGTRYAQKFQ |
| SEQ ID NO.: 148 | IINPRGDGTRYAQKFQ |
| SEQ ID NO.: 149 | INPRGDGTRYAQKFQ |
| SEQ ID NO.: 150 | NPRGDGTRYAQKFQ |
| SEQ ID NO.: 151 | PRGDGTRYAQKFQ |
| SEQ ID NO.: 152 | RGDGTRYAQKFQ |
| SEQ ID NO.: 153 | GDGTRYAQKFQ |
| SEQ ID NO.: 154 | LEWMGIINPRGDGTRYAQKF |
| SEQ ID NO.: 155 | EWMGIINPRGDGTRYAQKF |
| SEQ ID NO.: 156 | WMGIINPRGDGTRYAQKF |
| SEQ ID NO.: 157 | MGIINPRGDGTRYAQKF |
| SEQ ID NO.: 158 | GIINPRGDGTRYAQKF |
| SEQ ID NO.: 159 | IINPRGDGTRYAQKF |
| SEQ ID NO.: 160 | INPRGDGTRYAQKF |
| SEQ ID NO.: 161 | NPRGDGTRYAQKF |
| SEQ ID NO.: 162 | PRGDGTRYAQKF |
| SEQ ID NO.: 163 | RGDGTRYAQKF |
| SEQ ID NO.: 164 | GDGTRYAQKF |
| SEQ ID NO.: 165 | LEWMGIINPRGDGTRYAQK |
| SEQ ID NO.: 166 | EWMGIINPRGDGTRYAQK |
| SEQ ID NO.: 167 | WMGIINPRGDGTRYAQK |
| SEQ ID NO.: 168 | MGIINPRGDGTRYAQK |
| SEQ ID NO.: 169 | GIINPRGDGTRYAQK |
| SEQ ID NO.: 170 | IINPRGDGTRYAQK |
| SEQ ID NO.: 171 | INPRGDGTRYAQK |
| SEQ ID NO.: 172 | NPRGDGTRYAQK |
| SEQ ID NO.: 173 | PRGDGTRYAQK |
| SEQ ID NO.: 174 | RGDGTRYAQK |
| SEQ ID NO.: 175 | GDGTRYAQK |
| SEQ ID NO.: 176 | LEWMGIINPRGDGTRYAQ |
| SEQ ID NO.: 177 | EWMGIINPRGDGTRYAQ |
| SEQ ID NO.: 178 | WMGIINPRGDGTRYAQ |
| SEQ ID NO.: 179 | MGIINPRGDGTRYAQ |
| SEQ ID NO.: 180 | GIINPRGDGTRYAQ |
| SEQ ID NO.: 181 | IINPRGDGTRYAQ |
| SEQ ID NO.: 182 | INPRGDGTRYAQ |
| SEQ ID NO.: 183 | NPRGDGTRYAQ |
| SEQ ID NO.: 184 | PRGDGTRYAQ |
| SEQ ID NO.: 185 | RGDGTRYAQ |
| SEQ ID NO.: 186 | GDGTRYAQ |
| SEQ ID NO.: 187 | LEWMGIINPRGDGTRYA |
| SEQ ID NO.: 188 | EWMGIINPRGDGTRYA |
| SEQ ID NO.: 189 | WMGIINPRGDGTRYA |
| SEQ ID NO.: 190 | MGIINPRGDGTRYA |
| SEQ ID NO.: 191 | GIINPRGDGTRYA |
| SEQ ID NO.: 192 | IINPRGDGTRYA |
| SEQ ID NO.: 193 | INPRGDGTRYA |
| SEQ ID NO.: 194 | NPRGDGTRYA |
| SEQ ID NO.: 195 | PRGDGTRYA |
| SEQ ID NO.: 196 | RGDGTRYA |
| Heavy chain CDR3 region | |
| (SEQ ID NO: 197 to 302) | |
| SEQ ID NO.: 197 | YYCARGADHGAFDIWGQGT |
| SEQ ID NO.: 198 | YCARGADHGAFDIWGQGT |
| SEQ ID NO.: 199 | CARGADHGAFDIWGQGT |
| SEQ ID NO.: 200 | ARGADHGAFDIWGQGT |
| SEQ ID NO.: 201 | RGADHGAFDIWGQGT |
| SEQ ID NO.: 202 | GADHGAFDIWGQGT |
| SEQ ID NO.: 203 | ADHGAFDIWGQGT |
| SEQ ID NO.: 204 | DHGAFDIWGQGT |
| SEQ ID NO.: 205 | HGAFDIWGQGT |
| SEQ ID NO.: 206 | GAFDIWGQGT |
| SEQ ID NO.: 207 | AFDIWGQGT |
| SEQ ID NO.: 208 | YYCARGADHGAFDIWGQG |
| SEQ ID NO.: 209 | YCARGADHGAFDIWGQG |
| SEQ ID NO.: 210 | CARGADHGAFDIWGQG |
| SEQ ID NO.: 211 | ARGADHGAFDIWGQG |
| SEQ ID NO.: 212 | RGADHGAFDIWGQG |
| SEQ ID NO.: 213 | GADHGAFDIWGQG |
| SEQ ID NO.: 214 | ADHGAFDIWGQG |
| SEQ ID NO.: 215 | DHGAFDIWGQG |
| SEQ ID NO.: 216 | HGAFDIWGQG |
| SEQ ID NO.: 217 | GAFDIWGQG |
| SEQ ID NO.: 218 | AFDIWGQG |
| SEQ ID NO.: 219 | YYCARGADHGAFDIWGQ |
| SEQ ID NO.: 220 | YCARGADHGAFDIWGQ |
| SEQ ID NO.: 221 | CARGADHGAFDIWGQ |
| SEQ ID NO.: 222 | ARGADHGAFDIWGQ |
| SEQ ID NO.: 223 | RGADHGAFDIWGQ |
| SEQ ID NO.: 224 | GADHGAFDIWGQ |
| SEQ ID NO.: 225 | ADHGAFDIWGQ |
| SEQ ID NO.: 226 | DHGAFDIWGQ |
| SEQ ID NO.: 227 | HGAFDIWGQ |
| SEQ ID NO.: 228 | GAFDIWGQ |
| SEQ ID NO.: 229 | AFDIWGQ |
| SEQ ID NO.: 230 | YYCARGADHGAFDIWG |
| SEQ ID NO.: 231 | YCARGADHGAFDIWG |
| SEQ ID NO.: 232 | CARGADHGAFDIWG |
| SEQ ID NO.: 233 | ARGADHGAFDIWG |
| SEQ ID NO.: 234 | RGADHGAFDIWG |
| SEQ ID NO.: 235 | GADHGAFDIWG |
| SEQ ID NO.: 236 | ADHGAFDIWG |
| SEQ ID NO.: 237 | DHGAFDIWG |
| SEQ ID NO.: 238 | HGAFDIWG |
| SEQ ID NO.: 239 | GAFDIWG |
| SEQ ID NO.: 240 | AFDIWG |
| SEQ ID NO.: 241 | YYCARGADHGAFDIW |
| SEQ ID NO.: 242 | YCARGADHGAFDIW |
| SEQ ID NO.: 243 | CARGADHGAFDIW |
| SEQ ID NO.: 244 | ARGADHGAFDIW |
| SEQ ID NO.: 245 | RGADHGAFDIW |
| SEQ ID NO.: 246 | GADHGAFDIW |
| SEQ ID NO.: 247 | ADHGAFDIW |
| SEQ ID NO.: 248 | DHGAFDIW |
| SEQ ID NO.: 249 | HGAFDIW |
| SEQ ID NO.: 250 | GAFDIW |
| SEQ ID NO.: 251 | AFDIW |
| SEQ ID NO.: 252 | YYCARGADHGAFDI |
| SEQ ID NO.: 253 | YCARGADHGAFDI |
| SEQ ID NO.: 254 | CARGADHGAFDI |
| SEQ ID NO.: 255 | ARGADHGAFDI |
| SEQ ID NO.: 256 | RGADHGAFDI |
| SEQ ID NO.: 257 | GADHGAFDI |
| SEQ ID NO.: 258 | ADHGAFDI |
| SEQ ID NO.: 259 | DHGAFDI |
| SEQ ID NO.: 260 | HGAFDI |
| SEQ ID NO.: 261 | GAFDI |
| SEQ ID NO.: 262 | AFDI |
| SEQ ID NO.: 263 | YYCARGADHGAFD |
| SEQ ID NO.: 264 | YCARGADHGAFD |
| SEQ ID NO.: 265 | CARGADHGAFD |
| SEQ ID NO.: 266 | ARGADHGAFD |
| SEQ ID NO.: 267 | RGADHGAFD |
| SEQ ID NO.: 268 | GADHGAFD |
| SEQ ID NO.: 269 | ADHGAFD |
| SEQ ID NO.: 270 | DHGAFD |
| SEQ ID NO.: 271 | HGAFD |
| SEQ ID NO.: 272 | GAFD |
| SEQ ID NO.: 273 | YYCARGADHGAF |
| SEQ ID NO.: 274 | YCARGADHGAF |
| SEQ ID NO.: 275 | CARGADHGAF |
| SEQ ID NO.: 276 | ARGADHGAF |
| SEQ ID NO.: 277 | RGADHGAF |
| SEQ ID NO.: 278 | GADHGAF |
| SEQ ID NO.: 279 | ADHGAF |
| SEQ ID NO.: 280 | DHGAF |
| SEQ ID NO.: 281 | HGAF |
| SEQ ID NO.: 282 | YYCARGADHGA |
| SEQ ID NO.: 283 | YCARGADHGA |
| SEQ ID NO.: 284 | CARGADHGA |
| SEQ ID NO.: 285 | ARGADHGA |
| SEQ ID NO.: 286 | RGADHGA |
| SEQ ID NO.: 287 | GADHGA |
| SEQ ID NO.: 288 | ADHGA |
| SEQ ID NO.: 289 | DHGA |
| SEQ ID NO.: 290 | YYCARGADHG |
| SEQ ID NO.: 291 | YCARGADHG |
| SEQ ID NO.: 292 | CARGADHG |
| SEQ ID NO.: 293 | ARGADHG |
| SEQ ID NO.: 294 | RGADHG |
| SEQ ID NO.: 295 | GADHG |
| SEQ ID NO.: 296 | ADHG |
| SEQ ID NO.: 297 | YYCARGADH |
| SEQ ID NO.: 298 | YCARGADH |
| SEQ ID NO.: 299 | CARGADH |
| SEQ ID NO.: 300 | ARGADH |
| SEQ ID NO.: 301 | RGADH |
| SEQ ID NO.: 302 | GADH |
| Light chain CDR1 region | |
| (SEQ ID NO: 303 to 420) | |
| SEQ ID NO.: 303 | ATLSCRASQSVRRNYFAWYQQK |
| SEQ ID NO.: 304 | TLSCRASQSVRRNYFAWYQQK |
| SEQ ID NO.: 305 | LSCRASQSVRRNYFAWYQQK |
| SEQ ID NO.: 306 | SCRASQSVRRNYFAWYQQK |
| SEQ ID NO.: 307 | CRASQSVRRNYFAWYQQK |
| SEQ ID NO.: 308 | RASQSVRRNYFAWYQQK |
| SEQ ID NO.: 309 | ASQSVRRNYFAWYQQK |
| SEQ ID NO.: 310 | SQSVRRNYFAWYQQK |
| SEQ ID NO.: 311 | QSVRRNYFAWYQQK |
| SEQ ID NO.: 312 | SVRRNYFAWYQQK |
| SEQ ID NO.: 313 | VRRNYFAWYQQK |
| SEQ ID NO.: 314 | ATLSCRASQSVRRNYFAWYQQ |
| SEQ ID NO.: 315 | TLSCRASQSVRRNYFAWYQQ |
| SEQ ID NO.: 316 | LSCRASQSVRRNYFAWYQQ |
| SEQ ID NO.: 317 | SCRASQSVRRNYFAWYQQ |
| SEQ ID NO.: 318 | CRASQSVRRNYFAWYQQ |
| SEQ ID NO.: 319 | RASQSVRRNYFAWYQQ |
| SEQ ID NO.: 320 | ASQSVRRNYFAWYQQ |
| SEQ ID NO.: 321 | SQSVRRNYFAWYQQ |
| SEQ ID NO.: 322 | QSVRRNYFAWYQQ |
| SEQ ID NO.: 323 | SVRRNYFAWYQQ |
| SEQ ID NO.: 324 | VRRNYFAWYQQ |
| SEQ ID NO.: 325 | ATLSCRASQSVRRNYFAWYQ |
| SEQ ID NO.: 326 | TLSCRASQSVRRNYFAWYQ |
| SEQ ID NO.: 327 | LSCRASQSVRRNYFAWYQ |
| SEQ ID NO.: 328 | SCRASQSVRRNYFAWYQ |
| SEQ ID NO.: 329 | CRASQSVRRNYFAWYQ |
| SEQ ID NO.: 330 | RASQSVRRNYFAWYQ |
| SEQ ID NO.: 331 | ASQSVRRNYFAWYQ |
| SEQ ID NO.: 332 | SQSVRRNYFAWYQ |
| SEQ ID NO.: 333 | QSVRRNYFAWYQ |
| SEQ ID NO.: 334 | SVRRNYFAWYQ |
| SEQ ID NO.: 335 | VRRNYFAWYQ |
| SEQ ID NO.: 336 | ATLSCRASQSVRRNYFAWY |
| SEQ ID NO.: 337 | TLSCRASQSVRRNYFAWY |
| SEQ ID NO.: 338 | LSCRASQSVRRNYFAWY |
| SEQ ID NO.: 339 | SCRASQSVRRNYFAWY |
| SEQ ID NO.: 340 | CRASQSVRRNYFAWY |
| SEQ ID NO.: 341 | RASQSVRRNYFAWY |
| SEQ ID NO.: 342 | ASQSVRRNYFAWY |
| SEQ ID NO.: 343 | SQSVRRNYFAWY |
| SEQ ID NO.: 344 | QSVRRNYFAWY |
| SEQ ID NO.: 345 | SVRRNYFAWY |
| SEQ ID NO.: 346 | VRRNYFAWY |
| SEQ ID NO.: 347 | ATLSCRASQSVRRNYFAW |
| SEQ ID NO.: 348 | TLSCRASQSVRRNYFAW |
| SEQ ID NO.: 349 | LSCRASQSVRRNYFAW |
| SEQ ID NO.: 350 | SCRASQSVRRNYFAW |
| SEQ ID NO.: 351 | CRASQSVRRNYFAW |
| SEQ ID NO.: 352 | RASQSVRRNYFAW |
| SEQ ID NO.: 353 | ASQSVRRNYFAW |
| SEQ ID NO.: 354 | SQSVRRNYFAW |
| SEQ ID NO.: 355 | QSVRRNYFAW |
| SEQ ID NO.: 356 | SVRRNYFAW |
| SEQ ID NO.: 357 | VRRNYFAW |
| SEQ ID NO.: 358 | ATLSCRASQSVRRNYFA |
| SEQ ID NO.: 359 | TLSCRASQSVRRNYFA |
| SEQ ID NO.: 360 | LSCRASQSVRRNYFA |
| SEQ ID NO.: 361 | SCRASQSVRRNYFA |
| SEQ ID NO.: 362 | CRASQSVRRNYFA |
| SEQ ID NO.: 363 | RASQSVRRNYFA |
| SEQ ID NO.: 364 | ASQSVRRNYFA |
| SEQ ID NO.: 365 | SQSVRRNYFA |
| SEQ ID NO.: 366 | QSVRRNYFA |
| SEQ ID NO.: 367 | SVRRNYFA |
| SEQ ID NO.: 368 | VRRNYFA |
| SEQ ID NO.: 369 | ATLSCRASQSVRRNYF |
| SEQ ID NO.: 370 | TLSCRASQSVRRNYF |
| SEQ ID NO.: 371 | LSCRASQSVRRNYF |
| SEQ ID NO.: 372 | SCRASQSVRRNYF |
| SEQ ID NO.: 373 | CRASQSVRRNYF |
| SEQ ID NO.: 374 | RASQSVRRNYF |
| SEQ ID NO.: 375 | ASQSVRRNYF |
| SEQ ID NO.: 376 | SQSVRRNYF |
| SEQ ID NO.: 377 | QSVRRNYF |
| SEQ ID NO.: 378 | SVRRNYF |
| SEQ ID NO.: 379 | VRRNYF |
| SEQ ID NO.: 380 | ATLSCRASQSVRRNY |
| SEQ ID NO.: 381 | TLSCRASQSVRRNY |
| SEQ ID NO.: 382 | LSCRASQSVRRNY |
| SEQ ID NO.: 383 | SCRASQSVRRNY |
| SEQ ID NO.: 384 | CRASQSVRRNY |
| SEQ ID NO.: 385 | RASQSVRRNY |
| SEQ ID NO.: 386 | ASQSVRRNY |
| SEQ ID NO.: 387 | SQSVRRNY |
| SEQ ID NO.: 388 | QSVRRNY |
| SEQ ID NO.: 389 | SVRRNY |
| SEQ ID NO.: 390 | VRRNY |
| SEQ ID NO.: 391 | ATLSCRASQSVRRN |
| SEQ ID NO.: 392 | TLSCRASQSVRRN |
| SEQ ID NO.: 393 | LSCRASQSVRRN |
| SEQ ID NO.: 394 | SCRASQSVRRN |
| SEQ ID NO.: 395 | CRASQSVRRN |
| SEQ ID NO.: 396 | RASQSVRRN |
| SEQ ID NO.: 397 | ASQSVRRN |
| SEQ ID NO.: 398 | SQSVRRN |
| SEQ ID NO.: 399 | QSVRRN |
| SEQ ID NO.: 400 | SVRRN |
| SEQ ID NO.: 401 | VRRN |
| SEQ ID NO.: 402 | ATLSCRASQSVRR |
| SEQ ID NO.: 403 | TLSCRASQSVRR |
| SEQ ID NO.: 404 | LSCRASQSVRR |
| SEQ ID NO.: 405 | SCRASQSVRR |
| SEQ ID NO.: 406 | CRASQSVRR |
| SEQ ID NO.: 407 | RASQSVRR |
| SEQ ID NO.: 408 | ASQSVRR |
| SEQ ID NO.: 409 | SQSVRR |
| SEQ ID NO.: 410 | QSVRR |
| SEQ ID NO.: 411 | SVRR |
| SEQ ID NO.: 412 | ATLSCRASQSVR |
| SEQ ID NO.: 413 | TLSCRASQSVR |
| SEQ ID NO.: 414 | LSCRASQSVR |
| SEQ ID NO.: 415 | SCRASQSVR |
| SEQ ID NO.: 416 | CRASQSVR |
| SEQ ID NO.: 417 | RASQSVR |
| SEQ ID NO.: 418 | ASQSVR |
| SEQ ID NO.: 419 | SQSVR |
| SEQ ID NO.: 420 | QSVR |
| Light chain CDR2 region | |
| (SEQ ID NO: 421 to 513) | |
| SEQ ID NO.: 421 | RLLIYDASTRATGIPDR |
| SEQ ID NO.: 422 | LLIYDASTRATGIPDR |
| SEQ ID NO.: 423 | LIYDASTRATGIPDR |
| SEQ ID NO.: 424 | IYDASTRATGIPDR |
| SEQ ID NO.: 425 | YDASTRATGIPDR |
| SEQ ID NO.: 426 | DASTRATGIPDR |
| SEQ ID NO.: 427 | ASTRATGIPDR |
| SEQ ID NO.: 428 | STRATGIPDR |
| SEQ ID NO.: 429 | TRATGIPDR |
| SEQ ID NO.: 430 | RATGIPDR |
| SEQ ID NO.: 431 | ATGIPDR |
| SEQ ID NO.: 432 | RLLIYDASTRATGIPD |
| SEQ ID NO.: 433 | LLIYDASTRATGIPD |
| SEQ ID NO.: 434 | LIYDASTRATGIPD |
| SEQ ID NO.: 435 | IYDASTRATGIPD |
| SEQ ID NO.: 436 | YDASTRATGIPD |
| SEQ ID NO.: 437 | DASTRATGIPD |
| SEQ ID NO.: 438 | ASTRATGIPD |
| SEQ ID NO.: 439 | STRATGIPD |
| SEQ ID NO.: 440 | TRATGIPD |
| SEQ ID NO.: 441 | RATGIPD |
| SEQ ID NO.: 442 | ATGIPD |
| SEQ ID NO.: 443 | RLLIYDASTRATGIP |
| SEQ ID NO.: 444 | LLIYDASTRATGIP |
| SEQ ID NO.: 445 | LIYDASTRATGIP |
| SEQ ID NO.: 446 | IYDASTRATGIP |
| SEQ ID NO.: 447 | YDASTRATGIP |
| SEQ ID NO.: 448 | DASTRATGIP |
| SEQ ID NO.: 449 | ASTRATGIP |
| SEQ ID NO.: 450 | STRATGIP |
| SEQ ID NO.: 451 | TRATGIP |
| SEQ ID NO.: 452 | RATGIP |
| SEQ ID NO.: 453 | ATGIP |
| SEQ ID NO.: 454 | RLLIYDASTRATGI |
| SEQ ID NO.: 455 | LLIYDASTRATGI |
| SEQ ID NO.: 456 | LIYDASTRATGI |
| SEQ ID NO.: 457 | IYDASTRATGI |
| SEQ ID NO.: 458 | YDASTRATGI |
| SEQ ID NO.: 459 | DASTRATGI |
| SEQ ID NO.: 460 | ASTRATGI |
| SEQ ID NO.: 461 | STRATGI |
| SEQ ID NO.: 462 | TRATGI |
| SEQ ID NO.: 463 | RATGI |
| SEQ ID NO.: 464 | ATGI |
| SEQ ID NO.: 465 | RLLIYDASTRATG |
| SEQ ID NO.: 466 | LLIYDASTRATG |
| SEQ ID NO.: 467 | LIYDASTRATG |
| SEQ ID NO.: 468 | IYDASTRATG |
| SEQ ID NO.: 469 | YDASTRATG |
| SEQ ID NO.: 470 | DASTRATG |
| SEQ ID NO.: 471 | ASTRATG |
| SEQ ID NO.: 472 | STRATG |
| SEQ ID NO.: 473 | TRATG |
| SEQ ID NO.: 474 | RATG |
| SEQ ID NO.: 475 | RLLIYDASTRAT |
| SEQ ID NO.: 476 | LLIYDASTRAT |
| SEQ ID NO.: 477 | LIYDASTRAT |
| SEQ ID NO.: 478 | IYDASTRAT |
| SEQ ID NO.: 479 | YDASTRAT |
| SEQ ID NO.: 480 | DASTRAT |
| SEQ ID NO.: 481 | ASTRAT |
| SEQ ID NO.: 482 | STRAT |
| SEQ ID NO.: 483 | TRAT |
| SEQ ID NO.: 484 | RLLIYDASTRA |
| SEQ ID NO.: 485 | LLIYDASTRA |
| SEQ ID NO.: 486 | LIYDASTRA |
| SEQ ID NO.: 487 | IYDASTRA |
| SEQ ID NO.: 488 | YDASTRA |
| SEQ ID NO.: 489 | DASTRA |
| SEQ ID NO.: 490 | ASTRA |
| SEQ ID NO.: 491 | STRA |
| SEQ ID NO.: 492 | RLLIYDASTR |
| SEQ ID NO.: 493 | LLIYDASTR |
| SEQ ID NO.: 494 | LIYDASTR |
| SEQ ID NO.: 495 | IYDASTR |
| SEQ ID NO.: 496 | YDASTR |
| SEQ ID NO.: 497 | DASTR |
| SEQ ID NO.: 498 | ASTR |
| SEQ ID NO.: 499 | RLLIYDAST |
| SEQ ID NO.: 500 | LLIYDAST |
| SEQ ID NO.: 501 | LIYDAST |
| SEQ ID NO.: 502 | IYDAST |
| SEQ ID NO.: 503 | YDAST |
| SEQ ID NO.: 504 | DAST |
| SEQ ID NO.: 505 | RLLIYDAS |
| SEQ ID NO.: 506 | LLIYDAS |
| SEQ ID NO.: 507 | LIYDAS |
| SEQ ID NO.: 508 | IYDAS |
| SEQ ID NO.: 509 | YDAS |
| SEQ ID NO.: 510 | RLLIYDA |
| SEQ ID NO.: 511 | LLIYDA |
| SEQ ID NO.: 512 | LIYDA |
| SEQ ID NO.: 513 | IYDA |
| Light chain CDR3 region | |
| (SEQ ID NO: 514 to 628) | |
| SEQ ID NO.: 514 | AVYYCQQYDSSPPMYIFGQGT |
| SEQ ID NO.: 515 | VYYCQQYDSSPPMYIFGQGT |
| SEQ ID NO.: 516 | YYCQQYDSSPPMYIFGQGT |
| SEQ ID NO.: 517 | YCQQYDSSPPMYIFGQGT |
| SEQ ID NO.: 518 | CQQYDSSPPMYIFGQGT |
| SEQ ID NO.: 519 | QQYDSSPPMYIFGQGT |
| SEQ ID NO.: 520 | QYDSSPPMYIFGQGT |
| SEQ ID NO.: 521 | YDSSPPMYIFGQGT |
| SEQ ID NO.: 522 | DSSPPMYIFGQGT |
| SEQ ID NO.: 523 | SSPPMYIFGQGT |
| SEQ ID NO.: 524 | SPPMYIFGQGT |
| SEQ ID NO.: 525 | AVYYCQQYDSSPPMYIFGQG |
| SEQ ID NO.: 526 | VYYCQQYDSSPPMYIFGQG |
| SEQ ID NO.: 527 | YYCQQYDSSPPMYIFGQG |
| SEQ ID NO.: 528 | YCQQYDSSPPMYIFGQG |
| SEQ ID NO.: 529 | CQQYDSSPPMYIFGQG |
| SEQ ID NO.: 530 | QQYDSSPPMYIFGQG |
| SEQ ID NO.: 531 | QYDSSPPMYIFGQG |
| SEQ ID NO.: 532 | YDSSPPMYIFGQG |
| SEQ ID NO.: 533 | DSSPPMYIFGQG |
| SEQ ID NO.: 534 | SSPPMYIFGQG |
| SEQ ID NO.: 535 | SPPMYIFGQG |
| SEQ ID NO.: 536 | AVYYCQQYDSSPPMYIFGQ |
| SEQ ID NO.: 537 | VYYCQQYDSSPPMYIFGQ |
| SEQ ID NO.: 538 | YYCQQYDSSPPMYIFGQ |
| SEQ ID NO.: 539 | YCQQYDSSPPMYIFGQ |
| SEQ ID NO.: 540 | CQQYDSSPPMYIFGQ |
| SEQ ID NO.: 541 | QQYDSSPPMYIFGQ |
| SEQ ID NO.: 542 | QYDSSPPMYIFGQ |
| SEQ ID NO.: 543 | YDSSPPMYIFGQ |
| SEQ ID NO.: 544 | DSSPPMYIFGQ |
| SEQ ID NO.: 545 | SSPPMYIFGQ |
| SEQ ID NO.: 546 | SPPMYIFGQ |
| SEQ ID NO.: 547 | AVYYCQQYDSSPPMYIFG |
| SEQ ID NO.: 548 | VYYCQQYDSSPPMYIFG |
| SEQ ID NO.: 549 | YYCQQYDSSPPMYIFG |
| SEQ ID NO.: 550 | YCQQYDSSPPMYIFG |
| SEQ ID NO.: 551 | CQQYDSSPPMYIFG |
| SEQ ID NO.: 552 | QQYDSSPPMYIFG |
| SEQ ID NO.: 553 | QYDSSPPMYIFG |
| SEQ ID NO.: 554 | YDSSPPMYIFG |
| SEQ ID NO.: 555 | DSSPPMYIFG |
| SEQ ID NO.: 556 | SSPPMYIFG |
| SEQ ID NO.: 557 | SPPMYIFG |
| SEQ ID NO.: 558 | AVYYCQQYDSSPPMYIF |
| SEQ ID NO.: 559 | VYYCQQYDSSPPMYIF |
| SEQ ID NO.: 560 | YYCQQYDSSPPMYIF |
| SEQ ID NO.: 561 | YCQQYDSSPPMYIF |
| SEQ ID NO.: 562 | CQQYDSSPPMYIF |
| SEQ ID NO.: 563 | QQYDSSPPMYIF |
| SEQ ID NO.: 564 | QYDSSPPMYIF |
| SEQ ID NO.: 565 | YDSSPPMYIF |
| SEQ ID NO.: 566 | DSSPPMYIF |
| SEQ ID NO.: 567 | SSPPMYIF |
| SEQ ID NO.: 568 | SPPMYIF |
| SEQ ID NO.: 569 | AVYYCQQYDSSPPMYI |
| SEQ ID NO.: 570 | VYYCQQYDSSPPMYI |
| SEQ ID NO.: 571 | YYCQQYDSSPPMYI |
| SEQ ID NO.: 572 | YCQQYDSSPPMYI |
| SEQ ID NO.: 573 | CQQYDSSPPMYI |
| SEQ ID NO.: 574 | QQYDSSPPMYI |
| SEQ ID NO.: 575 | QYDSSPPMYI |
| SEQ ID NO.: 576 | YDSSPPMYI |
| SEQ ID NO.: 577 | DSSPPMYI |
| SEQ ID NO.: 578 | SSPPMYI |
| SEQ ID NO.: 579 | SPPMYI |
| SEQ ID NO.: 580 | AVYYCQQYDSSPPMY |
| SEQ ID NO.: 581 | VYYCQQYDSSPPMY |
| SEQ ID NO.: 582 | YYCQQYDSSPPMY |
| SEQ ID NO.: 583 | YCQQYDSSPPMY |
| SEQ ID NO.: 584 | CQQYDSSPPMY |
| SEQ ID NO.: 585 | QQYDSSPPMY |
| SEQ ID NO.: 586 | QYDSSPPMY |
| SEQ ID NO.: 587 | YDSSPPMY |
| SEQ ID NO.: 588 | DSSPPMY |
| SEQ ID NO.: 589 | SSPPMY |
| SEQ ID NO.: 590 | SPPMY |
| SEQ ID NO.: 591 | AVYYCQQYDSSPPM |
| SEQ ID NO.: 592 | VYYCQQYDSSPPM |
| SEQ ID NO.: 593 | YYCQQYDSSPPM |
| SEQ ID NO.: 594 | YCQQYDSSPPM |
| SEQ ID NO.: 595 | CQQYDSSPPM |
| SEQ ID NO.: 596 | QQYDSSPPM |
| SEQ ID NO.: 597 | QYDSSPPM |
| SEQ ID NO.: 598 | YDSSPPM |
| SEQ ID NO.: 599 | DSSPPM |
| SEQ ID NO.: 600 | SSPPM |
| SEQ ID NO.: 601 | SPPM |
| SEQ ID NO.: 602 | AVYYCQQYDSSPP |
| SEQ ID NO.: 603 | VYYCQQYDSSPP |
| SEQ ID NO.: 604 | YYCQQYDSSPP |
| SEQ ID NO.: 605 | YCQQYDSSPP |
| SEQ ID NO.: 606 | CQQYDSSPP |
| SEQ ID NO.: 607 | QQYDSSPP |
| SEQ ID NO.: 608 | QYDSSPP |
| SEQ ID NO.: 609 | YDSSPP |
| SEQ ID NO.: 610 | DSSPP |
| SEQ ID NO.: 611 | SSPP |
| SEQ ID NO.: 612 | AVYYCQQYDSSP |
| SEQ ID NO.: 613 | VYYCQQYDSSP |
| SEQ ID NO.: 614 | YYCQQYDSSP |
| SEQ ID NO.: 615 | YCQQYDSSP |
| SEQ ID NO.: 616 | CQQYDSSP |
| SEQ ID NO.: 617 | QQYDSSP |
| SEQ ID NO.: 618 | QYDSSP |
| SEQ ID NO.: 619 | YDSSP |
| SEQ ID NO.: 620 | DSSP |
| SEQ ID NO.: 621 | AVYYCQQYDSS |
| SEQ ID NO.: 622 | VYYCQQYDSS |
| SEQ ID NO.: 623 | YYCQQYDSS |
| SEQ ID NO.: 624 | YCQQYDSS |
| SEQ ID NO.: 625 | CQQYDSS |
| SEQ ID NO.: 626 | QQYDSS |
| SEQ ID NO.: 627 | QYDSS |
| SEQ ID NO.: 628 | YDSS |
| Heavy chain variable domain | |
| (SEQ ID NO: 629) | |
| QEQLVQSGAEVKKPGASVKVSCKSSGFTFSYFYLHWVRQAPGQGLEWMGIINPRGD | |
| GTRYAQKFQGRVTMTRDASTGTLYMELRSLRSEDTAVYYCARGADHGAFDIWGQGT | |
| MVTVSS | |
| Light chain variable domain | |
| (SEQ ID NO: 630) | |
| EIVLTQSPGTLSLSPGERATLSCRASQSVRRNYFAWYQQKRGQAPRLLIYDASTRATG | |
| IPDRFSGSGSGTDFTLTISRLEPEDFAVYYCQQYDSSPPMYIFGQGTKLEIK | |
| Heavy chain FR1 region | |
| (SEQ ID NO: 631) | |
| SEQ ID NO: 631 | QEQLVQSGAEVKKPGASVKVSCKSSGFTFS |
| Heavy chain FR2 region | |
| (SEQ ID NO: 632) | |
| SEQ ID NO: 632 | WVRQAPGQGLEWMG |
| Heavy chain FR3 region | |
| (SEQ ID NO: 633) | |
| SEQ ID NO: 633 | RVTMTRDASTGTLYMELRSLRSEDTAVYYCAR |
| Heavy chain FR4 region | |
| (SEQ ID NO: 634) | |
| SEQ ID NO: 634 | WGQGTMVTVSS |
| Light chain FR1 region | |
| (SEQ ID NO: 635) | |
| SEQ ID NO: 635 | EIVLTQSPGTLSLSPGERATLSC |
| Light chain FR2 region | |
| (SEQ ID NO: 636) | |
| SEQ ID NO: 636 | WYQQKRGQAPRLLIY |
| Light chain FR3 region | |
| (SEQ ID NO: 637) | |
| SEQ ID NO: 637 | GIPDRFSGSGSGTDFTLTISRLEPEDFAVYYC |
| Light chain FR4 region | |
| (SEQ ID NO: 638) | |
| SEQ ID NO: 638 | FGQGTKLEIK |
| Heavy chain CDR1 region | |
| (SEQ ID NO: 639) | |
| SEQ ID NO.: 639 | DYRIH |
| Heavy chain CDR2 region | |
| (SEQ ID NO: 640) | |
| SEQ ID NO.: 640 | RMNPKSGDTNFAQKFQG |
| Heavy chain CDR3 region | |
| (SEQ ID NO: 641) | |
| SEQ ID NO.: 641 | LLIVGGFDPLDDFEV |
| Light chain CDR1 region | |
| (SEQ ID NO: 642) | |
| SEQ ID NO.: 642 | SGTSSDVGGYNFVS |
| Light chain CDR2 region | |
| (SEQ ID NO: 643) | |
| SEQ ID NO.: 643 | EVTKRPS |
| Light chain CDR3 region | |
| (SEQ ID NO: 644) | |
| SEQ ID NO.: 644 | SSYGGTNNLL |
| Heavy chain CDR1 region | |
| (SEQ ID NO: 645) | |
| SEQ ID NO.: 645 | GYAMH |
| Heavy chain CDR2 region | |
| (SEQ ID NO: 646) | |
| SEQ ID NO.: 646 | VISRDARNKYYADSVKG |
| Heavy chain CDR3 region | |
| (SEQ ID NO: 647) | |
| SEQ ID NO.: 647 | LIIPGITEPGSPDALDI |
| Light chain CDR1 region | |
| (SEQ ID NO: 648) | |
| SEQ ID NO.: 648 | RASQDISKWLA |
| Light chain CDR2 region | |
| (SEQ ID NO: 649) | |
| SEQ ID NO.: 649 | AASSLQS |
| Light chain CDR3 region | |
| (SEQ ID NO: 650) | |
| SEQ ID NO.: 650 | QQASSFPWSIT |
| Heavy chain CDR1 region | |
| (SEQ ID NO: 651) | |
| SEQ ID NO.: 651 | SHYMH |
| Heavy chain CDR2 region | |
| (SEQ ID NO: 652) | |
| SEQ ID NO.: 652 | IINPSGSGTAYGQKFQG |
| Heavy chain CDR3 region | |
| (SEQ ID NO: 653) | |
| SEQ ID NO.: 653 | GSGGLFAY |
| Light chain CDR1 region | |
| (SEQ ID NO: 654) | |
| SEQ ID NO.: 654 | RASQIVRSNYLA |
| Light chain CDR2 region | |
| (SEQ ID NO: 655) | |
| SEQ ID NO.: 655 | GASSRAT |
| Light chain CDR3 region | |
| (SEQ ID NO: 656) | |
| SEQ ID NO.: 656 | LQYDSSPPTYI |
| Heavy chain CDR1 region | |
| (SEQ ID NO: 657) | |
| SEQ ID NO.: 657 | SYYMH |
| Heavy chain CDR2 region | |
| (SEQ ID NO: 658) | |
| SEQ ID NO.: 658 | LITPSGDDTYYAQRFQG |
| Heavy chain CDR3 region | |
| (SEQ ID NO: 659) | |
| SEQ ID NO.: 659 | MSRAGGFDV |
| Light chain CDR1 region | |
| (SEQ ID NO: 660) | |
| SEQ ID NO.: 660 | RASQSITGRYLA |
| Light chain CDR2 region | |
| (SEQ ID NO: 661) | |
| SEQ ID NO.: 661 | GESSRVT |
| Light chain CDR3 region | |
| (SEQ ID NO: 662) | |
| SEQ ID NO.: 662 | QHFASSPPTYT |
| Heavy chain arm | |
| (SEQ ID NO: 663) | |
| SEQ ID NO.: 663 | QEQLVQSGAEVKKPGASVKVSCKSSGFTFSYFYLHWVRQAPGQGLEWMGIINPRGD |
| | GTRYAQKFQGRVTMTRDASTGTLYMELRSLRSEDTAVYYCARGADHGAFDIWGQGT |
| | MVTVSSASVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFNRGECD |
| | KTHTCPPCPAPELLGGPSVFLFPPKPKDTLMISRTPEVTCVVVDVSHEDPEVKENWY |
| | VDGVEVHNAKTKPREEQYNSTYRVVSVLTVLHQDWLNGKEYKCKVSNKALPAPIEK |
| | TISKAKGQPREPQVCTLPPSRDELTKNQVSLSCAVKGFYPSDIAVEWESNGQPENNY |
| | KTTPPVLDSDGSFFLVSKLTVDKSRWQQGNVFSCSVMHEALHNHYTQKSLSLSPGK |
| Light chain arm | |
| (SEQ ID NO: 664) | |
| SEQ ID NO.: 664 | EIVLTQSPGTLSLSPGERATLSCRASQSVRRNYFAWYQQKRGQAPRLLIYDASTRATG |
| | IPDRFSGSGSGTDFTLTISRLEPEDFAVYYCQQYDSSPPMYIFGQGTKLEIKSSASTKG |
| | PSVFPLAPSSKSTSGGTAALGCLVKDYFPEPVTVSWNSGALTSGVHTFPAVLQSSGLY |
| | SLSSVVTVPSSSLGTQTYICNVNHKPSNTKVDKKVEPKSC |
| Heavy chain arm | |
| (SEQ ID NO: 665) | |
| SEQ ID NO.: 665 | QMQLMQSGAEVKKPGASVTVSCKASGDTFSDYRIHWVRQAPGQGLEWMGRMNPKS |
| | GDTNFAQKFQGRVTMTRDMSINTAYMTLSGLTFDDTALYYCASLLIVGGFDPLDDFE |
| | VWGQGTMVTISSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVSHEDPEVKENW |
| | YVDGVEVHNAKTKPREEQYNSTYRVVSVLTVLHQDWLNGKEYKCKVSNKALPAPIE |
| | KTISKAKGQPREPQVYTLPPCRDELTKNQVSLWCLVKGFYPSDIAVEWESNGQPENN |
| | YKTTPPVLDSDGSFFLYSKLTVDKSRWQQGNVFSCSVMHEALHNHYTQKSLSLSPG |
| | K |
| Light chain arm | |
| (SEQ ID NO: 666) | |
| SEQ ID NO.: 666 | QSALTQPPSASGSPGQSVTISCSGTSSDVGGYNFVSWYQHHPGKAPKILIYEVTKRPS |
| | GVPDRFSGSKSGNTASLTVSGLQAEDEADYYCSSYGGTNNLLFGGGTKLTVLGQPKA |
| | APSVTLFPPSSEELQANKATLVCLISDFYPGAVTVAWKADSSPVKAGVETTTPSKQSN |
| | NKYAASSYLSLTPEQWKSHRSYSCQVTHEGSTVEKTVAPTECS |
| Heavy chain arm | |
| (SEQ ID NO: 667) | |
| SEQ ID NO.: 667 | QEQLVQSGAEVKKPGASVKVSCKSSGFTFSYFYLHWVRQAPGQGLEWMGIINPRGD |
| | GTRYAQKFQGRVTMTRDASTGTLYMELRSLRSEDTAVYYCARGADHGAFDIWGQGT |
| | MVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKA |
| | KGQPREPQVYTLPPCRDELTKNQVSLWCLVKGFYPSDIAVEWESNGQPENNYKTTP |
| | PVLDSDGSFFLYSKLTVDKSRWQQGNVFSCSVMHEALHNHYTQKSLSLSPGK |
| Light chain arm | |
| (SEQ ID NO: 668) | |
| SEQ ID NO.: 668 | EIVLTQSPGTLSLSPGERATLSCRASQSVRRNYFAWYQQKRGQAPRLLIYDASTRATG |
| | IPDRFSGSGSGTDFTLTISRLEPEDFAVYYCQQYDSSPPMYIFGQGTKLEIKRTVAAPS |
| | VFIFPPSDEQLKSGTASVVCLLNNFYPREAKVQWKVDNALQSGNSQESVTEQDSKDS |
| | TYSLSSTLTLSKADYEKHKVYACEVTHQGLSSPVTKSFNRGEC |
| Heavy chain arm | |
| (SEQ ID NO: 669) | |
| SEQ ID NO.: 669 | QMQLMQSGAEVKKPGASVTVSCKASGDTFSDYRIHWVRQAPGQGLEWMGRMNPKS |
| | GDTNFAQKFQGRVTMTRDMSINTAYMTLSGLTFDDTALYYCASLLIVGGFDPLDDFE |
| | VWGQGTMVTISGQPKAAPSVTLFPPSSEELQANKATLVCLISDFYPGAVTVAWKADS |
| | SPVKAGVETTTPSKQSNNKYAASSYLSLTPEQWKSHRSYSCQVTHEGSTVEKTVAPT |
| | ECSDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVCTLPPSRDELTKNQVSLSCAVKGFYPSDIAVEWESNGQPE |
| | NNYKTTPPVLDSDGSFFLVSKLTVDKSRWQQGNVFSCSVMHEALHNHYTQKSLSLS |
| | PGK |
| Light chain arm | |
| (SEQ ID NO: 670) | |
| SEQ ID NO.: 670 | QSALTQPPSASGSPGQSVTISCSGTSSDVGGYNFVSWYQHHPGKAPKILIYEVTKRPS |
| | GVPDRFSGSKSGNTASLTVSGLQAEDEADYYCSSYGGTNNLLFGGGTKLTVLSSAST |
| | KGPSVFPLAPSSKSTSGGTAALGCLVKDYFPEPVTVSWNSGALTSGVHTFPAVLQSSG |
| | LYSLSSVVTVPSSSLGTQTYICNVNHKPSNTKVDKKVEPKSC |
| Heavy chain arm | |
| (SEQ ID NO: 671) | |
| SEQ ID NO.: 671 | QSALTQPPSASGSPGQSVTISCSGTSSDVGGYNFVSWYQHHPGKAPKILIYEVTKRPS |
| | GVPDRFSGSKSGNTASLTVSGLQAEDEADYYCSSYGGTNNLLFGGGTKLTVLGQPKA |
| | APSVTLFPPSSEELQANKATLVCLISDFYPGAVTVAWKADSSPVKAGVETTTPSKQSN |
| | NKYAASSYLSLTPEQWKSHRSYSCQVTHEGSTVEKTVAPTECSDKTHTCPPCPAPEL |
| | LGGPSVFLFPPKPKDTLMISRTPEVTCVVVDVSHEDPEVKFNWYVDGVEVHNAKTK |
| | PREEQYNSTYRVVSVLTVLHQDWLNGKEYKCKVSNKALPAPIEKTISKAKGQPREPQ |
| | VCTLPPSRDELTKNQVSLSCAVKGFYPSDIAVEWESNGQPENNYKTTPPVLDSDGSF |
| | FLVSKLTVDKSRWQQGNVFSCSVMHEALHNHYTQKSLSLSPGK |
| Light chain of arm | |
| (SEQ ID NO: 672) | |
| SEQ ID NO.: 672 | QMQLMQSGAEVKKPGASVTVSCKASGDTFSDYRIHWVRQAPGQGLEWMGRMNPKS |
| | GDTNFAQKFQGRVTMTRDMSINTAYMTLSGLTFDDTALYYCASLLIVGGFDPLDDFE |
| | VWGQGTMVTISSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKVEPKSC |

Heavy chain variable domain
(SEQ ID NO: 673)
QMQLMQSGAEVKKPGASVTVSCKASGDTFSDYRIHWVRQAPGQGLEWMGRMNPKS
GDTNFAQKFQGRVTMTRDMSINTAYMTLSGLTFDDTALYYCASLLIVGGFDPLDDFE
VWGQGTMVTISS
Light chain variable domain
(SEQ ID NO: 674)
QSALTQPPSASGSPGQSVTISCSGTSSDVGGYNFVSWYQHHPGKAPKILIYEVTKRPS
GVPDRFSGSKSGNTASLTVSGLQAEDEADYYCSSYGGTNNLLFGGGTKLTVL
Heavy chain variable domain
(SEQ ID NO: 675)
QVQLVESGGGVVQPGRSLRLSCAASGLTFSGYAMHWVRQAPGKGLEWVAVISRDAR
NKYYADSVKGRFTISRDNSKKTVYLEMNSLRVEDTAVYYCAILIIPGITEPGSPDALDI
WGQGTMVSVSS
Light chain variable domain
(SEQ ID NO: 676)
DIQMTQSPSSMSASVGDRVTITCRASQDISKWLAWYQQRPGKAPKLLIYAASSLQSGV
PSRFSGSGSGTDFTLTISSLQPEDFATYYCQQASSFPWSITFGQGTRLEIR

### EXAMPLES

The monoclonal antibody CV3-25 has shown in vitro protection against the original Wuhan strain of SARS-COV-2 binding specifically to the stem helix of SARS-COV-2, a binding region shared with antibodies of the present invention. When administered mucosally at lower dosages, CV3-25 affords a lower level of protection when compared to its systemic administration. In contrast, however, when antibodies of the present invention are administered mucosally they afford a greater level of protection as demonstrated by the in vivo studies presented below.

### Example 1: CV3-25

The aim of this study was to assess the pre-exposure efficacy of the monoclonal antibody CV3-25 following systemic intraperitoneal administration in a SARS-COV-2 Delta K18 hACE2 Tg mouse model.

Animals (n=10 per group) were treated with an intraperitoneal dose range of the test CV3-25 (15 to 0.2 mg/kg) or PBS control as vehicle at study day −1. At study day 0, animals were challenged with 10^{3.5} TCID₅₀ of SARS-COV-2 Delta variant.

### Materials and Methods

The monoclonal antibody CV3-25 was dissolved and diluted with phosphate-buffered saline (PBS), thereby preparing a CV3-25 dilution.

The CV3-25 dilution was made up at various concentrations so that 200 μL administrations to the mean weight of the dosing group resulted in CV3-25 doses ranging from 15 mg/kg, 5 mg/kg, 1.7 mg/kg, 0.5 mg/kg to 0.2 mg/kg.

The antibody was stored at −80° C.±10° C. and dilutions were stored at 4° C. until usage and the temperature of the storage unit was monitored.

The virus strain tested was SARS-COV-2 Delta variant (Aus/VIC/18440/2021 (B.1.617.2), which has been passed three times through Vero-hSLAM cells.

### Animals

The animal species used was the SARS-COV-2 Delta K18 hACE2 Tg (B6.Cg-Tg (K18-ACE2) 2Prlmn/JArc; Jax™ Stock No.: 034860) mouse model, at a weight of c. 17-26 g on day −9. 60 mice were used at the age of 11-13 weeks on the day of test article administration and were 83.3% female. Ten animals were randomly allocated to 6 treatment groups based on day −9 weights to create groups with comparable mean weight, with 20% males per group, except 15 mg/kg which was exclusively female. All mice were housed in individually ventilated cages (IVCs), with corn cob bedding, tissues or shredded paper as nesting material, a wooden chew block, accessible food pellets and acidic water for animal nourishment and red plastic tunnels. Each cage system holds 3 to 5 mice per cage. All mice were transferred to fresh cages every 14 days. Food and water were inspected daily and topped up as needed.

### Study Design

The dose level of CV3-25 applied in the current example was based on a dose range proven to have prophylactic activity. A total number of 60 mice, 11-13 weeks of age were transported to the animal facility and allocated to 6 experimental groups according to Table 1 (see below). Mice were given a period of >3 days for acclimatization.

TABLE 1
Intraperitoneal experimental detail

| Test | | Admin. | Dose | Day of | No. of |
|---|---|---|---|---|---|
| group | Antibody | route | (mg/kg) | admin. | Animals |
| 15 | CV3-25 | I.P. | 15 | −1 | 10 |
| 5 | CV3-25 | I.P. | 5 | −1 | 10 |
| 1.7 | CV3-25 | I.P. | 1.7 | −1 | 10 |
| 0.5 | CV3-25 | I.P. | 0.5 | −1 | 10 |
| 0.2 | CV3-25 | I.P. | 0.2 | −1 | 10 |
| Vehicle | — | I.P. | 0 | −1 | 10 |

SARS-COV-2 Delta K18 hACE2 mice (20% males per group, except 15 mg/kg which was exclusively female) were treated via the Intraperitoneal route of administration with the antibody at a dose between 15 mg/kg and 0.2 mg/kg based upon the average group weight at day −9 adjusted with a 4% average weight gain (based on a subset of the animals weighted at day −1). On Day 0, all mice were challenged with a lethal dose (10^{3.5} TCID₅₀) of the SARS-COV-2 Delta variant and observed for weight loss and mortality until the end of the study at day 11.

### Antibody Administration

The test antibody was stored at −80° C.±10° C. upon arrival. The appropriate dose, according to the treatment schedule (Table1), was formulated according to average group weight at day −9 adjusted with a 4% average weight gain (based on a subset of the animals weighted at day −1).

Prior to dosing, the material was drawn into a 1 mL syringe with a 26G needle, allowed briefly to warm to room temperature and then administered to each mouse. Mice receive the indicated dose by intraperitoneal administration of 200 μL of antibody solution into the intraperitoneal cavity, mice were scruffed and held inverted at a −45 deg angle for administration (200 μL per mouse).

### Virus Administration

The virus material was stored at −80° C.±10° C. and was defrosted prior to administration. Once defrosted, the material was diluted in sterile PBS corresponding to approximately 10^{3.5} TCID₅₀/50 μL. As required, the animals were anesthetized by isoflurane (4% v/v with 2 L/min (2) and each animal received approximately 50 μL (25 μl in each nare) of virus corresponding with approximately 10^{3.5} TCID₅₀ by intranasal inoculation. Unused material was frozen at −20° C. to −80° C. for back titration.

### Laboratory Analysis

Inoculum was back titrated and the dose of the virus administered was verified by titrating replicate samples on Vero cells.

### Clinical Monitoring

General health observations were performed on each animal from the day of arrival until the end of the study at least once daily (during normal servicing procedures). Each animal was weighed daily, beginning at the day of infection (day 0).

### Terminal Investigations

At the end of the study, on day 11, mice were euthanized by cervical dislocation. Gross necropsy was not performed.

### Data Analysis and Statistical Methods

Survival proportions at day 11, survival times and change in bodyweight (Area Under the Curve) were compared to the control group using Fisher's exact test, log-rank and Welch's t-test, respectively. All groups were compared to the vehicle (PBS solution) control group. P-values were adjusted according to Bonferroni (for two comparisons to vehicle) followed by a stepwise approach (starting with the highest antibody dose conditionally testing a lower dose if the previous step was statistically significant).

Statistical analysis was performed using R and statistical significance was set at α=0.05.

### Survival

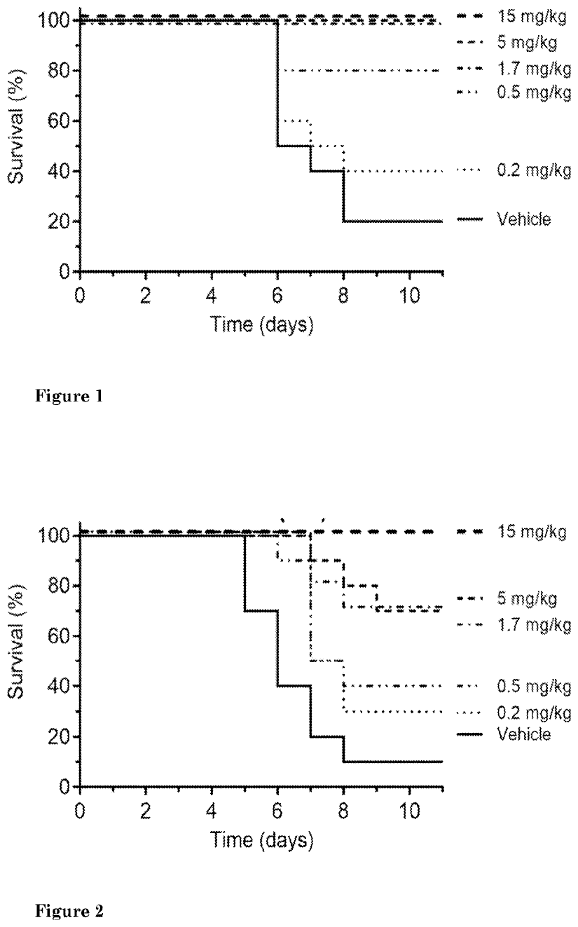

Prophylactic treatment with ≥0.5 mg/kg test antibody provided a statistically significant increase in survival compared to the control group (See FIG. 1). The animals that were treated with 0.5 mg/kg test antibody had a 80% survival rate, animals treated with ≥1.7 mg/kg had a survival rate of 100%, whereas the survival proportion at day 11 in the control group was 20%. The median survival time of the control group was 6.5 days.

Prophylactic treatment with ≥0.5 mg/kg test antibody resulted in a significant improvement in survival time compared to the control group.

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline and the net AUC was defined as the summation of the area above and below the baseline using the percentage change per day. Prophylactic treatment with ≥0.5 mg/kg test antibody resulted in a significant reduction in weight loss compared to the control group (see FIG. 8).

### CONCLUSION

In this lethal SARS-COV-2 Delta K18 hACE2 Tg mouse model, the prophylactic intraperitoneal administration of ≥0.5 mg/kg test antibody provides a statistically significant improvement in survival and a reduction in weight loss compared to control, whereas eight out of ten animals in the control group did not survive.

### Example 2: CV3-25

The aim of this study was to assess the pre-exposure efficacy of the monoclonal antibody CV3-25 following intranasal administration in a SARS-COV-2 Delta K18 hACE2 Tg mouse model.

Animals (n=10 per group) were treated with an intranasal dose range of the test CV3-25 (15 to 0.2 mg/kg) or PBS control as vehicle on study day −1. At study day 0, animals were challenged with 10^{3.5} TCID₅₀ of SARS-COV-2 Delta variant.

### Materials and Methods

The monoclonal antibody CV3-25 was dissolved and diluted with phosphate-buffered saline (PBS), thereby preparing a CV3-25 dilution.

The CV3-25-dilution was made up at various concentrations so that 50 μL administrations to the mean weight of the dosing group resulted in CV3-25 doses ranging from 15 mg/kg, 5 mg/kg, 1.7 mg/kg, 0.5 mg/kg to 0.2 mg/kg.

The formulations were stored at −80° C.±10° C. and the temperature of the storage unit was monitored.

The virus strain tested was SARS-COV-2 Delta variant (Aus/VIC/18440/2021 (B.1.617.2), which had been passed three times through Vero-hSLAM cells.

### Animals

The animal species used was the SARS-COV-2 Delta K18 hACE2 Tg (B6.Cg-Tg (K18-ACE2) 2Prlmn/JArc; Jax™ Stock No.: 034860) mouse model, at a weight of c. 17-26 g on day −9. 60 mice were used at the age of 11-13 weeks on the day of test article administration and were 83.3% female. Ten animals were randomly allocated to 6 treatment groups, based on day −9 weights to create groups with comparable mean weight, with 20% males per group, except 15 mg/kg which was exclusively female. All mice were housed in individually ventilated cages (IVCs), with corn cob bedding, tissues or shredded paper as nesting material, a wooden chew block, accessible food pellets and acidic water for animal nourishment and red plastic tunnels. Each cage system holds 3 to 5 mice per cage. All mice were transferred to fresh cages every 14 days. Food and water were inspected daily and topped up as needed.

### Study Design

The dose level of CV3-25 applied in the current example was extrapolated from a dose range proven to have prophylactic activity intravenously. A total number of 60 mice, 11-13 weeks of age at the time of arrival, were transported to the animal facility and allocated to 6 experimental groups according to Table 2 (see below). Mice were given a period of >3 days for acclimatization.

TABLE 2
Intranasal experimental detail

| Test | | Admin. | Dose | Day of | No. of |
|---|---|---|---|---|---|
| group | Antibody | route | (mg/kg) | admin. | Animals |
| 15 | CV3-25 | I.N. | 15 | −1 | 10 |
| 5 | CV3-25 | I.N. | 5 | −1 | 10 |
| 1.7 | CV3-25 | I.N. | 1.7 | −1 | 10 |
| 0.5 | CV3-25 | I.N. | 0.5 | −1 | 10 |
| 0.2 | CV3-25 | I.N. | 0.2 | −1 | 10 |
| Vehicle | — | I.N. | 0 | −1 | 10 |

Female SARS-COV-2 Delta K18 hACE2 mice (20% males per group, except 15 mg/kg which was exclusively female) were treated via the intranasal route of administration with the antibody at a dose between 15 mg/kg and 0.2 mg/kg based upon the average weight at day −9 adjusted with a 4% average weight gain (based on a subset of the animals weighted at day −1). On Day 0, all mice were challenged with a lethal dose (10^{3.5} TCID₅₀) of the SARS-COV-2 Delta variant and observed for weight loss and mortality until the end of the study at day 11.

### Antibody Administration

The test antibody was stored at −80° C.±10° C. upon arrival. The appropriate dose, according to the treatment schedule (Table 2), was formulated according to average group weight at day −9 adjusted with a 4% average weight gain (based on a subset of animals weighted at day −1).

Just prior to dosing, the material was drawn into a syringe, allowed briefly to warm to room temperature and then administered to each mouse. Mice were anaesthetized with isoflurane (4% v/v with 2 L/min O₂) for two minutes. The mice were then scruffed and held at a +45 deg angle. 50 μL of compound or vehicle control was then slowly administered to the nares of each mouse (25 μL each nare). The mice were held for an additional 5 to 10 seconds to ensure compound delivery to the lower respiratory tract before being put back into the home box.

### Virus Administration

The virus material was stored at −80° C.±10° C. and was defrosted prior to administration. Once defrosted, the material was diluted in cold PBS corresponding to approximately 10^{3.5} TCID₅₀/50 μL. As required, the animals were anesthetized with isoflurane (4% v/v with 2 L/min O₂) and each animal received approximately 50 μL (25 μL each nare) of virus corresponding with approximately 10^{3.5} TCID₅₀ by intranasal inoculation using a pipette tip. Unused material was frozen at −20° C. to −80° C. for back titration.

### Laboratory Analysis

Inoculum was returned to the lab and the dose of the virus administered was verified by titrating replicate samples on Vero cells.

### Clinical Monitoring

General health observations were performed on each animal from the day of arrival until the end of the study at least once daily (during normal servicing procedures). Each animal was weighed daily beginning one day prior to infection (day −1).

### Terminal Investigations

At the end of the study, on day 11, mice were euthanized by cervical dislocation. Gross necropsy was not performed

### Data Analysis and Statistical Methods

Two animals in the 15 mg/kg CV3-25 were misdosed (sneezed out part of the antibody volume). These two animals were excluded from the statistical analysis.

Survival proportions at day 11, survival times and change in bodyweight (Area Under the Curve) were compared to the control group using Fisher's exact test, log-rank and Welch's t-test, respectively. All groups were compared to the vehicle (PBS solution) control group. P-values were adjusted according to Bonferroni (for two comparisons to vehicle) followed by a stepwise approach (starting with the highest antibody dose conditionally testing a lower dose if the previous step was statistically significant).

Statistical analysis was performed using R and statistical significance was set at α=0.05.

### Survival

Prophylactic treatment with ≥1.7 mg/kg test antibody provided statistically significant increase in survival compared to the control group (See FIG. 2). The median survival time of the control group was 6.0 days.

Prophylactic treatment with ≥1.7 mg/kg test antibody resulted in a significant improvement in survival time compared to the control group.

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline and the net AUC was defined as the summation of the area above and below the baseline using the percentage change per day. Prophylactic treatment with ≥0.2 mg/kg test antibody resulted in a significant reduction in weight loss compared to the control group (see FIG. 9).

### CONCLUSION

In this lethal SARS-COV-2 Delta K18 hACE2 Tg mouse model, the prophylactic intranasal administration of ≥1.7 mg/kg test antibody provides a statistically significant improvement in survival and a reduction in weight loss compared to control. In contrast, the control group showed 10% survival with a median survival time of 6 days.

### Example 3: Antibody According to the Invention

The aim of this study was to assess the pre-exposure efficacy of a monoclonal antibody according to the invention, having a heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574, following systemic intraperitoneal administration in a SARS-COV-2 Delta K18 hACE2 Tg mouse model.

Animals (n=10 per group) were treated with an intraperitoneal dose range of the test antibody described above (10 to 0.2 mg/kg) or PBS control as vehicle at study day −1. At study day 0, animals were challenged with 10^{3.5} TCID₅₀ of SARS-COV-2 Delta variant.

### Materials and Methods

The monoclonal test antibody described above, was buffered in phosphate-buffered saline PBS was diluted to the final concentrations for administration (10 to 0.2 mg/kg in 200 μL). thereby preparing the test antibody dilution.

The test antibody dilution was made up at various concentrations so that 200 μL administrations to the mean weight per cage of the dosing group resulted in test antibody doses ranging from 10 mg/kg, 5 mg/kg, 1.7 mg/kg, 0.5 mg/kg to 0.2 mg/kg.

The antibody was stored at −80° C.±10° C. and dilutions were stored at 4° C. until usage and the temperature of the storage unit was monitored.

The virus strain tested was SARS-COV-2 Delta variant (Aus/VIC/18440/2021 (B.1.617.2), which had been passed three times through Vero-hSLAM cells.

### Animals

The animal species used was the SARS-COV-2 Delta K18 hACE2 Tg (B6.Cg-Tg (K18-ACE2) 2Prlmn/JArc; Jax™ Stock No.: 034860) mouse model, at a weight of c. 17-22 g at day −5. 60 mice were used at the age of 7-11 weeks and were exclusively female. Ten animals were allocated to 6 treatment groups based on day −5 weight to create groups with similar mean weight. All mice were housed in individually ventilated cages (IVCs), with corn cob bedding, tissues or shredded paper as nesting material, a wooden chew block, accessible food pellets and acidic water for animal nourishment and red plastic tunnels. Each cage system holds 3 to 5 mice per cage. All mice were transferred to fresh cages every 14 days. Food and water were inspected daily and topped up as needed. Each animal was weighed daily beginning at the day of infection (day 0).

### Study Design

The dose level of test antibody applied in the current example was based on the maximal dosing possible for the material stock concentration (4 mg/mL) and based on literature and previous experience with an anti-S2 antibody (CV3-25) showing protection IP at doses ≥0.5 mg/kg. A total number of 60 mice, 7-11 weeks of age, were allocated to 6 experimental groups according to Table 3 (see below). Mice were given a period of >3 days for acclimatization.

TABLE 3
Intraperitoneal experimental detail

| Test | | Admin. | Dose | Day of | No. of |
|---|---|---|---|---|---|
| group | Antibody | route | (mg/kg) | admin. | Animals |
| 10 | As | I.P. | 10 | −1 | 10 |
| 5 | disclosed | I.P. | 5 | −1 | 10 |
| 1.7 | above. | I.P. | 1.7 | −1 | 10 |
| 0.5 | | I.P. | 0.5 | −1 | 10 |
| 0.2 | | I.P. | 0.2 | −1 | 10 |
| Vehicle | — | I.N. | 0 | −1 | 10 |

Female SARS-COV-2 Delta K18 hACE2 mice were treated via the intraperitoneal route of administration with the antibody at a dose between 10 mg/kg and 0.2 mg/kg based upon average group weight at day −5. On Day 0, all mice were challenged with a lethal dose (10^{3.5} TCID₅₀) of the SARS-COV-2 Delta variant and observed for weight loss and mortality until the end of the study at day 14.

### Antibody Administration

The test antibody was stored at −80° C.±10° C. upon arrival. The appropriate dose, according to the treatment schedule (Table 3), was formulated using the average weight per cage.

Prior to dosing, the material was drawn into a 1 mL syringe with a 26G needle and then administered to each mouse. Mice in the treatment group received the indicated dose by intraperitoneal administration of 200 μL of antibody solution into the intraperitoneal cavity, mice were scruffed and held inverted at a −45 deg angle for administration (200 μL per mouse). Mice in the vehicle group that received PBS were anaesthetized with isoflurane (4% v/v with 2 L/min O₂) for two minutes. The mice were then scruffed and held at a +45 deg angle. 50 μL of compound or vehicle control was then slowly administered to the nares of each mouse (25 μL each nare). The mice were held for an additional 5 to 10 seconds to ensure compound delivery to the lower respiratory tract before being put back into the home box.

### Virus Administration

The virus material was stored at −80° C.±10° C. and was defrosted prior to administration, at a titer of 10^{6.3} TCID₅₀/mL. Once defrosted, the material was diluted in sterile PBS corresponding to approximately 10^{3.5} TCID₅₀/50 μL). As required, the animals were anesthetized by isoflurane (4% v/v with 2 L/min O₂) and each animal received approximately 50 μL (25 μL in each nare) of virus corresponding with approximately 10^{3.5} TCID₅₀ by intranasal inoculation. Unused material was frozen at −20° C. to −80° C. for back titration.

### Laboratory Analysis

Inoculum was back titrated and the actual dose of the virus administered was verified by titrating replicate samples on Vero cells.

### Terminal Investigations

At the end of the study, on day 14, mice were euthanized by cervical dislocation. Gross necropsy was not performed.

### Data Analysis and Statistical Methods

Survival proportions at day 14, survival times and change in bodyweight (Area Under the Curve) were compared to the control group using Fisher's exact test, log-rank and Welch's t-test, respectively. All groups were compared to the vehicle (PBS solution) control group. P-values were adjusted according to Bonferroni (for two comparisons to vehicle) followed by a stepwise approach (starting with the highest antibody dose conditionally testing a lower dose if the previous step was statistically significant).

Statistical analysis was performed using R and statistical significance was set at α=0.05.

### Survival-Intraperitoneal

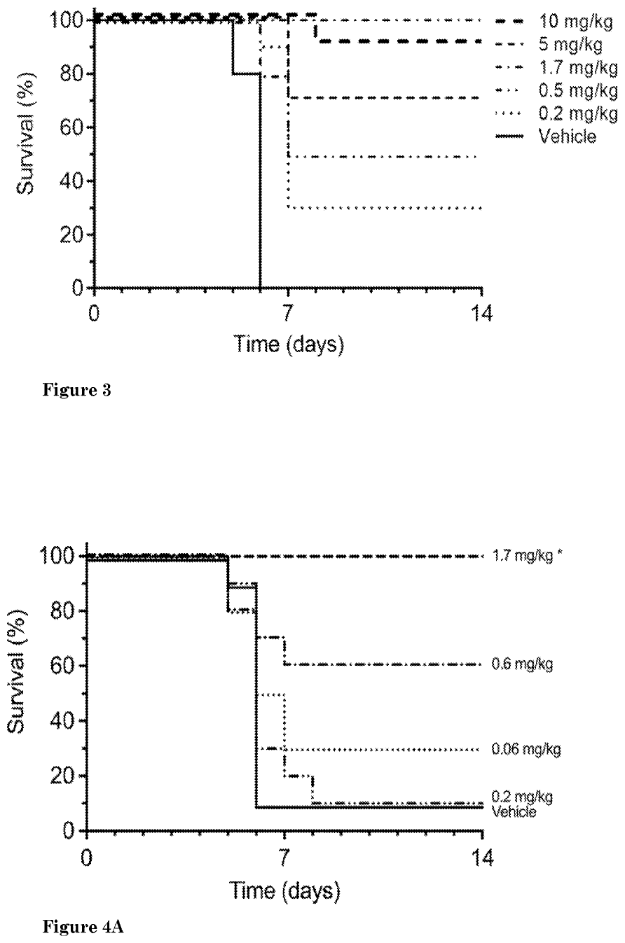

Prophylactic treatment with ≥1.7 mg/kg test antibody provides statistically significant increase in survival compared to the control group (See FIG. 3). The animals that were treated with 1.7 mg/kg test antibody had a 100% survival rate, with 5 mg/kg a rate of 70% and with 10 mg/kg 90% survival, whereas the survival proportion at day 6 in the control group was 0%. The median survival time of the control group was 6 days.

Prophylactic treatment with ≥0.2 mg/kg test antibody resulted in a significant improvement in survival time compared to the control group.

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline and the net AUC was defined as the summation of the area above and below the baseline using the percentage change per day. Prophylactic treatment with ≥0.5 mg/kg test antibody resulted in a significant reduction in weight loss compared to the control group.

### CONCLUSION

In this lethal SARS-COV-2 Delta K18 hACE2 Tg mouse model, the prophylactic intraperitoneal administration of ≥1.7 mg/kg test antibody provides significant improvement in survival compared to control and a reduction in weight loss whereas all the animals in the control group did not survive.

### Example 4: Antibody According to the Invention (Intraperitoneal Administration Study (I.P.))

The aim of this study was to assess the pre-exposure efficacy of a monoclonal antibody according to the invention having a heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574, following systemic intraperitoneal administration in a SARS-COV-2 Delta K18 hACE2 Tg mouse model.

Animals (n=10 per group) were treated with an intraperitoneal dose range of the test antibody described above (1.7 to 0.06 mg/kg mg/kg) or phosphate buffered saline (PBS) control, as vehicle control, at study Day-1. At study Day 0, animals were challenged with 10^{3.5} TCID₅₀ of SARS-COV-2 Delta variant.

### Materials and Methods

The monoclonal test antibody described above, at 20.41 mg/ml in 20 mM NaAC, 75 mM NaCl, 5% Sucrose, pH 5 was diluted to the final concentrations for administration (1.7 to 0.06 mg/kg in 100 μL), thereby preparing the test antibody dilution.

The test antibody dilution was made up at various concentrations so that 100 μL administrations to an assumed average weight of 20 g per mouse resulted in test antibody doses ranging from 1.7 mg/kg, 0.6 mg/kg, 0.2 mg/kg to 0.06 mg/kg.

The antibody was stored at −80° C.±10° C. and dilutions were stored at 4° C. until usage and the temperature of the storage unit was monitored.

The virus tested was strain SARS-COV-2 Delta variant (hCOV-19/Lelystad/B1.617.2/PW/2021, lineage AY.5) and passaged once on VERO-TMPRSS2.

### Animals

The animal species used was the SARS-COV-2 Delta K18 hACE2 Tg (B6.Cg-Tg (K18-ACE2) 2Prlmn/J (IMSR_JAX: 034860) mouse model, at a weight of c. 18-20 g. 50 mice were used at the age of 7 weeks and were exclusively female. Ten animals were allocated to 4 treatment groups to create groups with similar mean weight.

Mice were given a shelter as a place to hide and received nesting material to build a nest for sleeping and thermoregulation. Commercially available feed pellets and potable water were available ad libitum. Water was tested annually for contaminants. Standard laboratory animal breeding diet, RMH-B from Altromin, Germany was provided. Air temperature and relative humidity in the animal room were continuously monitored and recorded daily. The room was maintained under negative pressure and both incoming and outgoing air is filtered by HEPA filters.

Each animal was weighed daily beginning at the day of infection (Day 0).

### Study Design

The dose level of test antibody applied in this study was based on dose ranges from previous experiences. A total number of 50 mice, 7 weeks of age, were allocated to 5 experimental groups according to Table 4. Mice were given a period of >3 days for acclimatization.

TABLE 4
Intraperitoneal experimental detail

| Test | | Admin. | Dose | Day of | No. of |
|---|---|---|---|---|---|
| group | Antibody | Route | (mg/kg) | admin. | Animals |
| 1.7 | As | I.P. | 1.7 | −1 | 10 |
| 0.6 | disclosed | I.P. | 0.6 | −1 | 10 |
| 0.2 | above. | I.P. | 0.2 | −1 | 10 |
| 0.06 | | I.P. | 0.06 | −1 | 10 |
| Vehicle | — | I.P. | 0 | −1 | 10 |
| control | | | | | |

Female SARS-COV-2 Delta K18 hACE2 mice were treated via the intraperitoneal route of administration with the antibody at a dose between 1.7 mg/kg and 0.06 mg/kg. On Day 0, all mice were challenged with a lethal dose (10^{3.5} TCID₅₀ per mouse) of the SARS-COV-2 Delta variant and observed for weight loss and mortality until the end of the study at Day 14.

### Antibody Administration

The test antibody was stored at −80° C.±10° C. upon arrival. The appropriate dose, according to the treatment schedule (Table 4), was formulated based on an average weight of 20 g per mouse.

Mice in the treatment and vehicle control group received the indicated dose by intraperitoneal administration of 100 μL of antibody solution into the intraperitoneal cavity.

### Virus Administration

The virus material was stored at −80° C.±10° C. and was defrosted prior to administration, at a titer of 5.6 log₁₀ TCID₅₀ per mL. Once defrosted, the material was diluted in sterile PBS corresponding to approximately 10^{3.5} TCID₅₀ per 50 μL. As required, the animals were anesthetized with 0.2 mL of ketamine/xylazine mixture. Each animal received approximately 50 μL (25 μL in each nare) of virus dilution. Unused material was frozen at −20° C. to −80° C. for back titration.

### Laboratory Analysis

Inoculum was back titrated, and the dose of the virus administered was verified by titrating replicate samples on Vero E6 cells.

### Terminal Investigations

At the end of the study, on Day 14, mice were euthanized by cervical dislocation. Gross necropsy was not performed.

### Data Analysis and Statistical Methods

Survival proportions at day 14, survival times and change in bodyweight (Area Under the Curve) were compared to the control group using Fisher's exact test, log-rank and Welch's t-test, respectively. All groups were compared to the vehicle (PBS solution) control group. P-values were adjusted using a stepwise approach (starting with the highest antibody dose conditionally testing a lower dose if the previous step was statistically significant).

Statistical analysis was performed using R and statistical significance was set at α=0.05.

### Survival—Intraperitoneal

The vehicle control group showed 0% survival with a median survival time of 6 days. Intraperitoneal administration of 1.7 mg/kg test antibody provides statistically significant increase in survival compared to the control group, with 100% survival at this dose (See FIG. 4A).

Prophylactic treatment with 1.7 mg/kg test antibody resulted in a significant increase in survival time compared to the control group.

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline and the net AUC was defined as the summation of the area above and below the baseline using the percentage change per day. Prophylactic treatment with 1.7 mg/kg test antibody was required for significant reduction in body weight loss compared to the control group (FIG. 4B).

### CONCLUSION

In this lethal SARS-COV-2 Delta K18 hACE2 Tg mouse model, the prophylactic intraperitoneal administration of 1.7 mg/kg test antibody provides significant improvement in survival and a reduction in weight loss compared to control, whereas all the animals in the control group did not survive (see FIG. 4A).

### Example 5: Antibody According to the Invention

The aim of this study was to assess the pre-exposure efficacy of a monoclonal antibody according to the invention, having a heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574, following intranasal administration in a SARS-COV-2 Delta K18 hACE2 Tg mouse model.

Animals (n=10 per group) were treated with an intranasal dose range of the test antibody described above (10 to 0.2 mg/kg) or PBS control at study day −1. At study day 0, animals were challenged with 10^{3.5} TCID₅₀ of SARS-COV-2 Delta variant.

### Materials and Methods

The monoclonal antibody according to the invention was buffered in phosphate-buffered saline PBS was diluted to the final concentrations for administration (10 to 0.2 mg/kg in 50 μL)

The monoclonal dilution was made up at various concentrations so that 50 μL administrations to the mean weight per cage of the dosing group resulted in test antibody doses ranging from 10 mg/kg, 5 mg/kg, 1.7 mg/kg, 0.5 mg/kg to 0.2 mg/kg.

The antibody was stored at −80° C.±10° C. and dilutions were stored at 4° C. until usage and the temperature of the storage unit was monitored.

The virus strain tested was SARS-COV-2 Delta variant (Aus/VIC/18440/2021 (B.1.617.2), which had been passed three times through Vero-hSLAM cells.

### Animals

The animal species used was the SARS-COV-2 Delta K18 hACE2 Tg mouse model, at a weight of c. 16.4-21.8 g on the day of test article administration. 60 mice were used at the age of 7-11 weeks and were exclusively female. Ten animals were allocated to 6 treatment groups based on creating groups with similar mean weight based on Day-5 weights. All mice were housed in individually ventilated cages (IVCs), with corn cob bedding, tissues or shredded paper as nesting material, a wooden chew block, accessible food pellets and acidic water for animal nourishment and red plastic tunnels. Each cage system holds 3 to 5 mice per cage. All mice were transferred to fresh cages every 14 days. Food and water were inspected daily and topped up as needed. Each animal was weighed daily beginning at the day of infection (Day 0).

### Study Design

The dose level of the monoclonal antibody applied in the current example was based on the maximal dosing possible for the material stock concentration (4 mg/mL) and based on literature and previous experience with an anti-S2 antibody (CV3-25) showing protection IN at doses ≥1.7 mg/kg. A total number of 60 mice, 7-11 weeks of age were transported to the animal facility and allocated to 6 experimental groups according to Table 5 (see below). Mice were given a period of >3 days for acclimatization.

TABLE 5
Intranasal experimental detail

| Test | | Admin. | Dose | Day of | No. of |
|---|---|---|---|---|---|
| group | Antibody | route | (mg/kg) | admin. | Animals |
| 10 | As disclosed | I.N. | 10 | −1 | 10 |
| 5 | above. | I.N. | 5 | −1 | 10 |
| 1.7 | | I.N. | 1.7 | −1 | 10 |
| 0.5 | | I.N. | 0.5 | −1 | 10 |
| 0.2 | | I.N. | 0.2 | −1 | 10 |
| Vehicle | — | I.N. | 0 | −1 | 10 |

Female SARS-COV-2 Delta K18 hACE2 mice were treated via the intranasal route of administration with the antibody at a dose between 10 mg/kg and 0.2 mg/kg based upon group average mouse weight per cage. On Day 0, all mice were challenged with a lethal dose (10^{3.5} TCID₅₀) of the SARS-COV-2 Delta variant and observed for survival and weight loss until the end of the study at day 14.

### Antibody Administration

The test antibody was stored at −80° C.±10° C. upon arrival. The appropriate dose, according to the treatment schedule (Table 5), was formulated based upon the average weight per cage.

Just prior to dosing, the material was drawn into a 1 ml syringe with a 26G needle, and then administered to each mouse. Mice were anaesthetized with isoflurane (4% v/v with 2 L/min O₂) for two minutes. The mice were then scruffed and held at a +45 deg angle. 50 μL of compound or vehicle control was then slowly administered to the nares of each mouse (25 μL each nare). The mice were held for an additional 5 to10 seconds to ensure compound delivery to the lower respiratory tract before being put back into the home box.

### Virus Administration

The virus material was stored at −80° C.±10° C. and was defrosted prior to administration. Once defrosted, the material was diluted in cold PBS corresponding to approximately 10^{3.5} TCID₅₀/50 μL. As required, the animals were anesthetized by isoflurane (4% v/v with 2 L/min O₂) and each animal received approximately 50 μL (25 μL each nare) of virus corresponding with approximately 10^{3.5} TCID₅₀ by intranasal inoculation. Unused material was frozen at −20° C. to −80° C. for back titration.

### Laboratory Analysis

Inoculum was back titrated and the dose of the virus administered was verified by titrating replicate samples on Vero cells.

### Terminal Investigations

At the end of the study, on day 14, mice were euthanized by cervical dislocation. Gross necropsy was not performed.

### Data Analysis and Statistical Methods

Survival proportions at day 14, survival times and change in bodyweight (Area Under the Curve) were compared to the control group using Fisher's exact test, log-rank and Welch's t-test, respectively. All groups were compared to the vehicle (PBS solution) control group. P-values were adjusted according to Bonferroni (for two comparisons to vehicle) followed by a stepwise approach (starting with the highest antibody dose conditionally testing a lower dose if the previous step was statistically significant).

Statistical analysis was performed using R and statistical significance was set at α=0.05.

### Survival—Intranasal

Prophylactic treatment with ≥0.2 mg/kg test antibody provides statistically significant protection compared to the control group (See FIG. 5). The median survival time of the control group was 6 days.

Prophylactic treatment with ≥0.2 mg/kg test antibody resulted in a significant improvement in survival time compared to the control group.

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline and the net AUC was defined as the summation of the area above and below the baseline using the percentage change per day. Prophylactic treatment with >0.2 mg/kg test antibody resulted in a significant reduction in weight loss compared to the control group.

### CONCLUSION

In this lethal SARS-COV-2 Delta K18 hACE2 Tg mouse model, the prophylactic intranasal administration of ≥0.2 mg/kg test antibody provides significant improvement in survival and a reduction in weight loss compared to control. In contrast, all the animals in the control group had died by day 6.

### Example 6: Antibody According to the Invention (Intranasal Administration (I.N.))

The aim of this study was to assess the pre-exposure efficacy of a monoclonal antibody according to the invention, having a heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574, following intranasal administration in a SARS-COV-2 Delta K18 hACE2 Tg mouse model.

In this study, animals (n=10 per group, except n=9 for 0.6 mg/kg) were treated with an intranasal dose range of the test antibody described above (1.7 to 0.06 mg/kg) or PBS control as vehicle at study Day-1. At study Day 0, animals were challenged with 10^{3.5} TCID₅₀ of SARS-COV-2 Delta variant.

### Materials and Methods

The monoclonal test antibody described above, buffered at 20.41 mg/ml in 20 mM NaAC, 75 mM NaCl, 5% Sucrose, pH 5 was diluted to the final concentrations for administration (1.7 to 0.06 mg/kg in 50 μL), thereby preparing the test antibody dilution.

The test antibody dilution was made up at various concentrations so that 50 μL administrations to an assumed average weight of 20 g per mouse resulted in test antibody doses ranging from 1.7 mg/kg, 0.6 mg/kg, 0.2 mg/kg to 0.06 mg/kg.

The antibody was stored at −80° C.±10° C. and dilutions were stored at 4° C. until usage and the temperature of the storage unit was monitored.

The virus strain tested was SARS-COV-2 Delta variant (hCOV-19/Lelystad/B1.617.2/PW/2021, lineage AY.5) and passaged once on VERO-TMPRSS2.

### Animals

The animal species used was the SARS-COV-2 Delta K18 hACE2 Tg (B6.Cg-Tg (K18-ACE2) 2Prlmn/J (IMSR_JAX: 034860) mouse model, at a weight of c. 18-20 g. 49 mice were used at the age of 7 weeks and were exclusively female. Ten animals (n=9 for 0.6 mg/kg group) were allocated to 5 treatment group to create groups with similar mean weight.

Mice were given a shelter as a place to hide and received nesting material to build a nest for sleeping and thermoregulation. Commercially available feed pellets and potable water were available ad libitum. Water was tested annually for contaminants. Standard laboratory animal breeding diet, RMH-B from Altromin, Germany was provided. Air temperature and relative humidity in the animal room were continuously monitored and recorded daily. The room was maintained under negative pressure and both incoming and outgoing air was filtered by HEPA filters.

Each animal was weighed daily beginning at the day of infection (Day 0).

### Study Design

The dose level of test antibody applied was based on previous experiences with the antibody according to invention in an K18-hACE2 mouse model. A total number of 49 mice, 7 weeks of age, were allocated to 5 experimental groups according to Table 6. Mice were given a period of >3 days for acclimatization.

TABLE 6
Intranasal experimental detail

| Test | | Admin. | Dose | Day of | No. of |
|---|---|---|---|---|---|
| group | Antibody | Route | (mg/kg) | admin. | Animals |
| 1.7 | As disclosed | I.N. | 1.7 | −1 | 10 |
| 0.6 | above. | I.N. | 0.6 | −1 | 9 |
| 0.2 | | I.N. | 0.2 | −1 | 10 |
| 0.06 | | I.N. | 0.06 | −1 | 10 |
| Vehicle | — | I.N. | 0 | −1 | 10 |
| control | | | | | |

Female SARS-COV-2 Delta K18 hACE2 mice were treated via the intranasal route of administration with the antibody at a dose between 1.7 mg/kg and 0.06 mg/kg at Day −1. On Day 0, all mice were challenged with a lethal dose (10^{3.5} TCID₅₀ per mouse) of the SARS-COV-2 Delta variant and observed for weight loss and mortality until the end of the study at Day 14.

### Antibody Administration

The test antibody was stored at −80° C.±10° C. upon arrival. The appropriate dose, according to the treatment schedule (Table 6), was formulated based on an average weight of 20 g per mouse.

Mice in the treatment and vehicle control group were anaesthetized with 0.2 mL of ketamine/xylazine mixture prior to antibody administration. Animals received the indicated dose by intranasal administration of 25 μL of antibody solution into each nare (50 μL per mouse) using a sterile pipette tip.

### Virus Administration

The virus material was stored at −80° C.±10° C. and was defrosted prior to administration, at a titer of 5.6 log₁₀ TCID₅₀ per mL. Once defrosted, the material was diluted in sterile PBS corresponding to approximately 10^{3.5} TCID₅₀ per 50 μL. As required, the animals were anesthetized with 0.2 mL of ketamine/xylazine mixture. Each animal received approximately 50 μL (25 μL in each nare) of virus dilution using a sterile pipette tip. Unused material was frozen at −20° C. to −80° C. for back titration.

### Laboratory Analysis

Inoculum was back titrated, and the dose of the virus administered was verified by titrating replicate samples on Vero E6 cells.

### Terminal Investigations

At the end of the study, on Day 14, mice were euthanized by cervical dislocation. Gross necropsy was not performed.

### Data Analysis and Statistical Methods

Survival proportions at Day 14, survival times and change in bodyweight (Area Under the Curve) were compared to the control group using Fisher's exact test, log-rank and Welch's t-test, respectively. All groups were compared to the vehicle (PBS solution) control group. P-values were adjusted using a stepwise approach (starting with the highest antibody dose conditionally testing a lower dose if the previous step was statistically significant).

Statistical analysis was performed using R and statistical significance was set at α=0.05.

### Survival—Intranasal

For the study, the vehicle control group showed 0% survival with median survival times of 6 days. Prophylactic intranasal treatment with ≥0.2 mg/kg test antibody provides statistically significant increase in survival compared to the control group (See FIG. 6A).

Prophylactic intranasal treatment with ≥0.06 mg/kg test antibody resulted in a significant increase in survival time compared to the control group (FIG. 6A).

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline and the net AUC was defined as the summation of the area above and below the baseline using the percentage change per day. Prophylactic intranasal treatment with ≥0.06 mg/kg of test antibody resulted in a significant reduction in weight loss compared to the control group (FIG. 6B).

### CONCLUSION

In this lethal SARS-COV-2 Delta K18 hACE2 Tg mouse model, the prophylactic intranasal administration of ≥0.2 mg/kg test antibody provided significant increase in survival and with ≥0.06 mg/kg a reduction of body weight loss compared to the control group (FIG. 6). In contrast, all the animals in the control group did not survive.

### Example 7: Antibody According to the Invention

The aim of this study is to assess the pre- and post-exposure efficacy of a monoclonal antibody according to the invention, having a heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574, following intranasal administration in a NHP model in which each subject is pre-screened for the absence of serum antibodies against SARS-CoV-2 viruses.

NHP subjects are treated with an intranasal dose of the composition comprising the antibody described above. The flat dose, or nominal dose, of the antibody administered to each NHP subject is between 0.01 mg and 10 mg (e.g. between 0.01 to 2.0 mg (“Low Dose”) or between 2.0 to 10 mg (“High Dose”)) or Control (e.g. vehicle only) at study day −1.

### Materials and Methods

The monoclonal antibody according to the invention is provided in a sodium acetate buffer (e.g. 20 mM Sodium Acetate, 75 mM Sodium Chloride, 5% sucrose, pH 5.5) and is diluted to the final concentration for administration ranging from 0.01 mg to 2.0 mg or 2.0 to 10 mg nominal dose, administered as 50 μL to 500 μL per nare.

The NHP subjects allocated to the Control Group (Sodium acetate buffer) are administered with vehicle (e.g. 20 mM Sodium Acetate, 75 mM Sodium Chloride, 5% sucrose, pH 5.5), administered as 50 μL to 500 μL per nare.

All NHPs receive intranasal challenge with SARS-COV-2 at day 0.

Samples are taken during the study, sample types include nasal sampling (e.g. nasal swabs, wash or scrapes), serum and/or bronchoalveolar lavage (BAL) samples. Sampling period includes study duration, in a time frame between day −1 to the end of the study (e.g. 7 to 21 days follow-up period).

Samples are analyzed as required for viral quantification (by RT-PCR and/or TCID₅₀) and/or antibody titers are analyzed by ELISA, HPLC or similar method.

### Animals

The NHP subjects are used at a weight of between 3 to 12 kg (e.g. 5 kg per animal) on commencement of the study. Between 4 and 12 animals are allocated to each treatment group.

### Study Design

The dose level of the monoclonal antibody applied in the current example is based on the maximal dosing and a lower dose based upon allometric calculations obtained from murine studies.

TABLE 7
Intranasal experimental detail per antibody

| Test | | Admin. | Dose | Day of | No. of |
|---|---|---|---|---|---|
| group | Antibody | route | (mg) | admin. | Animals |
| High dose | As disclosed | I.N. | 2.0-10 | −1 | 4-12 |
| Low dose | above. | I.N. | 0.01-2.0 | −1 | 4-12 |
| Vehicle | — | I.N. | 0 | −1 | 4-12 |

The NHP subjects receive intranasal administrations with compositions comprising the antibody at a dose between 0.01 mg to 2.0 mg or 2.0 mg to 10 mg (Nominal Dose). On Day 0, all NHP subjects are challenged with a dose of the SARS-COV-2 and observed for clinical manifestations (e.g. weight loss and/or temperature) and virology measurements until the end of the study (e.g. Day 7 to 21).

### Antibody Administration

The test antibody or vehicle only is administered to each nare (e.g. pipette or spray) according to the treatment schedule (Table 7), using volumes of between 50 μL to 500 μL per nare.

### Virus Administration

The virus material is defrosted prior to administration. Once defrosted, the material is diluted and each animal receives between 50 μL to 500 μL of virus solution per nare. The NHP subjects are infected with between 10² and 10⁸ TCID₅₀ of SARS-COV-2 (e.g. 10⁵ TCID₅₀ of SARS-COV-2).

### Data Analysis and Statistical Methods

Clinical manifestations (e.g. body weight loss and temperature) and virological measurements are used for comparisons between treatment and Control Group.

### Results—Clinical Manifestations

Prophylactic treatment with the Low Dose and High Dose antibody administration provides reduction in clinical manifestation measurements, compared to the Control Group.

### Results—Virological Measurements

Prophylactic treatment with the Low Dose and High Dose antibody administration provides reduction in virological measurements, compared to the Control Group. All Control Group NHPs show viral loads in samples after SARS-COV-2 challenge.

### CONCLUSION

In this SARS-COV-2 NHP model, the prophylactic intranasal administration of 2.0 to 10 mg (High Dose) and 0.01 to 2.0 mg (Low Dose) of the test antibody according to the invention provides a reduction in virological measurements and/or a reduction in clinical manifestations as compared with Control Group NHPs.

### Example 8: Composition Comprising an Antibody According to the Invention

The aim of this study is to assess the pre- and post-exposure efficacy of a composition comprising a first monoclonal antibody according to the invention, having a heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574 (“First Antibody”), and a second monoclonal antibody according to the invention, having a heavy chain CDR1 as SEQ ID NO: 645, heavy chain CDR2 as SEQ ID NO: 646, heavy chain CDR3 as SEQ ID NO: 647, a light chain CDR1 as SEQ ID NO: 648, a light chain CDR2 as SEQ ID NO: 649, and a light chain CDR3 as SEQ ID NO: 650 (“Second Antibody”), following intranasal administration in a NHP model in which each subject is pre-screened for the absence of serum antibodies against SARS-COV-2 viruses.

NHP subjects are treated with an intranasal dose of the composition comprising the antibodies described above (single antibodies or combinations of both antibodies). The flat dose, or nominal dose, of the antibodies administered to each NHP subject is between 0.003 mg and 5 mg or Control (e.g. vehicle only) at study day −1.

### Materials and Methods

The monoclonal antibody according to the invention is provided in a sodium acetate buffer (e.g. 20 mM Sodium Acetate, 75 mM Sodium Chloride, 5% sucrose, pH 5.5) and is diluted to the final concentration for administration ranging from 0.01 mg and 20 mg nominal dose, administered as 50 μL to 500 μL per nare.

The NHP subjects allocated to the Control Group (Sodium acetate buffer) are administered with vehicle (e.g. 20 mM Sodium Acetate, 75 mM Sodium Chloride, 5% sucrose, pH 5.5), administered as 50 μL to 500 μL per nare.

All NHPs receive intranasal challenge with SARS-COV-2 at day 0.

Samples are taken during the study, sample types include nasal sampling (e.g. nasal swabs, wash or scrapes), serum and/or bronchoalveolar lavage (BAL) samples. Sampling period includes study duration, in a time frame between day −1 to the end of the study (e.g. 7 to 21 days follow-up period).

Samples are analyzed as required for viral quantification (by RT-PCR and/or TCID₅₀) and/or antibody titers by ELISA or similar methods.

### Animals

The NHP subjects are used at a weight of between 3 to 12 kg (e.g. 5 kg per animal) on commencement of the study. Between 4 and 12 animals are allocated to each treatment group.

### Study Design

The dose level of the monoclonal antibody and the combination applied in the current example is based on the maximal dosing and based upon allometric calculations obtained from murine studies.

TABLE 8
Intranasal experimental detail

| Treatment | | Admin. | Dose | Day of | No. of |
|---|---|---|---|---|---|
| group | Antibody | route | (mg) | admin. | Animals |
| 1 | First Antibody only | I.N. | 0.003-5 | −1 | 4-12 |
| 2 | Second Antibody only | I.N. | 0.003-5 | −1 | 4-12 |
| 3 | First and Second | I.N. | 0.003-5 | −1 | 4-12 |
| | Antibody | | | | |
| Vehicle | — | I.N. | 0 | −1 | 4-12 |

The NHP subjects receive intranasal administrations with compositions comprising either single antibody nominal doses of between 0.003-5 mg (Treatment Groups 1 or 2). Different NHP subjects receive intranasal administrations with compositions comprising a mixture of the First and Second Antibody at nominal doses between 0.003-5 mg (Treatment Group 3). On Day 0, all NHP subjects are challenged with a dose of the SARS-CoV-2 and observed for clinical manifestations (e.g. weight loss and/or temperature) and virology measurements until the end of the study (e.g. Day 7 to 21).

### Antibody Administration

The antibody compositions (Treatment Groups 1 to 3) or vehicle only are administered to each nare (e.g. pipette or spray) according to the treatment schedule (Table 8), using volumes of between 50 μL to 500 μL per nare.

### Virus Administration

The virus material is defrosted prior to administration. Once defrosted, the material is diluted and each animal receives between 50 μL to 500 μL of virus solution per nare. The NHP subjects are infected with between 10² and 10⁸ TCID₅₀ of SARS-COV-2 (e.g. 10⁵ TCID₅₀ of SARS-COV-2).

### Data Analysis and Statistical Methods

Clinical manifestations (e.g. body weight loss and temperature) and virological measurements are used for comparisons between treatments (e.g. Treatment Groups 1 to 3) and Control Group and/or comparison within treatment groups (e.g. Treatment Group 1 against Treatment Group 3).

### Results—Clinical Manifestations

Prophylactic treatment with treatments (as indicated in Table 8) provides reduction in clinical manifestation measurements, compared to the Control Group or compared within treatment groups.

### Results—Virological Measurements

Prophylactic treatment with treatments (as indicated in Table 8) provides reduction in virological measurements, compared to the Control Group or compared within treatment groups.

### CONCLUSION

In this SARS-COV-2 NHP model, the prophylactic intranasal administration of treatments (as indicated in Table 8) according to the invention provides a reduction in clinical manifestations and/or a reduction in virological measurements, as compared with control group NHPs and within treatments.

### Example 9: Bi-Specific Antibody According to the Invention

The aim of this intranasal murine study is to assess the pre- and post-exposure efficacy of a bispecific monoclonal antibody in comparison with the parental antibodies, according to the invention, having a first Fab capable of binding to the stem helix of SARS-COV-2 and a second Fab capable of binding to the fusion peptide of SARS-COV-2. The first parental antibody (“Parental mAb 1”) has the following CDRs as described in SEQ ID Numbers: 055, 137, 257, 363, 480 and 574. The second parental antibody (“Parental mAb 2”) has the following CDRs as described in SEQ ID Numbers: 645, 646, 647, 648, 649 and 650. The bi-specific antibody according to the invention has a first Fab that comprises a sequence comprising any one or more of SEQ ID Numbers: 055, 137, 257, 363, 480 or 574; the second Fab comprises a sequence comprising any one or more of SEQ ID Numbers: 645, 646, 647, 648, 649 to 650.

Animals are treated with an intranasal nominal dose (0.001 mg/kg to 15 mg/kg) comprising either Parental mAb 1, or Parental mAb 2, or the bispecific antibody described above, or vehicle control at study day −1.

### Materials and Methods

The monoclonal parental and bispecific antibodies according to the invention is provided in a sodium acetate buffer (e.g. 20 mM Sodium Acetate, 75 mM Sodium Chloride, 5% sucrose, pH 5.5) and is diluted to the final concentration for administration ranging from 0.001 mg/kg to 15 mg/kg, administered as 25 μL to 50 μL per nare.

The mice allocated to the control group (Sodium acetate buffer) are administered with vehicle (e.g. 20 mM Sodium Acetate, 75 mM Sodium Chloride, 5% sucrose, pH 5.5), administered as 25 μL to 50 μL per nare.

All mice receive intranasal challenge with SARS-COV-2 Delta at day 0.

### Animals

The SARS-COV-2 Delta K18 hACE2 Tg mice are used at a weight of c. 15 g to 22 g (e.g. 20 g per animal) on commencement of the study. Between 6 and 10 animals are allocated to each treatment group.

### Study Design

The dose level of the monoclonal and bispecific antibodies applied in the current example is based on the maximal dosing and based upon previous results from efficacy studies in mice with the parental antibodies alone. From the maximal dose of ≤15 mg/kg, a 3- or 4-fold dilution curve for each the parental or bispecific antibody is applied.

TABLE 9
Intranasal experimental detail per antibody

| Test | | Dose | Day of | No. of |
|---|---|---|---|---|
| group | Antibody | (mg/kg) | admin. | Animals |
| 1 | Parental mAb 1 | 0.001-15 | −1 | 6-10 |
| | (dose 1) | (e.g. 0.0027, 0.063) | | |
| 2 | Parental mAb 1 | 0.001-15 | −1 | 6-10 |
| | (dose 2) | (e.g. 0.106, 0.189) | | |
| 3 | Parental mAb 1 | 0.001-15 | −1 | 6-10 |
| | (dose 3) | (e.g. 0.425, 0.567) | | |
| 4 | Parental mAb 1 | 0.001-15 | −1 | 6-10 |
| | (dose 4) | (e.g. 1.7) | | |
| 5 | Parental mAb 2 | 0.001-15 | −1 | 6-10 |
| | (dose 1) | (e.g. 0.0027, 0.063) | | |
| 6 | Parental mAb 2 | 0.001-15 | −1 | 6-10 |
| | (dose 2) | (e.g. 0.106, 0.189) | | |
| 7 | Parental mAb 2 | 0.001-15 | −1 | 6-10 |
| | (dose 3) | (e.g. 0.425, 0.567) | | |
| 8 | Parental mAb 2 | 0.001-15 | −1 | 6-10 |
| | (dose 4) | (e.g. 1.7) | | |
| 9 | Bispecific mAb | 0.001-15 | −1 | 6-10 |
| | (dose 1) | (e.g. 0.007, 0.005) | | |
| 10 | Bispecific mAb | 0.001-15 | −1 | 6-10 |
| | (dose 2) | (e.g. 0.027, 0.020) | | |
| 11 | Bispecific mAb | 0.001-15 | −1 | 6-10 |
| | (dose 3) | (e.g. 0.106, 0.080) | | |
| 12 | Bispecific mAb | 0.001-15 | −1 | 6-10 |
| | (dose 4) | (e.g. 0.425, 0.310) | | |
| 13 | Bispecific mAb | 0.001-15 | −1 | 6-10 |
| | (dose 5) | (e.g. 1.7, 1.25) | | |
| 14 | Bispecific mAb | 0.001-15 | −1 | 6-10 |
| | (dose 6) | (e.g. 5.0) | | |
| Vehicle | — | 0 | −1 | 6-10 |

The mice are treated via the intranasal route of administration with compositions comprising either parental antibodies or the bispecific antibody at dose ranges between 0.001 and 15 mg/kg. On Day 0, all mice are challenged with a dose of the SARS-COV-2 and observed for survival and body weight until the end of the study (e.g. Day 10-15).

### Antibody Administration

The parental monoclonal antibodies or bispecific antibody or vehicle only is administered to each nare (e.g. pipette or spray) according to the treatment schedule (Table 9), using volumes of between 25 μL to 50 μL per nare.

### Virus Administration

The virus material is defrosted prior to administration. Once defrosted, the material is diluted and each animal received between 25 μL to 50 μL of virus per nare. The mice are infected with between 10² and 10⁸ TCID₅₀ of SARS-COV-2 (e.g. 10^{3.5} TCID₅₀ of SARS-COV-2).

### Laboratory Analysis

Inoculum is returned to the lab and the dose of the virus administered is verified by titrating replicate samples on Vero cells.

### Clinical Monitoring

General health observations are performed on each animal from the day of arrival until the end of the study at least once daily (during normal servicing procedures). Each animal is weighed daily beginning one day prior to infection (day −1).

### Data Analysis and Statistical Methods

Survival proportions and survival times and change in bodyweight (Area Under the Curve) are compared to the corresponding control group using Fisher's exact test, log-rank and Welch's t-test, respectively. All groups are compared to the vehicle control group. P values are adjusted according to Bonferroni (for three comparisons to vehicle) followed by a stepwise approach within antibody (starting with the highest dose and conditionally testing a lower dose if the previous step was statistically significant).

Survival dose response curves are fitted for each of the treatments (Parental mAb 1, Parental mAb 2 and Bispecific) and the ED₅₀ is estimated. The efficacy of the bispecific antibody is then compared to the parental antibodies

### Survival

Prophylactic treatment with Parental mAb 1, Parental mAb 2 or bispecific antibody (as indicated in Table 9) compared to the control group provides statistically significant protection against mortality and a significant improvement in survival time compared to the control group.

### Body Weight

Change in bodyweight is analyzed using an Area Under the Curve (AUC) analysis in which the last observed body weight is carried forward if a mouse died/is euthanized during the study. The weight per mouse at day 0 is used as baseline and weight change is determined relative to baseline with the net AUC defined as the summation of the area above and below the baseline using the percentage change per day.

Prophylactic treatment with the parental or bispecific antibodies (as indicated in Table 9) provides a statistically significant reduction in weight loss, compared to the control group.

### CONCLUSION

In this SARS-COV-2 Delta mouse model, the prophylactic intranasal administration of a bispecific antibody (as indicated in Table 9) according to the invention provides significant improvement in survival and a reduction in body weight loss compared with Control Group. The outcome of the combination index is suggestive of at least a comparable, an additive or potentially synergistic effect for the bispecific compared to the parental antibodies.

### Example 10: Bi-Specific According to the Invention

The aim of this intranasal murine study was to assess the pre- and post-exposure efficacy of a bispecific monoclonal antibody in comparison with the parental antibodies, according to the invention, having a first Fab capable of binding to the stem helix of SARS-COV-2 and a second Fab capable of binding to the fusion peptide of SARS-COV-2. The first parental antibody (“Parental mAb 1”) has the following CDRs as described in SEQ ID Numbers: 055, 137, 257, 363, 480 and 574. The second parental antibody (“Parental mAb 2”) has the following CDRs as described in SEQ ID Numbers: 639, 640, 641, 642, 643 and 644. The bi-specific antibody according to the invention has a first Fab that comprises a sequence comprising any one or more of SEQ ID Numbers: 055, 137, 257, 363, 480 or 574; the second Fab comprises a sequence comprising any one or more of SEQ ID Numbers: 639, 640, 641, 642, 643 and 644.

Animals (n=8 per group) were treated with an intranasal dose comprising either 0.007 mg/kg to 1.7 mg/kg (Parental mAb 1 or Parental mAb 2), or 0.002 mg/kg to 10 mg/kg (bispecific antibody) described above, or vehicle control at study Day-1.

### Materials and Methods

The monoclonal parental antibodies were provided in a sodium acetate buffer (20 mM Sodium Acetate, 75 mM Sodium Chloride, 5% sucrose, pH 5.5) and the bispecific antibody according to the invention was provided in phosphate buffered saline (PBS) and all antibodies were diluted to the final concentration for administration ranging from either 0.007 mg/kg to 1.7 mg/kg (Parental mAb 1 or Parental mAb 2), or 0.002 mg/kg to 10 mg/kg (bispecific antibody), administered as 25 μL per nare.

The mice allocated to the control group were administered with vehicle (phosphate buffered saline, PBS), administered as 25 μL per nare.

All mice receive intranasal challenge with SARS-COV-2 Delta at day 0.

### Animals

The animal species used was the SARS-COV-2 Delta K18 hACE2 Tg (B6.Cg-Tg (K18-ACE2) 2Prlmn/JArc; Jax™ Stock No.: 034860) mouse model, at a weight of c. 13.6-22.6 g at day 0. A total of 144 mice were used at the age of 7-11 weeks and were exclusively female. Eight animals were allocated to 17 treatment groups to create groups with similar mean weight. Eight mice we allocated to a vehicle control group. All mice were housed in individually ventilated cages (IVCs), with corn cob bedding, tissues or shredded paper as nesting material, a wooden chew block, accessible food pellets and acidic water for animal nourishment and red plastic tunnels. Each cage system holds 3 to 5 mice per cage. All mice were transferred to fresh cages every 14 days. Food and water were inspected daily and topped up as needed. Each animal was weighed daily beginning at the day of infection (day 0).

### Study Design

The dose level of the parental antibodies and the bispecific thereof applied in the current example was based on previous experience with the parental antibodies intranasally showing protection with intranasal administration IN at doses ≥0.2 mg/kg (parental mAb 1) and ≥0.5 mg/kg (parental mAb 2). The potency of the bispecific antibody of the two parental antibodies was expected to be similar, and one dose step up and down from the dose range for the parental antibodies was included for the bispecific antibody. A total number of 144 mice, 7-11 weeks of age, were allocated to 18 experimental groups according to Table 10 (see below). Mice were given a period of >3 days for acclimatization.

TABLE 10
Intranasal experimental detail per parental
antibody and bispecific antibody

| Test | | Dose | Day of | No. of |
|---|---|---|---|---|
| group | Antibody | (mg/kg) | admin. | Animals |
| 1 | Parental mAb 1 | 1.7 | −1 | 8 |
| 2 | Parental mAb 1 | 0.43 | −1 | 8 |
| 3 | Parental mAb 1 | 0.11 | −1 | 8 |
| 4 | Parental mAb 1 | 0.03 | −1 | 8 |
| 5 | Parental mAb 1 | 0.007 | −1 | 8 |
| 6 | Parental mAb 2 | 1.7 | −1 | 8 |
| 7 | Parental mAb 2 | 0.43 | −1 | 8 |
| 8 | Parental mAb 2 | 0.11 | −1 | 8 |
| 9 | Parental mAb 2 | 0.03 | −1 | 8 |
| 10 | Parental mAb 2 | 0.007 | −1 | 8 |
| 11 | Bispecific mAb | 10 | −1 | 8 |
| 12 | Bispecific mAb | 2.5 | −1 | 8 |
| 13 | Bispecific mAb | 0.63 | −1 | 8 |
| 14 | Bispecific mAb | 0.16 | −1 | 8 |
| 15 | Bispecific mAb | 0.039 | −1 | 8 |
| 16 | Bispecific mAb | 0.010 | −1 | 8 |
| 17 | Bispecific mAb | 0.002 | −1 | 8 |
| Vehicle | — | 0 | −1 | 8 |
| control | | | | |

The mice were treated via the intranasal route of administration with compositions comprising either parental antibodies or the bispecific antibody at dose ranges between 0.002 and 10 mg/kg. On Day 0, all mice were challenged with a dose of the SARS-COV-2 and observed for survival and body weight until the end of the study (Day 14).

### Antibody Administration

The test antibody was stored at −80° C.±10° C. upon arrival. The appropriate dose, according to the treatment schedule (Table 10), was formulated based upon the average weight per cage.

Just prior to dosing, the material prepared according to Table 10 was drawn into a 1 mL syringe with a 26G needle, and then administered to each mouse. Mice were anaesthetized with isoflurane (4% v/v with 2 L/min O₂) for two minutes. The mice were then scruffed and held at a +45 deg angle. 50 μL of the material or vehicle control was then slowly administered to the nares of each mouse (25 μL each nare). The mice were held for an additional 5 to 10 seconds to ensure the compound delivery to the lower respiratory tract before being put back into the home box.

### Virus Administration

The virus material was stored at −80° C.±10° C. and was defrosted prior to administration. Once defrosted, the material was diluted in cold PBS corresponding to approximately 10^{3.5} TCID₅₀/50 μL. As required, the animals were anesthetized by isoflurane (4% v/v with 2 L/min O₂) and each animal received approximately 50 μL (25 μL each nare) of virus corresponding with approximately 10^{3.5} TCID₅₀ by intranasal inoculation.

### Terminal Investigations

At the end of the study, on Day 14, mice were euthanized by cervical dislocation. Gross necropsy was not performed.

### Data Analysis and Statistical Methods

Survival proportions at day 14, survival times and change in bodyweight (using the Area Under the Curve) were compared to the vehicle control group using a Fisher's exact test, log-rank and Welch's t-test, respectively. P-values were adjusted according to Bonferroni for four comparisons against the vehicle control group, followed by a stepwise approach, within arm, starting with the highest dose and conditionally testing a lower dose if the previous dose was statistically significant.

Survival proportions at day 14 were used to estimate the survival dose response curve using Probit regression without assuming a parallel dose response curve for the treatments. The model was used to estimate the median effective dose and the ratio between the treatments was determined.

Statistical analysis was performed using R, Probit regression was performed using the R package drc (v 3.2.0), and statistical significance was set at α=0.05.

### Survival

The vehicle control group showed 0% survival with a median survival time of 7 days. Prophylactic intranasal treatment with ≥0.11 mg/kg of either parental antibody intranasally provides statistically significant increase in survival compared to control group (See FIGS. 7A and D). Prophylactic treatment with ≥0.16 mg/kg of the bispecific antibody provides statistically significant increase in survival compared to control group (FIG. 7G).

Prophylactic intranasal treatment with ≥0.03 mg/kg of either parental antibody or ≥0.039 mg/kg of the bispecific antibody intranasally provides statistically significant increase in survival times compared to the control group (See FIGS. 7A, D and G).

Survival dose response curves

The survival dose response curves were estimated using Probit regression without assuming a parallel dose response curve between treatments. The vehicle control group was included in the modelling at a dose of 0 mg/kg. The Probit regression model was used to estimate the antibody dose corresponding to 50% survival (ED₅₀) (see Table 11). The intranasal median effective dose was lowest for the bispecific antibody, with 0.013 mg/kg

TABLE 11
Estimated ED₅₀'s (in mg/kg, rounded to 3 decimals).

| | ED₅₀ (mg/kg, 95% |
|---|---|
| Treatment | confidence interval) |
| Parental mAb 1 | 0.023 (0.005-0.104) |
| Parental mAb 2 | 0.046 (0.012-0.169) |
| Bispecific antibody | 0.013 (0.004-0.040) |

### Body Weight

Change in bodyweight was analyzed using an Area Under the Curve (AUC) analysis in which the last observed body weight was carried forward if a mouse died/was euthanized during the study. The weight per mouse at day 0 was used as baseline and weight change was determined relative to baseline with the net AUC defined as the summation of the were a above and below the baseline using the percentage change per day.

Prophylactic intranasal treatment with ≥0.039 mg/kg of bispecific antibody (FIG. 7H) or ≥0.03 mg/kg of the parental antibodies (FIGS. 7B and E) resulted in a significant reduction in weight loss compared to the control group.

### CONCLUSION

In this SARS-COV-2 Delta mouse model, the prophylactic intranasal administration of a bispecific antibody (as indicated in Table 11 and FIG. 7G) according to the invention provides significant increases in survival compared with control group at doses ≥0.16 mg/kg.

### Example 11: Composition Comprising Antibody Combination According to the Invention

The aim of this intranasal murine study is to assess the pre- and post-exposure efficacy of a composition comprising a first antibody according to the invention capable of binding to the stem helix of SARS-COV-2, and second antibody capable of binding to the fusion peptide of SARS-COV-2. The first antibody (“First mAb”) having a heavy chain CDR1 as SEQ ID NO: 055, heavy chain CDR2 as SEQ ID NO: 137, heavy chain CDR3 as SEQ ID NO: 257, a light chain CDR1 as SEQ ID NO: 363, a light chain CDR2 as SEQ ID NO: 480, and a light chain CDR3 as SEQ ID NO: 574. The second antibody (“Second mAb”) having a heavy chain CDR1 as SEQ ID NO: 645, heavy chain CDR2 as SEQ ID NO: 646, heavy chain CDR3 as SEQ ID NO: 647, a light chain CDR1 as SEQ ID NO: 648, a light chain CDR2 as SEQ ID NO: 649, and a light chain CDR3 as SEQ ID NO: 650.

Animals are treated with an intranasal dose (0.001 mg/kg to 15 mg/kg) of the comprising either First mAb, or Second mAb, or the composition comprising the first and second antibody described above, or vehicle control at study day −1.

### Materials and Methods

The First mAb and Second mAb are provided in a sodium acetate buffer (e.g. 20 mM Sodium Acetate, 75 mM Sodium Chloride, 5% sucrose, pH 5.5) and is diluted to the final concentration for administration ranging from 0.001 mg/kg to 20 mg/kg, administered as 25 μL to 50 μL per nare.

The mice allocated to the control group (Sodium acetate buffer) are administered with vehicle (e.g. 20 mM Sodium Acetate, 75 mM Sodium Chloride, 5% sucrose, pH 5.5), administered as 25 μL to 50 μL per nare.

All mice receive intranasal challenge with SARS-COV-2 Delta at day 0.

### Animals

The SARS-COV-2 Delta K18 hACE2 Tg mice are used at a weight of c. 15 g to 22 g (e.g. 20 g per animal) on commencement of the study. Between 6 and 10 animals are allocated to each treatment group.

### Study Design

The dose level of the First mAb, or Second mAb, or the composition comprising the first and second antibody described above in the current example is based on the maximal dosing and based upon previous results from efficacy studies in mice with the parental antibodies alone. From the maximal dose of ≤15 mg/kg, a 3- or 4-fold dilution curve for each the parental or bispecific antibody is applied.

TABLE 12
Intranasal experimental detail per antibody

| Test | | Dose | Day of | No. of |
|---|---|---|---|---|
| group | Antibody | (mg/kg) | admin. | Animals |
| 1 | First mAb 1 | 0.001-15 | −1 | 6-10 |
| | (dose 1) | (e.g. 0.0027, 0.063) | | |
| 2 | First mAb 1 | 0.001-15 | −1 | 6-10 |
| | (dose 2) | (e.g. 0.106, 0.189) | | |
| 3 | First mAb 1 | 0.001-15 | −1 | 6-10 |
| | (dose 3) | (e.g. 0.425, 0.567) | | |
| 4 | First mAb 1 | 0.001-15 | −1 | 6-10 |
| | (dose 4) | (e.g. 1.7) | | |
| 5 | Second mAb 2 | 0.001-15 | −1 | 6-10 |
| | (dose 1) | (e.g. 0.0027, 0.063) | | |
| 6 | Second mAb 2 | 0.001-15 | −1 | 6-10 |
| | (dose 2) | (e.g. 0.106, 0.189) | | |
| 7 | Second mAb 2 | 0.001-15 | −1 | 6-10 |
| | (dose 3) | (e.g. 0.425, 0.567) | | |
| 8 | Second mAb 2 | 0.001-15 | −1 | 6-10 |
| | (dose 4) | (e.g. −1.7) | | |
| 9 | Combination mAb | 0.001-15 | −1 | 6-10 |
| | (dose 1) | (e.g. 0.007, 0.005) | | |
| 10 | Combination mAb | 0.001-15 | −1 | 6-10 |
| | (dose 2) | (e.g. 0.027, 0.020) | | |
| 11 | Combination mAb | 0.001-15 | −1 | 6-10 |
| | (dose 3) | (e.g. 0.106, 0.080) | | |
| 12 | Combination mAb | 0.001-15 | −1 | 6- 10 |
| | (dose 4) | (e.g. 0.425, 0.310) | | |
| 13 | Combination mAb | 0.001-15 | −1 | 6-10 |
| | (dose 5) | (e.g. 1.7, 1.25) | | |
| 14 | Combination mAb | 0.001-15 | −1 | 6-10 |
| | (dose 6) | (e.g. 5.0) | | |
| Vehicle | — | 0 | −1 | 6-10 |

The mice are treated via the intranasal route of administration with compositions comprising either First or Second antibodies or the composition comprising the combination at dose ranges between 0.001 and 15 mg/kg. On Day 0, all mice are challenged with a dose of the SARS-COV-2 and observed for survival and body weight until the end of the study (e.g. Day 10-15).

### Antibody Administration

The First or Second antibodies or the composition comprising the combination or vehicle only is administered to each nare (e.g. pipette or spray) according to the treatment schedule (Table 12), using volumes of between 25 μL to 50 μL per nare.

### Virus Administration

The virus material is defrosted prior to administration. Once defrosted, the material is diluted and each animal received between 25 μL to 50 μL of virus per nare. The mice are infected with between 10² and 10⁸ TCID₅₀ of SARS-COV-2 (e.g. 10^{3.5} TCID₅₀ of SARS-COV-2).

### Laboratory Analysis

Inoculum is returned to the lab and the dose of the virus administered is verified by titrating replicate samples on Vero cells.

### Clinical Monitoring

General health observations are performed on each animal from the day of arrival until the end of the study at least once daily (during normal servicing procedures). Each animal is weighed daily beginning one day prior to infection (day −1).

### Data Analysis and Statistical Methods

Survival proportions and survival times and change in bodyweight (Area Under the Curve) are compared to the control group using Fisher's exact test, log-rank and Welch's t-test, respectively. All groups are compared to the vehicle control group. P values are adjusted according to Bonferroni (for three comparisons to vehicle) followed by a stepwise approach within antibody (starting with the highest dose and conditionally testing a lower dose if the previous step was statistically significant).

### Combination Index

Survival dose response curves are fitted for each of the treatments (First or Second antibodies or the composition comprising the combination) and the ED₅₀ is estimated. The efficacy of the combination is then compared to the First and Second antibodies by calculation of the combination index.

### Survival

Prophylactic treatment with the First or Second antibodies or the composition comprising the combination (as indicated in Table 12) compared to the control group provides statistically significant protection against mortality and a significant improvement in survival time compared to the control group.

### Body Weight

Change in bodyweight is analyzed using an Area Under the Curve (AUC) analysis in which the last observed body weight is carried forward if a mouse died/is euthanized during the study. The weight per mouse at day 0 is used as baseline and weight change is determined relative to baseline with the net AUC defined as the summation of the area above and below the baseline using the percentage change per day.

Prophylactic treatment with the composition comprising the combination (as indicated in Table 12) provides a statistically significant reduction in weight loss, compared to the control group.

### CONCLUSION

In this SARS-COV-2 Delta mouse model, the prophylactic intranasal administration of the composition comprising the combination (as indicated in Table 12) according to the invention provides significant improvement in survival and a reduction in body weight loss compared with Control Group. The outcome of the combination index is suggestive of at least a comparable, an additive or potentially synergistic effect for the combination compared to the First or Second antibodies administered alone.

### Example 12: ELISA Binding Assessment of an Antibody According to the Invention

The objective of this study was to assess the binding of the antibody against different Corona Spike antigens, a variety of alpha and beta coronaviruses.

Binding was tested in an enzyme-linked immunosorbent assay (ELISA). Briefly, a Spike antigen was used to coat the surface of a microwell of an ELISA plate. The antibody was then added in decreasing concentration to the coated and pre-blocked microwell surface in duplicates, attaching to the coated antigen. A detection antibody was added, labeled with horse radish peroxide (HRP), that then generates a colorimetric signal upon addition of the substrate. The readout was optical density and reflects binding. The antibody was assayed in duplicate against a number of alpha and beta coronavirus spike antigens, including but not limited to SARS-COV-2 Delta, Omicron XBB.1.5 and human coronavirus NL63. The antibody was tested in comparison to other anti-S1 antibodies in the range of 10.0-0.000002 nM. Half maximal effective concentration (EC₅₀) was calculated for each antibody on each antigen as an estimate of binding affinity.

TABLE 13
ELISA EC50 values (nM) for the antibody according
to invention. N/D = no EC₅₀ value was able
to be determined, because it was below the tested range.

| | CoV-2 | CoV-2 | |
|---|---|---|---|
| | Delta | XBB.1.5 | NL63 |
| Parental mAb 1 | 0.00033 | 0.000075 | N/D |
| Anti-S1 antibody | 6.9 | N/D | N/D |
| DZIF-10c | | | |
| Anti-S1 antibody | 0.010 | N/D | N/D |
| ImdevimAb | | | |
| Human IgG1 | N/D | N/D | N/D |
| Isotype | | | |

The ELISA binding assay (Table 13) showed that the antibody according to invention was able to effectively bind all tested Spike antigens from beta coronaviruses, including SARS-CoV-2 Delta and Omicron XBB.1.5. The alpha coronavirus NL63 was not bound by the antibody. The anti-S1 antibodies tested only bound the SARS-COV-2 Delta, as expected from literature and did not show breadth amongst alpha coronavirus NL63. The isotype control antibody did not show any binding to the coronavirus antigens.

### Example 13: Affinity Binding Assessment by MSD of an Antibody According to the Invention

The objective of this study was to assess relative binding affinity against Spike antigens in a multiplexed way, with very high specificity and low sample input. Therefore, an antibody was tested in a multiplexed assay, Meso Scale Discovery (MSD), which uses electrochemiluminescent labels that were conjugated to detection antibodies. In this assay, up to ten trimeric viral antigen are separately printed onto a spot each well of the plate, allowing for a multiplexed assay readout. The antibody according to the invention bind the viral antigens through Fab-mediated recognition, then an anti-human IgG Sulfo-Tag detection antibody is added, recognizing human IgG Fab. Upon addition of read buffer containing substrate, and running a current through the plate electrodes, an electro-(current) chemi-(buffer substrate) luminescent (light) cascade is initiated which results in light emission. The intensity of emitted light is measured per spot, revealing bound levels of analyte. The antibody was assayed in duplicate against Spike antigens of SARS-CoV-2, SARS-COV-1, MERS, hCoV NL63, hCoV HKU, hCoV OC43 and hCoV 229E (V-Plex COVID-19 Coronavirus Panel 3 (IgG) Kit cat #K15399U-2) (Table 15), as well as a set of Omicron variants (V-Plex SARS-COV-2 Panel 34 (IgG) Kit cat #K15690U-2) in a separate assay (Table 15. The antibody was tested in a dilution range starting from 555 ng/ml for the Panel 3 and 10 ng/ml in the Omicron Panel 24 kit. Light emission from the MSD sulfo-tag antibody was quantified with MSD Discovery workbench, calibration curves were used to calculate antibody concentrations, by fitting the raw electrochemiluminescent units (eCLU) from the calibrators to a logistic regression for curve fitting (Sigmoidal, 4PL, X=conc), and the model asymptotes were constrained to the lower limit of detection (LLOQ) and the upper limit of detection (ULOQ). The concentration (pg/ml) corresponding to the midpoint of the dynamic range (ie the range between the lower and upper limit of quantification, LLOQ and ULOQ) was reported.

TABLE 14
Antibody concentrations corresponding to the midpoint of the dynamic range (LLOQ +
ULOQ)/2 (ng/ml) for the antibody according to the invention against different
alpha and beta coronaviruses. N/D = antibody concentration could not be determined.

| | | | | | | | | SARS- |
|---|---|---|---|---|---|---|---|---|
| | | | | | | | | CoV-2 |
| | SARS- | SARS- | MERS- | hCoV- | hCoV- | hCoV- | hCoV- | S1 |
| | CoV-2 | CoV-1 | CoV | OC43 | HKU1 | NL63 | 229E | RBD |
| Parental | 0.40 | 0.21 | 0.16 | 5.17 | 7.63 | N/D | 44.48 | 30.05 |
| mAb 1 | | | | | | | | |
| Anti-S1 mAb | 0.16 | N/D | N/D | N/D | N/D | N/D | N/D | 0.09 |
| DZIF-10c | | | | | | | | |
| isotype | N/D | N/D | N/D | N/D | N/D | N/D | N/D | N/D |
| control | | | | | | | | |

The MSD binding array (see Table 14) showed that the antibody according to invention was able to effectively bind all Spike antigens from beta coronaviruses, including SARS-CoV-2, MERS, HKU1 and OC43. Alpha coronavirus NL63 was not bound by the antibody, whereas alpha coronavirus 229E was bound at high concentrations. Other hCoV beta coronaviruses HKU1 and OC43 were bound effectively by the antibody according to invention, indicating breadth among beta coronaviruses including MERS and seasonal cold coronaviruses OC43 and HKU1 and limited breadth among alpha coronaviruses for 229E. The SARS-COV-2 receptor binding domain (RBD) on the S1 part of the Spike was only bound at very high concentrations by the antibody according to invention. The anti-S1 antibody tested here bound the RBD domain and the wildtype SARS-COV-2 at low concentrations, as expected from literature and did not show breadth amongst other coronaviruses. The isotype control antibody did not show any binding to the coronavirus antigens.

TABLE 15
Antibody concentrations corresponding to the midpoint of the dynamic range (LLOQ +
ULOQ)/2 (pg/ml) for the antibody according to the invention against different SARS-
CoV-2 Omicron viruses is shown. N/D: antibody concentration could not be determined.

| | | SARS- | SARS- | SARS- | SARS- | SARS- | SARS- | SARS- | SARS- | SARS- |
|---|---|---|---|---|---|---|---|---|---|---|
| | SARS- | CoV-2 | CoV-2 | CoV-2 | CoV-2 | CoV-2 | CoV-2 | CoV-2 | CoV-2 | CoV-2 |
| | CoV-2 | BA.1 | BA.2.75 | BA.5 | BF.7 | BN.1 | BQ.1 | BQ.1.1 | XBB.1 | XBB.1.5 |
| Parental | 694.84 | 124.02 | 168.37 | 188.1 | 153.13 | 121.58 | 206.87 | 184.13 | 196.04 | 200.03 |
| mAb 1 | | | | | | | | | | |
| Anti-S1 | 117 | 93 | 81 | 630 | 13625 | 11558 | N/D | N/D | N/D | N/D |
| mAb | | | | | | | | | | |
| DZIF-10c | | | | | | | | | | |
| isotype | N/D | N/D | N/D | N/D | N/D | N/D | N/D | N/D | N/D | N/D |
| control | | | | | | | | | | |

The MSD binding array showed that the antibody according to invention can effectively bind all Omicron variants tested in this assay, at even lower concentrations than Wuhan (Table 15), this also aligns with Omicron XBB.1.5 binding results from the ELISA assay (Table 14). The SARS-COV-2 BA.1 and BN.1 variants were bound at especially low concentrations indicating that the antibody according to the invention retains binding efficacy for all Omicron variants tested. While the anti-S1 control antibody bound SARS-CoV-2 Spike at lower concentrations than the antibody according to invention, the earlier Omicron mutants were only bound at very high concentrations and the later Omicron mutants such as BQ.1 and XBB.1 were not bound. The isotype control antibody did not show any binding to the coronavirus antigens.

### Example 14: Live Virus Neutralization of an Antibody According to the Invention

The objective of this study was to assess the ability of an antibody to neutralize live corona viruss. The antibody was tested for functional activity in a live virus microneutralization assay against MERS, SARS-COV-1 and SARS-COV-2 Wuhan. Briefly, dilution series of an antibody were pre-incubated with the corresponding virus and then added to the respective cell line (Vero: MERS and SARS-COV-1, SARS-COV-2: Vero E6 cells) in quadruplicates. After incubation, cells were fixed, stained with an anti-nucleocapsid antibody, and an enzymatically tagged detection antibody was added. Colored precipitate signaling nucleocapsid presence was read out via an Immunospot analyzer and 50% inhibitory concentration (IC₅₀), was reported via the Zielinska method (REF: https://doi.org/10.1186/1743-422X-2-84). The antibody was tested for neutralizing activity in the range of 0.025 to 500 μg/ml.

TABLE 16
Live virus neutralization assay. IC₅₀ values in μg/ml were displayed
for the antibody according to invention against three different
beta coronaviruses. A pooled convalescent serum from SARS-CoV-2
patients was used as a comparator and SARS-CoV-2 positive
control, MN₅₀ titers are shown. N/D = not determined

| | SARS-CoV-2 | | |
|---|---|---|---|
| Live virus neutralization | Wuhan | SARS-CoV-1 | MERS-CoV |
| Parental mAb 1 | 9.87 | 2.73 | 1.49 |
| SARS-CoV-2 pooled | 142.00 | N/D | N/D |
| convalescent serum | | | |

Overall, the antibody was able to neutralize the tested viruses SARS-COV-1, SARS-COV-2 and MERS at varying concentrations (see Table 16), with the lowest IC₅₀ concentrations for MERS-COV, therefore confirming that the antibody according to the invention can neutralize these different beta coronaviruses, as suggested by the binding assessment via MSD (Table 15a). The positive serum control for SARS-COV-2 shows a high MN₅₀ titer.

### Example 15: Pseudovirion Neutralization of an Antibody According to the Invention

The objective of this study was to assess the ability of an antibody to neutralize different pseudotyped virus particles. The advantage over a live virus assay was that pseudotyped viruses can be used at a lower biosafety level laboratory and new variants of concern can be produced and tested more rapidly, allowing for broader testing across variants of concern or interest. Briefly, pseudovirions were produced by co-transfecting virus expression plasmids with the pHIV-1NL43 AEnv-NanoLuc reporter virus plasmid in HEK293T cells. Then, dilution series of the antibody were pre-incubated with the corresponding pseudovirus and then added to HEK 293T cells expressing ACE2, the entry receptor for SARS-COV-2, SARS-COV-1 and NL63. For hCoV 229E, the diluted antibody mixed with virus was added to Huh7 cells which express the aminopeptidase N (APN receptor which facilitates entry of hCoV 229E into host cells. After incubation, cells were washed and lysed to measure the luciferase activity in cell lysates using the Nano-Glo Luciferase Assay System and GloMax system, with a readout of relative light units (RLUs). The 50% inhibitory concentrations (IC₅₀) were determined as the antibody concentration at which infectivity was inhibited by 50% using a four Parameter Logistic Regression (4PL) curve fit. The bispecific antibody was tested for neutralizing activity in the range of 0.0042 to 250 μg/ml against SARS-COV-1, SARS-COV-2 Wuhan, Delta, Omicron BA4, Omicron BQ.1.1, Omicron XBB.1, hCoV NL63 and hCoV 229E.

TABLE 17
Pseudovirion neutralization assay. IC₅₀ values in ug/ml were displayed for
the anti-stem helix antibody against different alpha and beta coronaviruses.
For multiple runs, the geometric mean with 95% normal range is shown.

| | | SARS- | SARS- | SARS- | SARS- | SARS- | | |
|---|---|---|---|---|---|---|---|---|
| | SARS- | CoV-2 | CoV-2 | CoV-2 | CoV-2 | CoV-2 | CoV | CoV |
| | CoV-1 | Wuhan | Delta | BA4 | XBB.1 | BQ.1.1 | 229E | NL63 |
| Parental | 0.588 | 0.072 | 2.858 | 4.134 | 4.851 | 4.921 | >250 | >250 |
| mAb | (0.088- | (0.054- | (1.503- | (3.583- | (2.613- | (2.833- | | |
| 1 | 3.925) | 0.097) | 5.436) | 4.769) | 9.005) | 8.547) | | |

Overall, the antibody was able to neutralize the tested beta viruses SARS-COV-1, SARS-CoV-2 including variants of concern at varying concentrations (see Table 17), with an overall trend of higher IC₅₀ concentrations for the Omicron variants. The antibody did not neutralize alpha coronaviruses hCoV NL63 and 229E, as expected based on the lack of binding in the MSD assay for NL63. While 229E was bound in the MSD assay (Table 16a, the activity did not translate to detectable neutralizing activity in this pseudotyped neutralization assay.

### Example 16: Epitope Mapping of an Antibody According to the Invention

The goal of this study was to precisely determine the amino acid sequence that the antibody binds to in the Spike protein across different alpha and beta coronaviruses. The method includes mapping of linear epitopes using libraries of overlapping synthetic peptides from the spike protein. Briefly, linear epitopes of the sequence of interest were synthesized directly on a solid chip, generating a library of linear mimics, aiding identification of the correct amino acid sequence of the target antibody. The library of peptides consists of overlapping 15-amino acid fragments, with an overlap of 14 amino acids. Binding of the antibody to each of the synthesized peptide chips was tested in an enzyme-linked immunosorbent assay (ELISA) assay via incubation of the peptide arrays with antibody solutions. After washing, peptide arrays were incubated with an antibody peroxidase conjugate and substrate was added, and the reaction was then stopped using hydrogen peroxide. Color development was measured, and intensity was reported.

The antibody bound the peptide array of alpha and beta coronaviruses in a specific epitope of a 10-20 amino acids (see FIG. 16). The method allowed for a high confidence in the epitope definition, with certain variance in the outer were as of the epitope, indicated by asterisk for the respective strain.

### Example 17: Affinity Binding Assessment by MSD of a Bispecific Antibody

The objective of this study was to assess relative binding affinity against Spike antigens in a multiplexed way, with very high specificity and low sample input. Therefore, the bispecific antibody compared to the parental antibodies was tested in a multiplexed assay, Meso Scale Discovery (MSD), which uses electrochemiluminescent labels that were conjugated to detection antibodies.

The first parental antibody (“Parental mAb 1” has the following CDRs as described in SEQ ID Numbers: 055, 137, 257, 363, 480 and 574. The second parental antibody (“Parental mAb 2”) has the following CDRs as described in SEQ ID Numbers: 639, 640, 641, 642, 643 and 644. The bi-specific antibody according to the invention has a first Fab that comprises a sequence comprising any one or more of SEQ ID Numbers: 055, 137, 257, 363, 480 or 574; the second Fab comprises a sequence comprising any one or more of SEQ ID Numbers: 639, 640, 641, 642, 643 and 644.

Three versions of bispecific antibodies have been produced with the same production technology allowing for slightly varying formats, and then tested, all with the combination of the Parental mAb 1 and Parental mAb 2.

Bispecific 1 (●) (according to the invention) is described, inter alia, in Clause 26 and also in Clause 29 and Clause 30, with a first arm heavy chain amino acid sequence as described in SEQ ID Number: 663 and a light chain amino acid sequence as described in SEQ ID Number: 664, and a second arm having a heavy chain amino acid sequence as described in SEQ ID Number: 665 and a light chain amino acid sequence as described in SEQ ID Number: 666.

Bispecific 2 (⋄) (according to the invention) is described, inter alia, in Clause 26 and also in Clause 31 and Clause 32, with a first arm heavy chain amino acid sequence as described in SEQ ID Number: 667 and a light chain amino acid sequence as described in SEQ ID Number: 668, and a second arm having a heavy chain amino acid sequence as described in SEQ ID Number: 669 and a light chain amino acid sequence as described in SEQ ID Number: 670.

Bispecific 3 (□) (according to the invention) is described, inter alia, in Clause 26 and also in Clause 33, with a first arm heavy chain amino acid sequence as described in SEQ ID Number: 667 and a light chain amino acid sequence as described in SEQ ID Number: 668, and a second arm having a heavy chain amino acid sequence as described in SEQ ID Number: 671 and a light chain amino acid sequence as described in SEQ ID Number: 672. In this assay, up to ten trimeric viral antigens are separately printed onto a spot each well of the plate, allowing for a multiplexed assay readout. The parental antibodies and bispecific antibody bind the viral antigens through Fab-mediated recognition, then an anti-human IgG Sulfo-Tag detection antibody is added, recognizing human IgG Fab. Upon addition of read buffer containing substrate, and running a current through the plate electrodes, an electro-(current) chemi-(buffer substrate) luminescent (light) cascade is initiated which results in light emission. The intensity of emitted light is measured per spot, revealing bound levels of analyte. The antibodies were assayed in duplicate against Spike antigens of SARS-COV-2, SARS-COV-1, MERS, hCoV NL63, hCOV HKU, hCoV OC43 and hCoV 229E (V-Plex COVID-19 Coronavirus Panel 3 (IgG) Kit cat #K15399U-2) and a set of Omicron variants (V-Plex SARS-COV-2 Panel 34 (IgG) Kit cat #K15690U-2) in a separate assay. The parental antibodies and bispecific antibodies were tested in a range of 0.25 to 555 ng/ml as a dilution curve, the dilution range was adapted to include a wider range in light of possible reduced potency of the bispecific antibody. Light emission from the MSD sulfo-tag antibody was quantified with MSD Discovery workbench, calibration curves were used to back-calculate antibody concentrations, by fitting the raw electrochemiluminescent units (eCLU) from the calibrators to a logistic regression for curve fitting (Sigmoidal, 4PL, X=conc), and the model asymptotes were constrained to the lower limit of detection (LLOQ) and the upper limit of detection (ULOQ). The concentration (ng/ml) corresponding to the midpoint of the dynamic range (ie the range between the lower and upper limit of quantification, LLOQ and ULOQ) was reported.

TABLE 18
Antibody concentrations corresponding to the midpoint of the dynamic
range (LOQ + ULOQ)/2 (ng/ml) for the bispecific antibodies compared
to the parental antibodies against different alpha and beta coronaviruses.
N/D: indicates that antibody concentration was not detected.

| | | | | | | | | SARS- |
|---|---|---|---|---|---|---|---|---|
| | | | | | | | | CoV-2 |
| | SARS- | SARS- | MERS- | hCoV- | hCoV- | hCoV- | hCoV- | S1 |
| | CoV-2 | CoV-1 | CoV | OC43 | HKU1 | NL63 | 229E | RBD |
| Parental mAb 1 | 0.40 | 0.21 | 0.16 | 5.17 | 7.63 | N/D | 44.48 | 30.05 |
| Parental mAb 2 | 6.73 | 7.00 | 2.77 | 65.11 | 92.04 | 0.70 | 5.44 | N/D |
| Bispecific 1 | 1.10 | 0.70 | 0.43 | 10.64 | 14.10 | 2.98 | 22.36 | 30.19 |
| Bispecific 2 | 1.10 | 0.68 | 0.42 | 10.73 | 16.46 | 2.43 | 20.83 | 40.55 |
| Bispecific 3 | 1.19 | 0.80 | 0.48 | 10.11 | 16.01 | 2.58 | 23.12 | 25.70 |
| Anti-S1 mAb | 0.16 | N/D | N/D | N/D | N/D | N/D | N/D | 0.09 |
| DZIF-10c | | | | | | | | |

The MSD binding array (see Table 18) showed that the bispecific antibodies were able to effectively bind all Spike antigens from alpha and beta coronaviruses. Table 18 reports the binding assessment of Parental mAb 1, Parental mAb 2, three constructed bispecific antibodies (according to the invention), and an anti-S1 monoclonal antibody against a variety of virus representatives from the genera α-coronaviruses and β-coronaviruses. These data are presented in FIG. 17A.

Alpha coronaviruses NL63 was not bound by the parental anti-stem helix antibody Parental mAb 1, but only by the anti-fusion peptide antibody and the bispecific antibodies, albeit at slightly higher concentrations than the parental antibody. Alpha coronavirus 229E was bound at high concentrations by Parental mAb 1 and at lower concentrations by the Parental mAb 2. Other hCoV beta coronaviruses HKU1 and OC43 were bound effectively by the Parental mAb 1 and at higher concentrations also by Parental mAb 2, whereas the bispecific bound at intermediate concentrations, suggesting Fab leverage of the Parental mAb 1. The SARS-COV-2 Spike RBD antigen was only bound at high concentrations by the Parental mAb 1 (stem-helix mAb) and the bispecific antibodies. The control anti-S1 mAb only bound SARS-COV-2 Spike and RBD, at low concentrations. The data suggest that the bispecific antibodies retain the binding capacity from each parental antibody, here demonstrated by the ability to utilize the Fab arm of the Parental mAb 2 and retaining alpha coronavirus binding activity, especially for NL63.

Ordinarily, one would expect an intermediate level of binding for a bispecific antibody, between that exhibited by Parental mAb 1 and Parental mAb 2. Of importance therefore is that Bispecific 1, Bispecific 2 and Bispecific 3 (all according to the invention) have acquired broader binding capabilities compared to the parental monoclonal antibodies, in so far as α-coronaviruses and ß-coronaviruses are concerned. Of particular importance, is that Bispecific 1, Bispecific 2 and Bispecific 3 have acquired the superior binding traits shown by Parental mAb 1 with respect to β-coronaviruses binding whilst also acquiring the superior binding traits from Parental mAb 2 with respect to the tested α-coronaviruses.

TABLE 19
Antibody concentrations corresponding to the midpoint of the dynamic range (LOQ + ULOQ)/2 (pg/ml) for
the bispecific antibodies compared to the parental antibodies against different SARS-CoV-2 Omicron variants.

| | | SARS- | SARS- | SARS- | SARS- | SARS- | SARS- | SARS- | SARS- | SARS- |
|---|---|---|---|---|---|---|---|---|---|---|
| | SARS- | CoV-2 | CoV-2 | CoV-2 | CoV-2 | CoV-2 | CoV-2 | CoV-2 | CoV-2 | CoV-2 |
| | CoV-2 | BA.1 | BA.2.75 | BA.5 | BF.7 | BN.1 | BQ.1 | BQ.1.1 | XBB.1 | XBB.1.5 |
| Parental mAb 1 | 842 | 169 | 229 | 263 | 238 | 182 | 303 | 269 | 215 | 321 |
| Parental mAb 2 | 7670 | 4349 | 10501 | 8726 | 10235 | 9696 | 10421 | 7638 | 6502 | 10134 |
| Bispecific 1 | 690 | 121 | 162 | 169 | 172 | 130 | 209 | 182 | 161 | 215 |
| Bispecific 2 | 1286 | 239 | 342 | 364 | 337 | 270 | 426 | 361 | 318 | 477 |
| LC0024 | 1173 | 196 | 281 | 304 | 275 | 233 | 365 | 308 | 269 | 394 |

The MSD binding array (see Table 19) showed that the bispecific antibodies were able to effectively bind all Spike antigens from SARS-COV-2 Omicron variants, set out in chronological order, left to right, starting with the original SARS-COV-2 strain. While only the Parental mAb 1 retains efficient binding against later omicron variants and the Parental mAb 2 would require high antibody concentrations, the bispecific antibodies retains binding, suggesting that the bispecific antibodies can leverage the Fab arm of Parental mAb 1.

FIG. 17B depicts the binding capabilities of Parental mAb 1 (▴) (according to the invention) and Parental mAb 2 (Δ), demonstrating the superior binding capabilities of the Parental mAb 1 for the original SARS-COV-2 strain and its subsequent SARS-COV-2 Omicron variants.

FIG. 17C retains the Parental mAb 1 (▴) (according to the invention) as a point of reference but instead focuses on the relative binding capabilities of the three constructed bispecific antibodies; Bispecific 1 (●), Bispecific 2 (⋄) and Bispecific 3 (□) for the original SARS-COV-2 strain and its subsequent SARS-COV-2 Omicron variants.

Acknowledging that Bispecific 2 and Bispecific 3 have broader binding capabilities compared to both parental mAbs, Bispecific 2 and Bispecific 3 both have excellent binding capabilities to the SARS-COV-2 Omicron variants, although these do not supersede those of parental mAb 1.

Using the highly sensitive MSD binding array, the inventors have surprisingly discovered and that not only does Bispecific 1 have broader binding capabilities as compared to both parental mAbs, it also consistently shows superior binding to both historic and recent SARS-COV-2 Omicron variants when compared to its parental mAbs and Bispecific 2 and Bispecific 3.

### Example 18: Live Virus Neutralization of a Bispecific Antibody

The objective of this study was to assess the ability of the bispecific antibody to neutralize live corona viruses and pseudovirions. The bispecific antibody was tested for functional activity in a live virus microneutralization assay against MERS, SARS-COV-1, SARS-CoV-2 Wuhan, Delta and Omicron BA.4/5.

The first parental antibody (“Parental mAb 1”) has the following CDRs as described in SEQ ID Numbers: 055, 137, 257, 363, 480 and 574. The second parental antibody (“Parental mAb 2”) has the following CDRs as described in SEQ ID Numbers: 639, 640, 641, 642, 643 and 644. The bi-specific antibody according to the invention has a first Fab that comprises a sequence comprising any one or more of SEQ ID Numbers: 055, 137, 257, 363, 480 or 574; the second Fab comprises a sequence comprising any one or more of SEQ ID Numbers: 639, 640, 641, 642, 643 and 644.

The bispecific antibody Bispecific 1 comprises the first Fab and the second Fab as described above.

Briefly, dilution series of antibodies were pre-incubated with the corresponding virus and then added to the respective cell line (Vero: MERS and SARS-COV-1, all other Vero E6 cells). After incubation, cells were fixed, stained with an anti-nucleocapsid antibody, and an enzymatically tagged and a detection antibody was added. Colored precipitate signaling nucleocapsid presence was read out via an Immunospot analyzer and 50% inhibitory concentration (IC₅₀), was reported. The bispecific antibody was tested for neutralizing activity in the range of 0.02 to 500 μg/ml compared to the individual control antibodies.

TABLE 20
Live virus neutralization assay. IC₅₀ values (in ug/ml) were determined
for three different beta coronaviruses. Parental antibodies were
assayed alongside the bispecific antibody as comparator.

| | SARS- | SARS- | SARS- | | |
|---|---|---|---|---|---|
| | CoV-2 | CoV-2 | CoV-2 | SARS- | MERS- |
| | Wuhan | Delta | BA.4/5 | CoV-1 | CoV |
| Bispecific 1 | 11.92 | 14.17 | 4.85 | 6.67 | 1.32 |
| Parental mAb 1 | 6.44 | 14.77 | 2.92 | 4.18 | 0.86 |
| Parental mAb 2 | 21.70 | 16.08 | 4.62 | 5.94 | 11.59 |

SARS-CoV-2 Wuhan, Delta, Omicron BA4/5, NQ.1 and XBB.1, hCoV NL63 and hCoV 229E.

Overall, the bispecific antibody was able to neutralize the tested viruses SARS-COV-1, SARS-COV-2 Wuhan, Delta and Omicron BA.4/5 and MERS at varying concentrations (see Table 20). The anti-stem helix parental antibody was able to neutralize SARS-COV-2 Wuhan and MERS at lower IC₅₀ concentrations compared to the anti-fusion peptide antibody, this trend was also observed for the bispecific antibody, indicating that the antibody can indeed leverage the anti-stem helix Fab arm. This indicates that the bispecific antibody retains affinity from both Fab arms and across tested beta coronaviruses.

### Example 19: Pseudovirion Neutralization of a Bispecific Antibody

The pseudovirion neutralization assay was performed against pseudotyped virus particles.

The first parental antibody (“Parental mAb 1”) has the following CDRs as described in SEQ ID Numbers: 055, 137, 257, 363, 480 and 574. The second parental antibody (“Parental mAb 2”) has the following CDRs as described in SEQ ID Numbers: 639, 640, 641, 642, 643 and 644. The bi-specific antibody according to the invention has a first Fab that comprises a sequence comprising any one or more of SEQ ID Numbers: 055, 137, 257, 363, 480 or 574; the second Fab comprises a sequence comprising any one or more of SEQ ID Numbers: 639, 640, 641, 642, 643 and 644.

The bispecific antibody Bispecific 1 comprises the first Fab and the second Fab as described above.

The advantage over a live virus assay was that pseudotyped viruses can be used at a lower biosafety level laboratory and new variants of concern can be produced and tested more rapidly. Briefly, pseudovirions were produced by co-transfecting virus expression plasmids with the pHIV-1NL43 AEnv-NanoLuc reporter virus plasmid in HEK293T cells. Then, dilution series of antibodies were pre-incubated with the corresponding pseudovirus and then added to HEK 293T cells expressing ACE2, the entry receptor for SARS-COV-2, SARS-COV-1 and NL63. After incubation, cells were washed and lysed to measure the luciferase activity in cell lysates using the Nano-Glo Luciferase Assay System and GloMax system. The 50% inhibitory concentrations (IC₅₀) were determined as the antibody concentration at which infectivity was inhibited by 50% using a four Parameter Logistic Regression (4PL) curve fit. The bispecific antibody was tested for neutralizing activity in the range of 0.01 to 250 μg/ml compared to the individual control antibodies against

TABLE 21
Pseudovirion neutralization assay. IC₅₀ values in ug/ml were displayed
for the anti-stem helix and anti- fusion peptide antibodies against
different alpha and beta coronaviruses. For multiple runs, the
geometric mean with 95% lower and upper range is shown.

| | | | CoV-2 | CoV-2 | CoV-2 |
|---|---|---|---|---|---|
| | CoV-2 | CoV-2 | Omicron | Omicron | Omicron |
| | Wuhan | Delta | BA4/5 | BQ.1 | XBB.1.1 |
| Bispecific 1 | 0.0006 | 0.307 | 5.14 | 8.09 | 11.9 |
| Parental | 0.072 | 2.858 | 4.134 | 4.921 | 4.851 |
| mAb 1 | (95% normal | (95% normal | (95% normal | (95% normal | (95% normal |
| | range; 0.088- | range; 1.503- | range; 3.583- | range; 2.833- | range; 2.613- |
| | 3.925) | 5.436) | 4.769) | 8.547) | 9.005) |
| Parental | 0.068 | 0.488 | 7.563 | 10.090 | 20.619 |
| mAb 2 | (95% normal | (95% normal | (95% normal | (95% normal | (95% normal |
| | range; 0.029- | range; 0.061- | range; 1.189- | range; 1.679- | range; 9.245- |
| | 0.160) | 3.882) | 48.096) | 60.640) | 45,987) |

The Pseudovirion neutralization assay (see Table 21) shows that for the Omicron variants, especially the ones arising later such as XBB.1.1, the Parental antibody 1 according to invention was able to neutralize at lower concentrations than the anti-fusion peptide Parental mAb 2, with the bispecific antibody retaining the neutralizing activity with IC₅₀ concentrations in the middle of each Parental mAb, except for SARS-COV-2 Wuhan, which was neutralized by the bispecific antibody at the lowest concentration compared to the Parental antibodies. This indicates that the bispecific antibody retains neutralizing affinity from each Fab arm of the parental antibodies.

### Example 20: Bispecific Antibody Validation

This bispecific binding competition assay demonstrates the ability of the bispecific antibodies according to the invention to bind to both the stem helix and the fusion peptide of a coronavirus, as exemplified using these two epitopes found on the SARS-COV-2 spike protein.

### 1. ANTIBODY PRODUCTION

Using a mammalian cell expression system, bispecific antibodies according to the invention are expressed and purified having a first Fab that binds to the stem helix of a SARS-COV-2 and a second Fab that binds to the fusion peptide of SARS-COV-2, for example wherein the first Fab has CDR sequences identified by SEQ ID Numbers: 055, 137, 257, 363, 480 and 574 and wherein the second Fab has CDR sequences identified by SEQ ID Numbers: 639, 640, 641, 642, 643 and 644.

Alternatively, another bispecific antibody according to the invention is expressed and purified having a first Fab that binds to the stem helix of a SARS-COV-2 and a second Fab that binds to the fusion peptide of SARS-COV-2, wherein the first Fab has CDR sequences identified by SEQ ID Numbers: 055, 137, 257, 363, 480 and 574 and wherein the second Fab has CDR sequences identified by SEQ ID Numbers: 645, 646, 647, 648, 649 and 650.

### 2. CHARACTERIZATION OF THE BISPECIFIC ANTIBODY

The purity and integrity of the bispecific are confirm using SDS-PAGE and Western blot analysis. The size and structure of the bispecific is assessed using techniques such as, size-exclusion chromatography and mass spectrometry.

### 3. BINDING AFFINITY ANALYSIS

An enzyme-linked immunosorbent assay (ELISA) is performed to confirm binding to each target antigen separately i.e. the stem helix of SARS-COV-2 and then separately the fusion peptide of SARS-COV-2.

Using surface plasmon resonance (SPR) or Biolayer Interferometry (BLI) determine the binding kinetics and affinity of each Fab region to SARS-COV-2 stem helix and then separately the fusion peptide of SARS-COV-2. SPR can also be used in competition assays.

### 4. BISPECIFIC BINDING COMPETITION ASSAY FOR THE STEM HELIX AND FUSION PEPTIDE OF SARS-COV-2 SPIKE PROTEIN

### 4.1. Materials and Reagents

Purified bispecific antibody according to the invention (bispecific antibody targeting both SARS-COV-2 stem helix and SARS-COV-2 fusion peptide).
Purified monospecific parent antibody 1, having CDR sequences identified by SEQ ID Numbers: 055, 137, 257, 363, 480 and 574, targeting only SARS-COV-2 stem helix.
Purified monospecific parent antibody 2, having CDR sequences identified by SEQ ID Numbers: 639, 640, 641, 642, 643 and 644, targeting only SARS-COV-2 fusion peptide may be used
Alternatively, purified monospecific parent antibody 2, having CDR sequences identified by SEQ ID Numbers: 645, 646, 647, 648, 649 and 650, targeting only SARS-COV-2 fusion peptide.
Purified label-conjugated monospecific parent antibody 1, having CDR sequences identified by SEQ ID Numbers: 055, 137, 257, 363, 480 and 574, targeting only SARS-CoV-2 stem helix.
Purified label-conjugated monospecific parent antibody 2, having CDR sequences identified by SEQ ID Numbers: 639, 640, 641, 642, 643 and 644, targeting only SARS-CoV-2 fusion peptide.
Alternatively, purified label-conjugated monospecific parent antibody 2, having CDR sequences identified by SEQ ID Numbers: 645, 646, 647, 648, 649 and 650, targeting only SARS-COV-2 fusion peptide.
Purified SARS-COV-2 virus spike protein having both the SARS-COV-2 stem helix epitope and SARS-COV-2 fusion peptide epitope.
PBS blocking buffer containing 1% bovine serum albumin (BSA)
PBS washing buffer containing Tween-20 0.05% (v/v).
Secondary direct detection antibody which has been conjugated to enzyme OR Substrate for enzyme-conjugated detection antibody.
96-well microplates and absorbance or fluorescence 96-well microplate reader.

### 4.2. Procedure

### Coating of the 96-Well Microplates

The pre-allocated wells of a microplate are coated with the purified SARS-COV-2 virus spike protein at a concentration optimized for antigen binding (optimised using, for example, either 0.1, 0.5, 1.0, 2.0, 3.0, 4.0, 5.0 and 10.0 μg/mL) in coating buffer and the covered microplates are incubated overnight at 4° C.

### Blocking of the Nonspecific Binding

The SARS-COV-2 virus spike-coated wells are wash three times with PBS washing buffer. Nonspecific binding is blocked by adding PBS blocking buffer to each well and the incubated for 1 hour at room temperature. The plates are then wash three times with PBS washing buffer.

### Blocking the Specific Binding

Into pre-allocated wells, specific binding is blocked by adding:
i. PBS blocking buffer and the anti-SARS-COV-2 stem helix monospecific antibody only at an optimised concentration for blocking the specific binding,
ii. PBS blocking buffer and the anti-SARS-COV-2 fusion peptide monospecific antibody only at an optimized concentration for blocking the specific binding,
iii. PBS blocking buffer and a mixture of both the anti-SARS-COV-2 stem helix monospecific antibody and the anti-SARS-COV-2 fusion peptide monospecific antibody at an optimised concentration for blocking the specific binding,
and the plates containing these wells are incubated for 1 hour at room temperature.

### Binding Competition Assay

Into pre-allocated wells, the following solutions are added:
Competition Solution 1: A solution of the label-conjugated monospecific antibody against SARS-COV-2 stem helix is prepared in PBS blocking buffer to an optimised concentration for the binding assay. The Competition Solution 1 is added to the pre-allocated coated wells described above and incubated for 2 hours at room temperature.
Competition Solution 2: A solution of the label-conjugated monospecific antibody against SARS-COV-2 fusion peptide is prepared in PBS blocking buffer to an optimised concentration for the binding assay. The Competition Solution 2 is added to the pre-allocated coated wells described above and incubated for 2 hours at room temperature.
Competition Solution 3: A solution of the label-conjugated bispecific antibody against both SARS-COV-2 stem helix and SARS-COV-2 fusion peptide is prepared in blocking buffer to an optimised concentration for the binding assay. The Competition Solution 3 is added to the pre-allocated coated wells described above and incubated for 2 hours at room temperature.

Note that assay optimization may be required for antibody concentrations, incubation times, and other parameters. Ensure that controls are included to validate the specificity of binding. Perform the assay in triplicate or as needed for statistical significance.

### 5. DETECTION

The 96-well microplates containing the assays referred to above are wash the three times with PBS washing buffer. If secondary antibody system is used, then add the appropriate detection antibody to each well and incubate for 1 hour at room temperature. If using an enzyme-conjugated detection antibody, then add the substrate according to the manufacturer's instructions.

### 6. MEASUREMENT

Measure absorbance or fluorescence in each well using a plate reader. Analyze the data, comparing the signals from wells with the bispecific antibody and monospecific antibodies.

### 7. DATA ANALYSIS

Calculate the competition between bispecific and monospecific antibodies for binding to the spike protein. Determine the relative binding affinity of the bispecific antibody to SARS-COV-2 stem helix and SARS-COV-2 fusion peptide epitopes.

### 8. CONCLUSION

The combination of in vitro binding assays, structural analysis, and in vivo validation provides comprehensive evidence supporting the bispecific nature of the antibody and its ability to bind to two different target antigens with the two distinct Fab regions.

1. An anti-coronavirus, preferably anti-SARS-COV-2 bispecific antibody or an antigen binding fragment thereof, having a first Fab capable of binding to the stem helix of a coronavirus, preferably SARS-COV-2 and a second Fab capable of binding to the fusion peptide of a coronavirus, preferably SARS-COV-2, wherein the first Fab comprises a sequence comprising any one or more of preferred SEQ ID Numbers: 055, 137, 257, 363, 480 or 574 and wherein the second Fab comprises a sequence comprising any one or more of preferred SEQ ID Numbers: 639, 640, 641, 642, 643 to 644.

2. A bispecific antibody or an antigen binding fragment thereof according to claim 1, comprising a first Fab capable of binding to the stem helix of a coronavirus, preferably SARS-COV-2, and a second Fab capable of binding to the fusion peptide of a coronavirus, preferably SARS-COV-2, wherein the first Fab comprises a sequence comprising any one or more of preferred SEQ ID Numbers: 055, 137, 257, 363, 480 and/or 574 and wherein the second Fab comprises a sequence comprising any one or more of preferred SEQ ID Numbers: 639, 640, 641, 642, 643 and/or 644.

3. A bispecific antibody or an antigen binding fragment thereof according to claim 1 or 2, comprising a first Fab specifically binding to the stem helix of a coronavirus, preferably SARS-COV-2, and a second Fab specifically binding to the fusion peptide of a coronavirus, preferably SARS-COV-2, characterized in that:
a.) the first Fab comprises a heavy chain variable region comprising as CDRs, a Heavy Chain CDR1 region of SEQ ID NO: 055, a Heavy Chain CDR2 region of SEQ ID NO: 137 and a Heavy Chain CDR3 region of SEQ ID NO: 257 and a light chain variable region comprising as CDRs a Light Chain CDR1 region of SEQ ID NO: 363, a Light Chain CDR2 region of SEQ ID NO: 480 and a Light Chain CDR3 region of SEQ ID NO: 574, and
b.) the second Fab comprises a heavy chain variable region comprising as CDRs, a Heavy Chain CDR1 region of SEQ ID NO: 639, a Heavy Chain CDR2 region of SEQ ID NO: 640 and a Heavy Chain CDR3 region of SEQ ID NO: 641 and a light chain variable region comprising as CDRs a Light Chain CDR1 region of SEQ ID NO: 642, a Light Chain CDR2 region of SEQ ID NO: 643 and a Light Chain CDR3 region of SEQ ID NO: 644.

4. A bispecific antibody or an antigen binding fragment thereof according to any of claims 1 to 3, comprising:
a) A light chain comprising VL-CL domains and a heavy chain comprising VH-CH1-CH2-CH3 domains of an antibody or an antigen binding fragment thereof, capable of binding to the stem helix of a coronavirus, preferably SARS-COV-2, and
b) A light chain comprising VL-CL domains and a heavy chain comprising VH-CH1-CH2-CH3 domains of an antibody or an antigen binding fragment thereof, capable of binding to the fusion peptide of a coronavirus, preferably SARS-COV-2, wherein the constant domains CL and CH1 from the antibody or an antigen binding fragment thereof that is capable of binding to the stem helix of a coronavirus, preferably SARS-COV-2, are replaced by each other.

5. A bispecific antibody or an antigen binding fragment thereof according to claim 4,
wherein the VH domain of the antibody or the antigen binding fragment thereof that is capable of binding to the stem helix of a coronavirus, preferably SARS-COV-2, comprises as CDRs, a Heavy Chain CDR1 region of SEQ ID NO: 055, a Heavy Chain CDR2 region of SEQ ID NO: 137 and a Heavy Chain CDR3 region of SEQ ID NO: 257 and the VL domain of the antibody or the antigen binding fragment thereof that is capable of binding to the stem helix of a coronavirus, preferably SARS-COV-2, comprises as CDRs a Light Chain CDR1 region of SEQ ID NO: 363, a Light Chain CDR2 region of SEQ ID NO: 480 and a Light Chain CDR3 region of SEQ ID NO: 574, and
wherein the VH domain of the antibody or the antigen binding fragment thereof that is capable of binding to the fusion peptide of a coronavirus, preferably SARS-CoV-2 comprises as CDRs, a Heavy Chain CDR1 region of SEQ ID NO: 639, a Heavy Chain CDR2 region of SEQ ID NO: 640 and a Heavy Chain CDR3 region of SEQ ID NO: 641 and the VL domain of the antibody or the antigen binding fragment thereof that is capable of binding to the fusion peptide of a coronavirus, preferably SARS-COV-2, comprises as CDRs a Light Chain CDR1 region of SEQ ID NO: 642, a Light Chain CDR2 region of SEQ ID NO: 643 and a Light Chain CDR3 region of SEQ ID NO: 644.

6. A bispecific antibody or an antigen binding fragment thereof according to any of claims 1 to 3, comprising:
a) A light chain comprising VL-CL domains and a heavy chain comprising VH-CH1-CH2-CH3 domains of an antibody, capable of binding to the stem helix of a coronavirus, preferably SARS-COV-2; and
b) A light chain comprising VL-CL domains and a heavy chain comprising VH-CH1-CH2-CH3 domains of an antibody, capable of binding to the fusion peptide of a coronavirus, preferably SARS-COV-2, wherein constant domains CL and CH1 from the antibody that is capable of binding to the fusion peptide of a coronavirus, preferably SARS-COV-2 are replaced by each other.

7. A bispecific antibody or an antigen binding fragment thereof according to claim 6,
wherein the VH domain of the antibody or the antigen binding fragment thereof that is capable of binding to the stem helix of a coronavirus, preferably SARS-COV-2, comprises as CDRs, a Heavy Chain CDR1 region of SEQ ID NO: 055, a Heavy Chain CDR2 region of SEQ ID NO: 137 and a Heavy Chain CDR3 region of SEQ ID NO: 257 and the VL domain of the antibody capable of binding to the stem helix of a coronavirus, preferably SARS-COV-2, comprises as CDRs a Light Chain CDR1 region of SEQ ID NO: 363, a Light Chain CDR2 region of SEQ ID NO: 480 and a Light Chain CDR3 region of SEQ ID NO: 574, and
wherein the VH domain of the antibody capable of binding to the fusion peptide of a coronavirus, preferably SARS-COV-2, comprises as CDRs, a Heavy Chain CDR1 region of SEQ ID NO: 639, a Heavy Chain CDR2 region of SEQ ID NO: 640 and a Heavy Chain CDR3 region of SEQ ID NO: 641 and the VL domain of the antibody capable of binding to the fusion peptide of a coronavirus, preferably SARS-COV-2, comprises as CDRs a Light Chain CDR1 region of SEQ ID NO: 642, a Light Chain CDR2 region of SEQ ID NO: 643 and a Light Chain CDR3 region of SEQ ID NO: 644.

8. A bispecific antibody or an antigen binding fragment thereof according to any of claims 1 to 3, comprising:
a) A light chain comprising VL-CL domains and a heavy chain comprising VH-CH1-CH2-CH3 domains of an antibody, capable of binding to the stem helix of a coronavirus, preferably SARS-COV-2; and
b) A light chain comprising VL-CL domains and a heavy chain comprising VH-CH1-CH2-CH3 domains of an antibody, capable of binding to the fusion peptide of a coronavirus, preferably SARS-COV-2, wherein domains VL-CL and VH-CH1 from the antibody that specifically binds to the fusion peptide of a coronavirus, preferably SARS-COV-2, are replaced by each other.

9. A bispecific antibody or an antigen binding fragment thereof according to any of claims 1 to 8, wherein the first Fab comprises a heavy chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 629.

10. A bispecific antibody or an antigen binding fragment thereof according to any of claims 1 to 8, wherein the second Fab comprises a heavy chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 673.

11. A bispecific antibody or an antigen binding fragment thereof according to any of claims 1 to 8, wherein the first Fab comprises a light chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 630.

12. A bispecific antibody or an antigen binding fragment thereof according to any of claims 1 to 8, wherein the second Fab comprises a light chain variable region having an amino acid sequence that is at least 90% identical, at least 91% identical, at least 92% identical, at least 93% identical, at least 94% identical, at least 95% identical, at least 96% identical, at least 97% identical, at least 98% identical, or at least 99% identical to the amino acid sequences set forth in SEQ ID NO: 674.

13. A bispecific antibody or an antigen binding fragment thereof according to any of claims 1 to 8, wherein the bispecific antibody or an antigen binding fragment thereof is selected from anyone of the group comprising a full-length antibody, a Fab, modified Fab, Fab′, modified Fab′, F(ab′) 2, Fv, single domain antibodies, scFv, scFv-Fc, bi, tri or tetra-valent antibodies, Bis-scFv, diabodies, triabodies, tetrabodies and epitope binding fragments thereof.

14. A method for the production of a bispecific antibody or an antigen binding fragment thereof according to any of claims 1 to 13, comprising a) culturing a host cell, comprising an expression vector, comprising a polynucleotide encoding a bispecific antibody or an antibody fragment according to any one of the preceding claims under conditions which permit the production of said bispecific antibody or an antigen binding fragment thereof, and b) isolating said bispecific antibody or an antigen binding fragment thereof.

15. A nucleic acid molecule comprising a nucleic acid sequence, wherein the nucleic acid sequence encodes the bispecific antibody or antigen binding fragment thereof, a heavy chain variable region and/or a light chain variable region of the bispecific antibody or an antigen binding fragment thereof according to any of claims 1 to 13, preferably wherein the nucleic acid molecule is an isolated nucleic acid molecule.

16. A nucleic acid molecule comprising a nucleic acid sequence, wherein the nucleic acid sequence encodes the bispecific antibody or antigen binding fragment thereof, a heavy chain variable region and/or light chain variable region of the bispecific antibody or an antigen binding fragment thereof according to any of claims 1 to 13 and claim 15, wherein the nucleic acid sequence comprises at least anyone of:
(i) a first nucleic acid sequence encoding the amino acid sequence as set forth in SEQ ID NO. 055, or a first nucleic acid sequence encoding the amino acid sequence having at most 1 or 2 amino acids different from SEQ ID NO. 055,
(ii) a second nucleic acid sequence encoding the amino acid sequence as set forth in SEQ ID NO. 137, or a second nucleic acid sequence encoding the amino acid sequence having at most 1, 2, 3, 4, or 5 amino acids different from SEQ ID NO. 137,
(iii) a third nucleic acid sequence encoding the amino acid sequence as set forth in SEQ ID NO. 257, or a third nucleic acid sequence encoding the amino acid sequence having at most 1, 2 or 3 amino acids different from SEQ ID NO. 257,
(iv) a fourth nucleic acid sequence encoding the amino acid sequence as set forth in SEQ ID NO. 363, or a fourth nucleic acid sequence encoding the amino acid sequence having at most 1, 2, 3 or 4 amino acids different from SEQ ID NO. 363,
(v) a fifth nucleic acid sequence encoding the amino acid sequence as set forth in SEQ ID NO. 480, or a fifth nucleic acid sequence encoding the amino acid sequence having at most 1 or 2 amino acids different from SEQ ID NO. 480,
(vi) a sixth nucleic acid sequence encoding the amino acid sequence as set forth in SEQ ID NO. 574, or a sixth nucleic acid sequence encoding the amino acid sequence having at most 1, 2, 3, or 4 amino acids different from SEQ ID NO. 574,
(vii) a seventh nucleic acid sequence encoding the amino acid sequence as set forth in SEQ ID NO. 639, or a seventh nucleic acid sequence encoding the amino acid sequence having at most 1 or 2 amino acids different from SEQ ID NO. 639,
(viii) an eighth nucleic acid sequence encoding the amino acid sequence as set forth in SEQ ID NO. 640, or an eighth nucleic acid sequence encoding the amino acid sequence having at most 1, 2, 3, 4, or 5 amino acids different from SEQ ID NO. 640,
(ix) a nineth nucleic acid sequence encoding the amino acid sequence as set forth in SEQ ID NO. 641, or a nineth nucleic acid sequence encoding the amino acid sequence having at most 1, 2, 3, or 4 amino acids different from SEQ ID NO. 641,
(x) a tenth nucleic acid sequence encoding the amino acid sequence as set forth in SEQ ID NO. 642, or a tenth nucleic acid sequence encoding the amino acid sequence having at most 1, 2, 3, or 4 amino acids different from SEQ ID NO. 642,
(xi) an eleventh nucleic acid sequence encoding the amino acid sequence as set forth in SEQ ID NO. 643, or an eleventh nucleic acid sequence encoding the amino acid sequence having at most 1 or 2 amino acids different from SEQ ID NO. 643, and
(xii) a twelfth nucleic acid sequence encoding the amino acid sequence as set forth in SEQ ID NO. 644, or a twelfth nucleic acid sequence encoding the amino acid sequence having at most 1, 2, or 3 amino acids different from SEQ ID NO. 644.

17. A bispecific antibody or an antigen binding fragment thereof according to any of claims 1 to 13 for use in a method of treating a coronavirus infection in a subject.

18. A composition comprising the bispecific antibody or an antigen binding fragment thereof according to any of claims 1 to 13.

19. A composition according to claim 18 further comprising a second medicament, for simultaneous, separate, or sequential administration.

20. A composition according to claim 19, wherein the second medicament comprises a second antibody or antigen binding fragment thereof.

21. A composition according to claim 19, wherein the second medicament comprises a bispecific antibody or bispecific antigen binding fragment thereof.

22. A composition according to claim 18, wherein the composition further comprises a pharmaceutically acceptable excipient or carrier.

23. A composition according to claim 18, for use in inhibiting and/or treating a coronavirus infection in a subject, preferably, a SARS-COV-1 or SARS-COV-2 infection in a subject, comprising an effective amount of the bispecific antibody or an antigen binding fragment thereof, nucleic acid molecule, or vector, and optionally a pharmaceutically acceptable excipient or carrier.

24. The composition of claim 18, for use as a medicament.

25. An inhaler device comprising the composition of claim 18.

26. A bispecific antibody or an antigen binding fragment thereof according to any of claims 1 to 13, wherein the bispecific antibody or an antigen binding fragment thereof is bivalent.

27. A bispecific antibody or an antigen binding fragment thereof according to any of claims 1 to 13, wherein the bispecific antibody or antigen-binding fragment thereof is capable of binding to the stem helix and/or the fusion peptide of at least one of an α-coronavirus, ß-coronavirus, γ-coronavirus, and δ-coronavirus, preferably at least an α-coronavirus and/or a ß-coronavirus.


SEQUENCE LISTING (683 sequences)

SEQ ID NO: 1 - protein, 15 aa, synthetic construct
GFTFSYFYLHWVRQA

SEQ ID NO: 2 - protein, 14 aa, synthetic construct
FTFSYFYLHWVRQA

SEQ ID NO: 3 - protein, 13 aa, synthetic construct
TFSYFYLHWVRQA

SEQ ID NO: 4 - protein, 12 aa, synthetic construct
FSYFYLHWVRQA

SEQ ID NO: 5 - protein, 11 aa, synthetic construct
SYFYLHWVRQA

SEQ ID NO: 6 - protein, 10 aa, synthetic construct
YFYLHWVRQA

SEQ ID NO: 7 - protein, 9 aa, synthetic construct
FYLHWVRQA

SEQ ID NO: 8 - protein, 8 aa, synthetic construct
YLHWVRQA

SEQ ID NO: 9 - protein, 7 aa, synthetic construct
LHWVRQA

SEQ ID NO: 10 - protein, 6 aa, synthetic construct
HWVRQA

SEQ ID NO: 11 - protein, 5 aa, synthetic construct
WVRQA

SEQ ID NO: 12 - protein, 14 aa, synthetic construct
GFTFSYFYLHWVRQ

SEQ ID NO: 13 - protein, 13 aa, synthetic construct
FTFSYFYLHWVRQ

SEQ ID NO: 14 - protein, 12 aa, synthetic construct
TFSYFYLHWVRQ

SEQ ID NO: 15 - protein, 11 aa, synthetic construct
FSYFYLHWVRQ

SEQ ID NO: 16 - protein, 10 aa, synthetic construct
SYFYLHWVRQ

SEQ ID NO: 17 - protein, 9 aa, synthetic construct
YFYLHWVRQ

SEQ ID NO: 18 - protein, 8 aa, synthetic construct
FYLHWVRQ

SEQ ID NO: 19 - protein, 7 aa, synthetic construct
YLHWVRQ

SEQ ID NO: 20 - protein, 6 aa, synthetic construct
LHWVRQ

SEQ ID NO: 21 - protein, 5 aa, synthetic construct
HWVRQ

SEQ ID NO: 22 - protein, 4 aa, synthetic construct
WVRQ

SEQ ID NO: 23 - protein, 13 aa, synthetic construct
GFTFSYFYLHWVR

SEQ ID NO: 24 - protein, 12 aa, synthetic construct
FTFSYFYLHWVR

SEQ ID NO: 25 - protein, 11 aa, synthetic construct
TFSYFYLHWVR

SEQ ID NO: 26 - protein, 10 aa, synthetic construct
FSYFYLHWVR

SEQ ID NO: 27 - protein, 9 aa, synthetic construct
SYFYLHWVR

SEQ ID NO: 28 - protein, 8 aa, synthetic construct
YFYLHWVR

SEQ ID NO: 29 - protein, 7 aa, synthetic construct
FYLHWVR

SEQ ID NO: 30 - protein, 6 aa, synthetic construct
YLHWVR

SEQ ID NO: 31 - protein, 5 aa, synthetic construct
LHWVR

SEQ ID NO: 32 - protein, 4 aa, synthetic construct
HWVR

SEQ ID NO: 33 - protein, 12 aa, synthetic construct
GFTFSYFYLHWV

SEQ ID NO: 34 - protein, 11 aa, synthetic construct
FTFSYFYLHWV

SEQ ID NO: 35 - protein, 10 aa, synthetic construct
TFSYFYLHWV

SEQ ID NO: 36 - protein, 9 aa, synthetic construct
FSYFYLHWV

SEQ ID NO: 37 - protein, 8 aa, synthetic construct
SYFYLHWV

SEQ ID NO: 38 - protein, 7 aa, synthetic construct
YFYLHWV

SEQ ID NO: 39 - protein, 6 aa, synthetic construct
FYLHWV

SEQ ID NO: 40 - protein, 5 aa, synthetic construct
YLHWV

SEQ ID NO: 41 - protein, 4 aa, synthetic construct
LHWV

SEQ ID NO: 42 - protein, 11 aa, synthetic construct
GFTFSYFYLHW

SEQ ID NO: 43 - protein, 10 aa, synthetic construct
FTFSYFYLHW

SEQ ID NO: 44 - protein, 9 aa, synthetic construct
TFSYFYLHW

SEQ ID NO: 45 - protein, 8 aa, synthetic construct
FSYFYLHW

SEQ ID NO: 46 - protein, 7 aa, synthetic construct
SYFYLHW

SEQ ID NO: 47 - protein, 6 aa, synthetic construct
YFYLHW

SEQ ID NO: 48 - protein, 5 aa, synthetic construct
FYLHW

SEQ ID NO: 49 - protein, 4 aa, synthetic construct
YLHW

SEQ ID NO: 50 - protein, 10 aa, synthetic construct
GFTFSYFYLH

SEQ ID NO: 51 - protein, 9 aa, synthetic construct
FTFSYFYLH

SEQ ID NO: 52 - protein, 8 aa, synthetic construct
TFSYFYLH

SEQ ID NO: 53 - protein, 7 aa, synthetic construct
FSYFYLH

SEQ ID NO: 54 - protein, 6 aa, synthetic construct
SYFYLH

SEQ ID NO: 55 - protein, 5 aa, synthetic construct
YFYLH

SEQ ID NO: 56 - protein, 4 aa, synthetic construct
FYLH

SEQ ID NO: 57 - protein, 9 aa, synthetic construct
GFTFSYFYL

SEQ ID NO: 58 - protein, 8 aa, synthetic construct
FTFSYFYL

SEQ ID NO: 59 - protein, 7 aa, synthetic construct
TFSYFYL

SEQ ID NO: 60 - protein, 6 aa, synthetic construct
FSYFYL

SEQ ID NO: 61 - protein, 5 aa, synthetic construct
SYFYL

SEQ ID NO: 62 - protein, 4 aa, synthetic construct
YFYL

SEQ ID NO: 63 - protein, 8 aa, synthetic construct
GFTFSYFY

SEQ ID NO: 64 - protein, 7 aa, synthetic construct
FTFSYFY

SEQ ID NO: 65 - protein, 6 aa, synthetic construct
TFSYFY

SEQ ID NO: 66 - protein, 5 aa, synthetic construct
FSYFY

SEQ ID NO: 67 - protein, 4 aa, synthetic construct
SYFY

SEQ ID NO: 68 - protein, 7 aa, synthetic construct
GFTFSYF

SEQ ID NO: 69 - protein, 6 aa, synthetic construct
FTFSYF

SEQ ID NO: 70 - protein, 5 aa, synthetic construct
TFSYF

SEQ ID NO: 71 - protein, 4 aa, synthetic construct
FSYF

SEQ ID NO: 72 - protein, 6 aa, synthetic construct
GFTFSY

SEQ ID NO: 73 - protein, 5 aa, synthetic construct
FTFSY

SEQ ID NO: 74 - protein, 4 aa, synthetic construct
TFSY

SEQ ID NO: 75 - protein, 5 aa, synthetic construct
GFTFS

SEQ ID NO: 76 - protein, 4 aa, synthetic construct
FTFS

SEQ ID NO: 77 - protein, 27 aa, synthetic construct
LEWMGIINPRGDGTRYAQKFQGRVTMT

SEQ ID NO: 78 - protein, 26 aa, synthetic construct
EWMGIINPRGDGTRYAQKFQGRVTMT

SEQ ID NO: 79 - protein, 25 aa, synthetic construct
WMGIINPRGDGTRYAQKFQGRVTMT

SEQ ID NO: 80 - protein, 24 aa, synthetic construct
MGIINPRGDGTRYAQKFQGRVTMT

SEQ ID NO: 81 - protein, 23 aa, synthetic construct
GIINPRGDGTRYAQKFQGRVTMT

SEQ ID NO: 82 - protein, 22 aa, synthetic construct
IINPRGDGTRYAQKFQGRVTMT

SEQ ID NO: 83 - protein, 21 aa, synthetic construct
INPRGDGTRYAQKFQGRVTMT

SEQ ID NO: 84 - protein, 20 aa, synthetic construct
NPRGDGTRYAQKFQGRVTMT

SEQ ID NO: 85 - protein, 19 aa, synthetic construct
PRGDGTRYAQKFQGRVTMT

SEQ ID NO: 86 - protein, 18 aa, synthetic construct
RGDGTRYAQKFQGRVTMT

SEQ ID NO: 87 - protein, 17 aa, synthetic construct
GDGTRYAQKFQGRVTMT

SEQ ID NO: 88 - protein, 26 aa, synthetic construct
LEWMGIINPRGDGTRYAQKFQGRVTM

SEQ ID NO: 89 - protein, 25 aa, synthetic construct
EWMGIINPRGDGTRYAQKFQGRVTM

SEQ ID NO: 90 - protein, 24 aa, synthetic construct
WMGIINPRGDGTRYAQKFQGRVTM

SEQ ID NO: 91 - protein, 23 aa, synthetic construct
MGIINPRGDGTRYAQKFQGRVTM

SEQ ID NO: 92 - protein, 22 aa, synthetic construct
GIINPRGDGTRYAQKFQGRVTM

SEQ ID NO: 93 - protein, 21 aa, synthetic construct
IINPRGDGTRYAQKFQGRVTM

SEQ ID NO: 94 - protein, 20 aa, synthetic construct
INPRGDGTRYAQKFQGRVTM

SEQ ID NO: 95 - protein, 19 aa, synthetic construct
NPRGDGTRYAQKFQGRVTM

SEQ ID NO: 96 - protein, 18 aa, synthetic construct
PRGDGTRYAQKFQGRVTM

SEQ ID NO: 97 - protein, 17 aa, synthetic construct
RGDGTRYAQKFQGRVTM

SEQ ID NO: 98 - protein, 16 aa, synthetic construct
GDGTRYAQKFQGRVTM

SEQ ID NO: 99 - protein, 25 aa, synthetic construct
LEWMGIINPRGDGTRYAQKFQGRVT

SEQ ID NO: 100 - protein, 24 aa, synthetic construct
EWMGIINPRGDGTRYAQKFQGRVT

SEQ ID NO: 101 - protein, 23 aa, synthetic construct
WMGIINPRGDGTRYAQKFQGRVT

SEQ ID NO: 102 - protein, 22 aa, synthetic construct
MGIINPRGDGTRYAQKFQGRVT

SEQ ID NO: 103 - protein, 21 aa, synthetic construct
GIINPRGDGTRYAQKFQGRVT

SEQ ID NO: 104 - protein, 20 aa, synthetic construct
IINPRGDGTRYAQKFQGRVT

SEQ ID NO: 105 - protein, 19 aa, synthetic construct
INPRGDGTRYAQKFQGRVT

SEQ ID NO: 106 - protein, 18 aa, synthetic construct
NPRGDGTRYAQKFQGRVT

SEQ ID NO: 107 - protein, 17 aa, synthetic construct
PRGDGTRYAQKFQGRVT

SEQ ID NO: 108 - protein, 16 aa, synthetic construct
RGDGTRYAQKFQGRVT

SEQ ID NO: 109 - protein, 15 aa, synthetic construct
GDGTRYAQKFQGRVT

SEQ ID NO: 110 - protein, 24 aa, synthetic construct
LEWMGIINPRGDGTRYAQKFQGRV

SEQ ID NO: 111 - protein, 23 aa, synthetic construct
EWMGIINPRGDGTRYAQKFQGRV

SEQ ID NO: 112 - protein, 22 aa, synthetic construct
WMGIINPRGDGTRYAQKFQGRV

SEQ ID NO: 113 - protein, 21 aa, synthetic construct
MGIINPRGDGTRYAQKFQGRV

SEQ ID NO: 114 - protein, 20 aa, synthetic construct
GIINPRGDGTRYAQKFQGRV

SEQ ID NO: 115 - protein, 19 aa, synthetic construct
IINPRGDGTRYAQKFQGRV

SEQ ID NO: 116 - protein, 18 aa, synthetic construct
INPRGDGTRYAQKFQGRV

SEQ ID NO: 117 - protein, 17 aa, synthetic construct
NPRGDGTRYAQKFQGRV

SEQ ID NO: 118 - protein, 16 aa, synthetic construct
PRGDGTRYAQKFQGRV

SEQ ID NO: 119 - protein, 15 aa, synthetic construct
RGDGTRYAQKFQGRV

SEQ ID NO: 120 - protein, 14 aa, synthetic construct
GDGTRYAQKFQGRV

SEQ ID NO: 121 - protein, 23 aa, synthetic construct
LEWMGIINPRGDGTRYAQKFQGR

SEQ ID NO: 122 - protein, 22 aa, synthetic construct
EWMGIINPRGDGTRYAQKFQGR

SEQ ID NO: 123 - protein, 21 aa, synthetic construct
WMGIINPRGDGTRYAQKFQGR

SEQ ID NO: 124 - protein, 20 aa, synthetic construct
MGIINPRGDGTRYAQKFQGR

SEQ ID NO: 125 - protein, 19 aa, synthetic construct
GIINPRGDGTRYAQKFQGR

SEQ ID NO: 126 - protein, 18 aa, synthetic construct
IINPRGDGTRYAQKFQGR

SEQ ID NO: 127 - protein, 17 aa, synthetic construct
INPRGDGTRYAQKFQGR

SEQ ID NO: 128 - protein, 16 aa, synthetic construct
NPRGDGTRYAQKFQGR

SEQ ID NO: 129 - protein, 15 aa, synthetic construct
PRGDGTRYAQKFQGR

SEQ ID NO: 130 - protein, 14 aa, synthetic construct
RGDGTRYAQKFQGR

SEQ ID NO: 131 - protein, 13 aa, synthetic construct
GDGTRYAQKFQGR

SEQ ID NO: 132 - protein, 22 aa, synthetic construct
LEWMGIINPRGDGTRYAQKFQG

SEQ ID NO: 133 - protein, 21 aa, synthetic construct
EWMGIINPRGDGTRYAQKFQG

SEQ ID NO: 134 - protein, 20 aa, synthetic construct
WMGIINPRGDGTRYAQKFQG

SEQ ID NO: 135 - protein, 19 aa, synthetic construct
MGIINPRGDGTRYAQKFQG

SEQ ID NO: 136 - protein, 18 aa, synthetic construct
GIINPRGDGTRYAQKFQG

SEQ ID NO: 137 - protein, 17 aa, synthetic construct
IINPRGDGTRYAQKFQG

SEQ ID NO: 138 - protein, 16 aa, synthetic construct
INPRGDGTRYAQKFQG

SEQ ID NO: 139 - protein, 15 aa, synthetic construct
NPRGDGTRYAQKFQG

SEQ ID NO: 140 - protein, 14 aa, synthetic construct
PRGDGTRYAQKFQG

SEQ ID NO: 141 - protein, 13 aa, synthetic construct
RGDGTRYAQKFQG

SEQ ID NO: 142 - protein, 12 aa, synthetic construct
GDGTRYAQKFQG

SEQ ID NO: 143 - protein, 21 aa, synthetic construct
LEWMGIINPRGDGTRYAQKFQ

SEQ ID NO: 144 - protein, 20 aa, synthetic construct
EWMGIINPRGDGTRYAQKFQ

SEQ ID NO: 145 - protein, 19 aa, synthetic construct
WMGIINPRGDGTRYAQKFQ

SEQ ID NO: 146 - protein, 18 aa, synthetic construct
MGIINPRGDGTRYAQKFQ

SEQ ID NO: 147 - protein, 17 aa, synthetic construct
GIINPRGDGTRYAQKFQ

SEQ ID NO: 148 - protein, 16 aa, synthetic construct
IINPRGDGTRYAQKFQ

SEQ ID NO: 149 - protein, 15 aa, synthetic construct
INPRGDGTRYAQKFQ

SEQ ID NO: 150 - protein, 14 aa, synthetic construct
NPRGDGTRYAQKFQ

SEQ ID NO: 151 - protein, 13 aa, synthetic construct
PRGDGTRYAQKFQ

SEQ ID NO: 152 - protein, 12 aa, synthetic construct
RGDGTRYAQKFQ

SEQ ID NO: 153 - protein, 11 aa, synthetic construct
GDGTRYAQKFQ

SEQ ID NO: 154 - protein, 20 aa, synthetic construct
LEWMGIINPRGDGTRYAQKF

SEQ ID NO: 155 - protein, 19 aa, synthetic construct
EWMGIINPRGDGTRYAQKF

SEQ ID NO: 156 - protein, 18 aa, synthetic construct
WMGIINPRGDGTRYAQKF

SEQ ID NO: 157 - protein, 17 aa, synthetic construct
MGIINPRGDGTRYAQKF

SEQ ID NO: 158 - protein, 16 aa, synthetic construct
GIINPRGDGTRYAQKF

SEQ ID NO: 159 - protein, 15 aa, synthetic construct
IINPRGDGTRYAQKF

SEQ ID NO: 160 - protein, 14 aa, synthetic construct
INPRGDGTRYAQKF

SEQ ID NO: 161 - protein, 13 aa, synthetic construct
NPRGDGTRYAQKF

SEQ ID NO: 162 - protein, 12 aa, synthetic construct
PRGDGTRYAQKF

SEQ ID NO: 163 - protein, 11 aa, synthetic construct
RGDGTRYAQKF

SEQ ID NO: 164 - protein, 10 aa, synthetic construct
GDGTRYAQKF

SEQ ID NO: 165 - protein, 19 aa, synthetic construct
LEWMGIINPRGDGTRYAQK

SEQ ID NO: 166 - protein, 18 aa, synthetic construct
EWMGIINPRGDGTRYAQK

SEQ ID NO: 167 - protein, 17 aa, synthetic construct
WMGIINPRGDGTRYAQK

SEQ ID NO: 168 - protein, 16 aa, synthetic construct
MGIINPRGDGTRYAQK

SEQ ID NO: 169 - protein, 15 aa, synthetic construct
GIINPRGDGTRYAQK

SEQ ID NO: 170 - protein, 14 aa, synthetic construct
IINPRGDGTRYAQK

SEQ ID NO: 171 - protein, 13 aa, synthetic construct
INPRGDGTRYAQK

SEQ ID NO: 172 - protein, 12 aa, synthetic construct
NPRGDGTRYAQK

SEQ ID NO: 173 - protein, 11 aa, synthetic construct
PRGDGTRYAQK

SEQ ID NO: 174 - protein, 10 aa, synthetic construct
RGDGTRYAQK

SEQ ID NO: 175 - protein, 9 aa, synthetic construct
GDGTRYAQK

SEQ ID NO: 176 - protein, 18 aa, synthetic construct
LEWMGIINPRGDGTRYAQ

SEQ ID NO: 177 - protein, 17 aa, synthetic construct
EWMGIINPRGDGTRYAQ

SEQ ID NO: 178 - protein, 16 aa, synthetic construct
WMGIINPRGDGTRYAQ

SEQ ID NO: 179 - protein, 15 aa, synthetic construct
MGIINPRGDGTRYAQ

SEQ ID NO: 180 - protein, 14 aa, synthetic construct
GIINPRGDGTRYAQ

SEQ ID NO: 181 - protein, 13 aa, synthetic construct
IINPRGDGTRYAQ

SEQ ID NO: 182 - protein, 12 aa, synthetic construct
INPRGDGTRYAQ

SEQ ID NO: 183 - protein, 11 aa, synthetic construct
NPRGDGTRYAQ

SEQ ID NO: 184 - protein, 10 aa, synthetic construct
PRGDGTRYAQ

SEQ ID NO: 185 - protein, 9 aa, synthetic construct
RGDGTRYAQ

SEQ ID NO: 186 - protein, 8 aa, synthetic construct
GDGTRYAQ

SEQ ID NO: 187 - protein, 17 aa, synthetic construct
LEWMGIINPRGDGTRYA

SEQ ID NO: 188 - protein, 16 aa, synthetic construct
EWMGIINPRGDGTRYA

SEQ ID NO: 189 - protein, 15 aa, synthetic construct
WMGIINPRGDGTRYA

SEQ ID NO: 190 - protein, 14 aa, synthetic construct
MGIINPRGDGTRYA

SEQ ID NO: 191 - protein, 13 aa, synthetic construct
GIINPRGDGTRYA

SEQ ID NO: 192 - protein, 12 aa, synthetic construct
IINPRGDGTRYA

SEQ ID NO: 193 - protein, 11 aa, synthetic construct
INPRGDGTRYA

SEQ ID NO: 194 - protein, 10 aa, synthetic construct
NPRGDGTRYA

SEQ ID NO: 195 - protein, 9 aa, synthetic construct
PRGDGTRYA

SEQ ID NO: 196 - protein, 8 aa, synthetic construct
RGDGTRYA

SEQ ID NO: 197 - protein, 19 aa, synthetic construct
YYCARGADHGAFDIWGQGT

SEQ ID NO: 198 - protein, 18 aa, synthetic construct
YCARGADHGAFDIWGQGT

SEQ ID NO: 199 - protein, 17 aa, synthetic construct
CARGADHGAFDIWGQGT

SEQ ID NO: 200 - protein, 16 aa, synthetic construct
ARGADHGAFDIWGQGT

SEQ ID NO: 201 - protein, 15 aa, synthetic construct
RGADHGAFDIWGQGT

SEQ ID NO: 202 - protein, 14 aa, synthetic construct
GADHGAFDIWGQGT

SEQ ID NO: 203 - protein, 13 aa, synthetic construct
ADHGAFDIWGQGT

SEQ ID NO: 204 - protein, 12 aa, synthetic construct
DHGAFDIWGQGT

SEQ ID NO: 205 - protein, 11 aa, synthetic construct
HGAFDIWGQGT

SEQ ID NO: 206 - protein, 10 aa, synthetic construct
GAFDIWGQGT

SEQ ID NO: 207 - protein, 9 aa, synthetic construct
AFDIWGQGT

SEQ ID NO: 208 - protein, 18 aa, synthetic construct
YYCARGADHGAFDIWGQG

SEQ ID NO: 209 - protein, 17 aa, synthetic construct
YCARGADHGAFDIWGQG

SEQ ID NO: 210 - protein, 16 aa, synthetic construct
CARGADHGAFDIWGQG

SEQ ID NO: 211 - protein, 15 aa, synthetic construct
ARGADHGAFDIWGQG

SEQ ID NO: 212 - protein, 14 aa, synthetic construct
RGADHGAFDIWGQG

SEQ ID NO: 213 - protein, 13 aa, synthetic construct
GADHGAFDIWGQG

SEQ ID NO: 214 - protein, 12 aa, synthetic construct
ADHGAFDIWGQG

SEQ ID NO: 215 - protein, 11 aa, synthetic construct
DHGAFDIWGQG

SEQ ID NO: 216 - protein, 10 aa, synthetic construct
HGAFDIWGQG

SEQ ID NO: 217 - protein, 9 aa, synthetic construct
GAFDIWGQG

SEQ ID NO: 218 - protein, 8 aa, synthetic construct
AFDIWGQG

SEQ ID NO: 219 - protein, 17 aa, synthetic construct
YYCARGADHGAFDIWGQ

SEQ ID NO: 220 - protein, 16 aa, synthetic construct
YCARGADHGAFDIWGQ

SEQ ID NO: 221 - protein, 15 aa, synthetic construct
CARGADHGAFDIWGQ

SEQ ID NO: 222 - protein, 14 aa, synthetic construct
ARGADHGAFDIWGQ

SEQ ID NO: 223 - protein, 13 aa, synthetic construct
RGADHGAFDIWGQ

SEQ ID NO: 224 - protein, 12 aa, synthetic construct
GADHGAFDIWGQ

SEQ ID NO: 225 - protein, 11 aa, synthetic construct
ADHGAFDIWGQ

SEQ ID NO: 226 - protein, 10 aa, synthetic construct
DHGAFDIWGQ

SEQ ID NO: 227 - protein, 9 aa, synthetic construct
HGAFDIWGQ

SEQ ID NO: 228 - protein, 8 aa, synthetic construct
GAFDIWGQ

SEQ ID NO: 229 - protein, 7 aa, synthetic construct
AFDIWGQ

SEQ ID NO: 230 - protein, 16 aa, synthetic construct
YYCARGADHGAFDIWG

SEQ ID NO: 231 - protein, 15 aa, synthetic construct
YCARGADHGAFDIWG

SEQ ID NO: 232 - protein, 14 aa, synthetic construct
CARGADHGAFDIWG

SEQ ID NO: 233 - protein, 13 aa, synthetic construct
ARGADHGAFDIWG

SEQ ID NO: 234 - protein, 12 aa, synthetic construct
RGADHGAFDIWG

SEQ ID NO: 235 - protein, 11 aa, synthetic construct
GADHGAFDIWG

SEQ ID NO: 236 - protein, 10 aa, synthetic construct
ADHGAFDIWG

SEQ ID NO: 237 - protein, 9 aa, synthetic construct
DHGAFDIWG

SEQ ID NO: 238 - protein, 8 aa, synthetic construct
HGAFDIWG

SEQ ID NO: 239 - protein, 7 aa, synthetic construct
GAFDIWG

SEQ ID NO: 240 - protein, 6 aa, synthetic construct
AFDIWG

SEQ ID NO: 241 - protein, 15 aa, synthetic construct
YYCARGADHGAFDIW

SEQ ID NO: 242 - protein, 14 aa, synthetic construct
YCARGADHGAFDIW

SEQ ID NO: 243 - protein, 13 aa, synthetic construct
CARGADHGAFDIW

SEQ ID NO: 244 - protein, 12 aa, synthetic construct
ARGADHGAFDIW

SEQ ID NO: 245 - protein, 11 aa, synthetic construct
RGADHGAFDIW

SEQ ID NO: 246 - protein, 10 aa, synthetic construct
GADHGAFDIW

SEQ ID NO: 247 - protein, 9 aa, synthetic construct
ADHGAFDIW

SEQ ID NO: 248 - protein, 8 aa, synthetic construct
DHGAFDIW

SEQ ID NO: 249 - protein, 7 aa, synthetic construct
HGAFDIW

SEQ ID NO: 250 - protein, 6 aa, synthetic construct
GAFDIW

SEQ ID NO: 251 - protein, 5 aa, synthetic construct
AFDIW

SEQ ID NO: 252 - protein, 14 aa, synthetic construct
YYCARGADHGAFDI

SEQ ID NO: 253 - protein, 13 aa, synthetic construct
YCARGADHGAFDI

SEQ ID NO: 254 - protein, 12 aa, synthetic construct
CARGADHGAFDI

SEQ ID NO: 255 - protein, 11 aa, synthetic construct
ARGADHGAFDI

SEQ ID NO: 256 - protein, 10 aa, synthetic construct
RGADHGAFDI

SEQ ID NO: 257 - protein, 9 aa, synthetic construct
GADHGAFDI

SEQ ID NO: 258 - protein, 8 aa, synthetic construct
ADHGAFDI

SEQ ID NO: 259 - protein, 7 aa, synthetic construct
DHGAFDI

SEQ ID NO: 260 - protein, 6 aa, synthetic construct
HGAFDI

SEQ ID NO: 261 - protein, 5 aa, synthetic construct
GAFDI

SEQ ID NO: 262 - protein, 4 aa, synthetic construct
AFDI

SEQ ID NO: 263 - protein, 13 aa, synthetic construct
YYCARGADHGAFD

SEQ ID NO: 264 - protein, 12 aa, synthetic construct
YCARGADHGAFD

SEQ ID NO: 265 - protein, 11 aa, synthetic construct
CARGADHGAFD

SEQ ID NO: 266 - protein, 10 aa, synthetic construct
ARGADHGAFD

SEQ ID NO: 267 - protein, 9 aa, synthetic construct
RGADHGAFD

SEQ ID NO: 268 - protein, 8 aa, synthetic construct
GADHGAFD

SEQ ID NO: 269 - protein, 7 aa, synthetic construct
ADHGAFD

SEQ ID NO: 270 - protein, 6 aa, synthetic construct
DHGAFD

SEQ ID NO: 271 - protein, 5 aa, synthetic construct
HGAFD

SEQ ID NO: 272 - protein, 4 aa, synthetic construct
GAFD

SEQ ID NO: 273 - protein, 12 aa, synthetic construct
YYCARGADHGAF

SEQ ID NO: 274 - protein, 11 aa, synthetic construct
YCARGADHGAF

SEQ ID NO: 275 - protein, 10 aa, synthetic construct
CARGADHGAF

SEQ ID NO: 276 - protein, 9 aa, synthetic construct
ARGADHGAF

SEQ ID NO: 277 - protein, 8 aa, synthetic construct
RGADHGAF

SEQ ID NO: 278 - protein, 7 aa, synthetic construct
GADHGAF

SEQ ID NO: 279 - protein, 6 aa, synthetic construct
ADHGAF

SEQ ID NO: 280 - protein, 5 aa, synthetic construct
DHGAF

SEQ ID NO: 281 - protein, 4 aa, synthetic construct
HGAF

SEQ ID NO: 282 - protein, 11 aa, synthetic construct
YYCARGADHGA

SEQ ID NO: 283 - protein, 10 aa, synthetic construct
YCARGADHGA

SEQ ID NO: 284 - protein, 9 aa, synthetic construct
CARGADHGA

SEQ ID NO: 285 - protein, 8 aa, synthetic construct
ARGADHGA

SEQ ID NO: 286 - protein, 7 aa, synthetic construct
RGADHGA

SEQ ID NO: 287 - protein, 6 aa, synthetic construct
GADHGA

SEQ ID NO: 288 - protein, 5 aa, synthetic construct
ADHGA

SEQ ID NO: 289 - protein, 4 aa, synthetic construct
DHGA

SEQ ID NO: 290 - protein, 10 aa, synthetic construct
YYCARGADHG

SEQ ID NO: 291 - protein, 9 aa, synthetic construct
YCARGADHG

SEQ ID NO: 292 - protein, 8 aa, synthetic construct
CARGADHG

SEQ ID NO: 293 - protein, 7 aa, synthetic construct
ARGADHG

SEQ ID NO: 294 - protein, 6 aa, synthetic construct
RGADHG

SEQ ID NO: 295 - protein, 5 aa, synthetic construct
GADHG

SEQ ID NO: 296 - protein, 4 aa, synthetic construct
ADHG

SEQ ID NO: 297 - protein, 9 aa, synthetic construct
YYCARGADH

SEQ ID NO: 298 - protein, 8 aa, synthetic construct
YCARGADH

SEQ ID NO: 299 - protein, 7 aa, synthetic construct
CARGADH

SEQ ID NO: 300 - protein, 6 aa, synthetic construct
ARGADH

SEQ ID NO: 301 - protein, 5 aa, synthetic construct
RGADH

SEQ ID NO: 302 - protein, 4 aa, synthetic construct
GADH

SEQ ID NO: 303 - protein, 22 aa, synthetic construct
ATLSCRASQSVRRNYFAWYQQK

SEQ ID NO: 304 - protein, 21 aa, synthetic construct
TLSCRASQSVRRNYFAWYQQK

SEQ ID NO: 305 - protein, 20 aa, synthetic construct
LSCRASQSVRRNYFAWYQQK

SEQ ID NO: 306 - protein, 19 aa, synthetic construct
SCRASQSVRRNYFAWYQQK

SEQ ID NO: 307 - protein, 18 aa, synthetic construct
CRASQSVRRNYFAWYQQK

SEQ ID NO: 308 - protein, 17 aa, synthetic construct
RASQSVRRNYFAWYQQK

SEQ ID NO: 309 - protein, 16 aa, synthetic construct
ASQSVRRNYFAWYQQK

SEQ ID NO: 310 - protein, 15 aa, synthetic construct
SQSVRRNYFAWYQQK

SEQ ID NO: 311 - protein, 14 aa, synthetic construct
QSVRRNYFAWYQQK

SEQ ID NO: 312 - protein, 13 aa, synthetic construct
SVRRNYFAWYQQK

SEQ ID NO: 313 - protein, 12 aa, synthetic construct
VRRNYFAWYQQK

SEQ ID NO: 314 - protein, 21 aa, synthetic construct
ATLSCRASQSVRRNYFAWYQQ

SEQ ID NO: 315 - protein, 20 aa, synthetic construct
TLSCRASQSVRRNYFAWYQQ

SEQ ID NO: 316 - protein, 19 aa, synthetic construct
LSCRASQSVRRNYFAWYQQ

SEQ ID NO: 317 - protein, 18 aa, synthetic construct
SCRASQSVRRNYFAWYQQ

SEQ ID NO: 318 - protein, 17 aa, synthetic construct
CRASQSVRRNYFAWYQQ

SEQ ID NO: 319 - protein, 16 aa, synthetic construct
RASQSVRRNYFAWYQQ

SEQ ID NO: 320 - protein, 15 aa, synthetic construct
ASQSVRRNYFAWYQQ

SEQ ID NO: 321 - protein, 14 aa, synthetic construct
SQSVRRNYFAWYQQ

SEQ ID NO: 322 - protein, 13 aa, synthetic construct
QSVRRNYFAWYQQ

SEQ ID NO: 323 - protein, 12 aa, synthetic construct
SVRRNYFAWYQQ

SEQ ID NO: 324 - protein, 11 aa, synthetic construct
VRRNYFAWYQQ

SEQ ID NO: 325 - protein, 20 aa, synthetic construct
ATLSCRASQSVRRNYFAWYQ

SEQ ID NO: 326 - protein, 19 aa, synthetic construct
TLSCRASQSVRRNYFAWYQ

SEQ ID NO: 327 - protein, 18 aa, synthetic construct
LSCRASQSVRRNYFAWYQ

SEQ ID NO: 328 - protein, 17 aa, synthetic construct
SCRASQSVRRNYFAWYQ

SEQ ID NO: 329 - protein, 16 aa, synthetic construct
CRASQSVRRNYFAWYQ

SEQ ID NO: 330 - protein, 15 aa, synthetic construct
RASQSVRRNYFAWYQ

SEQ ID NO: 331 - protein, 14 aa, synthetic construct
ASQSVRRNYFAWYQ

SEQ ID NO: 332 - protein, 13 aa, synthetic construct
SQSVRRNYFAWYQ

SEQ ID NO: 333 - protein, 12 aa, synthetic construct
QSVRRNYFAWYQ

SEQ ID NO: 334 - protein, 11 aa, synthetic construct
SVRRNYFAWYQ

SEQ ID NO: 335 - protein, 10 aa, synthetic construct
VRRNYFAWYQ

SEQ ID NO: 336 - protein, 19 aa, synthetic construct
ATLSCRASQSVRRNYFAWY

SEQ ID NO: 337 - protein, 18 aa, synthetic construct
TLSCRASQSVRRNYFAWY

SEQ ID NO: 338 - protein, 17 aa, synthetic construct
LSCRASQSVRRNYFAWY

SEQ ID NO: 339 - protein, 16 aa, synthetic construct
SCRASQSVRRNYFAWY

SEQ ID NO: 340 - protein, 15 aa, synthetic construct
CRASQSVRRNYFAWY

SEQ ID NO: 341 - protein, 14 aa, synthetic construct
RASQSVRRNYFAWY

SEQ ID NO: 342 - protein, 13 aa, synthetic construct
ASQSVRRNYFAWY

SEQ ID NO: 343 - protein, 12 aa, synthetic construct
SQSVRRNYFAWY

SEQ ID NO: 344 - protein, 11 aa, synthetic construct
QSVRRNYFAWY

SEQ ID NO: 345 - protein, 10 aa, synthetic construct
SVRRNYFAWY

SEQ ID NO: 346 - protein, 9 aa, synthetic construct
VRRNYFAWY

SEQ ID NO: 347 - protein, 18 aa, synthetic construct
ATLSCRASQSVRRNYFAW

SEQ ID NO: 348 - protein, 17 aa, synthetic construct
TLSCRASQSVRRNYFAW

SEQ ID NO: 349 - protein, 16 aa, synthetic construct
LSCRASQSVRRNYFAW

SEQ ID NO: 350 - protein, 15 aa, synthetic construct
SCRASQSVRRNYFAW

SEQ ID NO: 351 - protein, 14 aa, synthetic construct
CRASQSVRRNYFAW

SEQ ID NO: 352 - protein, 13 aa, synthetic construct
RASQSVRRNYFAW

SEQ ID NO: 353 - protein, 12 aa, synthetic construct
ASQSVRRNYFAW

SEQ ID NO: 354 - protein, 11 aa, synthetic construct
SQSVRRNYFAW

SEQ ID NO: 355 - protein, 10 aa, synthetic construct
QSVRRNYFAW

SEQ ID NO: 356 - protein, 9 aa, synthetic construct
SVRRNYFAW

SEQ ID NO: 357 - protein, 8 aa, synthetic construct
VRRNYFAW

SEQ ID NO: 358 - protein, 17 aa, synthetic construct
ATLSCRASQSVRRNYFA

SEQ ID NO: 359 - protein, 16 aa, synthetic construct
TLSCRASQSVRRNYFA

SEQ ID NO: 360 - protein, 15 aa, synthetic construct
LSCRASQSVRRNYFA

SEQ ID NO: 361 - protein, 14 aa, synthetic construct
SCRASQSVRRNYFA

SEQ ID NO: 362 - protein, 13 aa, synthetic construct
CRASQSVRRNYFA

SEQ ID NO: 363 - protein, 12 aa, synthetic construct
RASQSVRRNYFA

SEQ ID NO: 364 - protein, 11 aa, synthetic construct
ASQSVRRNYFA

SEQ ID NO: 365 - protein, 10 aa, synthetic construct
SQSVRRNYFA

SEQ ID NO: 366 - protein, 9 aa, synthetic construct
QSVRRNYFA

SEQ ID NO: 367 - protein, 8 aa, synthetic construct
SVRRNYFA

SEQ ID NO: 368 - protein, 7 aa, synthetic construct
VRRNYFA

SEQ ID NO: 369 - protein, 16 aa, synthetic construct
ATLSCRASQSVRRNYF

SEQ ID NO: 370 - protein, 15 aa, synthetic construct
TLSCRASQSVRRNYF

SEQ ID NO: 371 - protein, 14 aa, synthetic construct
LSCRASQSVRRNYF

SEQ ID NO: 372 - protein, 13 aa, synthetic construct
SCRASQSVRRNYF

SEQ ID NO: 373 - protein, 12 aa, synthetic construct
CRASQSVRRNYF

SEQ ID NO: 374 - protein, 11 aa, synthetic construct
RASQSVRRNYF

SEQ ID NO: 375 - protein, 10 aa, synthetic construct
ASQSVRRNYF

SEQ ID NO: 376 - protein, 9 aa, synthetic construct
SQSVRRNYF

SEQ ID NO: 377 - protein, 8 aa, synthetic construct
QSVRRNYF

SEQ ID NO: 378 - protein, 7 aa, synthetic construct
SVRRNYF

SEQ ID NO: 379 - protein, 6 aa, synthetic construct
VRRNYF

SEQ ID NO: 380 - protein, 15 aa, synthetic construct
ATLSCRASQSVRRNY

SEQ ID NO: 381 - protein, 14 aa, synthetic construct
TLSCRASQSVRRNY

SEQ ID NO: 382 - protein, 13 aa, synthetic construct
LSCRASQSVRRNY

SEQ ID NO: 383 - protein, 12 aa, synthetic construct
SCRASQSVRRNY

SEQ ID NO: 384 - protein, 11 aa, synthetic construct
CRASQSVRRNY

SEQ ID NO: 385 - protein, 10 aa, synthetic construct
RASQSVRRNY

SEQ ID NO: 386 - protein, 9 aa, synthetic construct
ASQSVRRNY

SEQ ID NO: 387 - protein, 8 aa, synthetic construct
SQSVRRNY

SEQ ID NO: 388 - protein, 7 aa, synthetic construct
QSVRRNY

SEQ ID NO: 389 - protein, 6 aa, synthetic construct
SVRRNY

SEQ ID NO: 390 - protein, 5 aa, synthetic construct
VRRNY

SEQ ID NO: 391 - protein, 14 aa, synthetic construct
ATLSCRASQSVRRN

SEQ ID NO: 392 - protein, 13 aa, synthetic construct
TLSCRASQSVRRN

SEQ ID NO: 393 - protein, 12 aa, synthetic construct
LSCRASQSVRRN

SEQ ID NO: 394 - protein, 11 aa, synthetic construct
SCRASQSVRRN

SEQ ID NO: 395 - protein, 10 aa, synthetic construct
CRASQSVRRN

SEQ ID NO: 396 - protein, 9 aa, synthetic construct
RASQSVRRN

SEQ ID NO: 397 - protein, 8 aa, synthetic construct
ASQSVRRN

SEQ ID NO: 398 - protein, 7 aa, synthetic construct
SQSVRRN

SEQ ID NO: 399 - protein, 6 aa, synthetic construct
QSVRRN

SEQ ID NO: 400 - protein, 5 aa, synthetic construct
SVRRN

SEQ ID NO: 401 - protein, 4 aa, synthetic construct
VRRN

SEQ ID NO: 402 - protein, 13 aa, synthetic construct
ATLSCRASQSVRR

SEQ ID NO: 403 - protein, 12 aa, synthetic construct
TLSCRASQSVRR

SEQ ID NO: 404 - protein, 11 aa, synthetic construct
LSCRASQSVRR

SEQ ID NO: 405 - protein, 10 aa, synthetic construct
SCRASQSVRR

SEQ ID NO: 406 - protein, 9 aa, synthetic construct
CRASQSVRR

SEQ ID NO: 407 - protein, 8 aa, synthetic construct
RASQSVRR

SEQ ID NO: 408 - protein, 7 aa, synthetic construct
ASQSVRR

SEQ ID NO: 409 - protein, 6 aa, synthetic construct
SQSVRR

SEQ ID NO: 410 - protein, 5 aa, synthetic construct
QSVRR

SEQ ID NO: 411 - protein, 4 aa, synthetic construct
SVRR

SEQ ID NO: 412 - protein, 12 aa, synthetic construct
ATLSCRASQSVR

SEQ ID NO: 413 - protein, 11 aa, synthetic construct
TLSCRASQSVR

SEQ ID NO: 414 - protein, 10 aa, synthetic construct
LSCRASQSVR

SEQ ID NO: 415 - protein, 9 aa, synthetic construct
SCRASQSVR

SEQ ID NO: 416 - protein, 8 aa, synthetic construct
CRASQSVR

SEQ ID NO: 417 - protein, 7 aa, synthetic construct
RASQSVR

SEQ ID NO: 418 - protein, 6 aa, synthetic construct
ASQSVR

SEQ ID NO: 419 - protein, 5 aa, synthetic construct
SQSVR

SEQ ID NO: 420 - protein, 4 aa, synthetic construct
QSVR

SEQ ID NO: 421 - protein, 17 aa, synthetic construct
RLLIYDASTRATGIPDR

SEQ ID NO: 422 - protein, 16 aa, synthetic construct
LLIYDASTRATGIPDR

SEQ ID NO: 423 - protein, 15 aa, synthetic construct
LIYDASTRATGIPDR

SEQ ID NO: 424 - protein, 14 aa, synthetic construct
IYDASTRATGIPDR

SEQ ID NO: 425 - protein, 13 aa, synthetic construct
YDASTRATGIPDR

SEQ ID NO: 426 - protein, 12 aa, synthetic construct
DASTRATGIPDR

SEQ ID NO: 427 - protein, 11 aa, synthetic construct
ASTRATGIPDR

SEQ ID NO: 428 - protein, 10 aa, synthetic construct
STRATGIPDR

SEQ ID NO: 429 - protein, 9 aa, synthetic construct
TRATGIPDR

SEQ ID NO: 430 - protein, 8 aa, synthetic construct
RATGIPDR

SEQ ID NO: 431 - protein, 7 aa, synthetic construct
ATGIPDR

SEQ ID NO: 432 - protein, 16 aa, synthetic construct
RLLIYDASTRATGIPD

SEQ ID NO: 433 - protein, 15 aa, synthetic construct
LLIYDASTRATGIPD

SEQ ID NO: 434 - protein, 14 aa, synthetic construct
LIYDASTRATGIPD

SEQ ID NO: 435 - protein, 13 aa, synthetic construct
IYDASTRATGIPD

SEQ ID NO: 436 - protein, 12 aa, synthetic construct
YDASTRATGIPD

SEQ ID NO: 437 - protein, 11 aa, synthetic construct
DASTRATGIPD

SEQ ID NO: 438 - protein, 10 aa, synthetic construct
ASTRATGIPD

SEQ ID NO: 439 - protein, 9 aa, synthetic construct
STRATGIPD

SEQ ID NO: 440 - protein, 8 aa, synthetic construct
TRATGIPD

SEQ ID NO: 441 - protein, 7 aa, synthetic construct
RATGIPD

SEQ ID NO: 442 - protein, 6 aa, synthetic construct
ATGIPD

SEQ ID NO: 443 - protein, 15 aa, synthetic construct
RLLIYDASTRATGIP

SEQ ID NO: 444 - protein, 14 aa, synthetic construct
LLIYDASTRATGIP

SEQ ID NO: 445 - protein, 13 aa, synthetic construct
LIYDASTRATGIP

SEQ ID NO: 446 - protein, 12 aa, synthetic construct
IYDASTRATGIP

SEQ ID NO: 447 - protein, 11 aa, synthetic construct
YDASTRATGIP

SEQ ID NO: 448 - protein, 10 aa, synthetic construct
DASTRATGIP

SEQ ID NO: 449 - protein, 9 aa, synthetic construct
ASTRATGIP

SEQ ID NO: 450 - protein, 8 aa, synthetic construct
STRATGIP

SEQ ID NO: 451 - protein, 7 aa, synthetic construct
TRATGIP

SEQ ID NO: 452 - protein, 6 aa, synthetic construct
RATGIP

SEQ ID NO: 453 - protein, 5 aa, synthetic construct
ATGIP

SEQ ID NO: 454 - protein, 14 aa, synthetic construct
RLLIYDASTRATGI

SEQ ID NO: 455 - protein, 13 aa, synthetic construct
LLIYDASTRATGI

SEQ ID NO: 456 - protein, 12 aa, synthetic construct
LIYDASTRATGI

SEQ ID NO: 457 - protein, 11 aa, synthetic construct
IYDASTRATGI

SEQ ID NO: 458 - protein, 10 aa, synthetic construct
YDASTRATGI

SEQ ID NO: 459 - protein, 9 aa, synthetic construct
DASTRATGI

SEQ ID NO: 460 - protein, 8 aa, synthetic construct
ASTRATGI

SEQ ID NO: 461 - protein, 7 aa, synthetic construct
STRATGI

SEQ ID NO: 462 - protein, 6 aa, synthetic construct
TRATGI

SEQ ID NO: 463 - protein, 5 aa, synthetic construct
RATGI

SEQ ID NO: 464 - protein, 4 aa, synthetic construct
ATGI

SEQ ID NO: 465 - protein, 13 aa, synthetic construct
RLLIYDASTRATG

SEQ ID NO: 466 - protein, 12 aa, synthetic construct
LLIYDASTRATG

SEQ ID NO: 467 - protein, 11 aa, synthetic construct
LIYDASTRATG

SEQ ID NO: 468 - protein, 10 aa, synthetic construct
IYDASTRATG

SEQ ID NO: 469 - protein, 9 aa, synthetic construct
YDASTRATG

SEQ ID NO: 470 - protein, 8 aa, synthetic construct
DASTRATG

SEQ ID NO: 471 - protein, 7 aa, synthetic construct
ASTRATG

SEQ ID NO: 472 - protein, 6 aa, synthetic construct
STRATG

SEQ ID NO: 473 - protein, 5 aa, synthetic construct
TRATG

SEQ ID NO: 474 - protein, 4 aa, synthetic construct
RATG

SEQ ID NO: 475 - protein, 12 aa, synthetic construct
RLLIYDASTRAT

SEQ ID NO: 476 - protein, 11 aa, synthetic construct
LLIYDASTRAT

SEQ ID NO: 477 - protein, 10 aa, synthetic construct
LIYDASTRAT

SEQ ID NO: 478 - protein, 9 aa, synthetic construct
IYDASTRAT

SEQ ID NO: 479 - protein, 8 aa, synthetic construct
YDASTRAT

SEQ ID NO: 480 - protein, 7 aa, synthetic construct
DASTRAT

SEQ ID NO: 481 - protein, 6 aa, synthetic construct
ASTRAT

SEQ ID NO: 482 - protein, 5 aa, synthetic construct
STRAT

SEQ ID NO: 483 - protein, 4 aa, synthetic construct
TRAT

SEQ ID NO: 484 - protein, 11 aa, synthetic construct
RLLIYDASTRA

SEQ ID NO: 485 - protein, 10 aa, synthetic construct
LLIYDASTRA

SEQ ID NO: 486 - protein, 9 aa, synthetic construct
LIYDASTRA

SEQ ID NO: 487 - protein, 8 aa, synthetic construct
IYDASTRA

SEQ ID NO: 488 - protein, 7 aa, synthetic construct
YDASTRA

SEQ ID NO: 489 - protein, 6 aa, synthetic construct
DASTRA

SEQ ID NO: 490 - protein, 5 aa, synthetic construct
ASTRA

SEQ ID NO: 491 - protein, 4 aa, synthetic construct
STRA

SEQ ID NO: 492 - protein, 10 aa, synthetic construct
RLLIYDASTR

SEQ ID NO: 493 - protein, 9 aa, synthetic construct
LLIYDASTR

SEQ ID NO: 494 - protein, 8 aa, synthetic construct
LIYDASTR

SEQ ID NO: 495 - protein, 7 aa, synthetic construct
IYDASTR

SEQ ID NO: 496 - protein, 6 aa, synthetic construct
YDASTR

SEQ ID NO: 497 - protein, 5 aa, synthetic construct
DASTR

SEQ ID NO: 498 - protein, 4 aa, synthetic construct
ASTR

SEQ ID NO: 499 - protein, 9 aa, synthetic construct
RLLIYDAST

SEQ ID NO: 500 - protein, 8 aa, synthetic construct
LLIYDAST

SEQ ID NO: 501 - protein, 7 aa, synthetic construct
LIYDAST

SEQ ID NO: 502 - protein, 6 aa, synthetic construct
IYDAST

SEQ ID NO: 503 - protein, 5 aa, synthetic construct
YDAST

SEQ ID NO: 504 - protein, 4 aa, synthetic construct
DAST

SEQ ID NO: 505 - protein, 8 aa, synthetic construct
RLLIYDAS

SEQ ID NO: 506 - protein, 7 aa, synthetic construct
LLIYDAS

SEQ ID NO: 507 - protein, 6 aa, synthetic construct
LIYDAS

SEQ ID NO: 508 - protein, 5 aa, synthetic construct
IYDAS

SEQ ID NO: 509 - protein, 4 aa, synthetic construct
YDAS

SEQ ID NO: 510 - protein, 7 aa, synthetic construct
RLLIYDA

SEQ ID NO: 511 - protein, 6 aa, synthetic construct
LLIYDA

SEQ ID NO: 512 - protein, 5 aa, synthetic construct
LIYDA

SEQ ID NO: 513 - protein, 4 aa, synthetic construct
IYDA

SEQ ID NO: 514 - protein, 21 aa, synthetic construct
AVYYCQQYDSSPPMYIFGQGT

SEQ ID NO: 515 - protein, 20 aa, synthetic construct
VYYCQQYDSSPPMYIFGQGT

SEQ ID NO: 516 - protein, 19 aa, synthetic construct
YYCQQYDSSPPMYIFGQGT

SEQ ID NO: 517 - protein, 18 aa, synthetic construct
YCQQYDSSPPMYIFGQGT

SEQ ID NO: 518 - protein, 17 aa, synthetic construct
CQQYDSSPPMYIFGQGT

SEQ ID NO: 519 - protein, 16 aa, synthetic construct
QQYDSSPPMYIFGQGT

SEQ ID NO: 520 - protein, 15 aa, synthetic construct
QYDSSPPMYIFGQGT

SEQ ID NO: 521 - protein, 14 aa, synthetic construct
YDSSPPMYIFGQGT

SEQ ID NO: 522 - protein, 13 aa, synthetic construct
DSSPPMYIFGQGT

SEQ ID NO: 523 - protein, 12 aa, synthetic construct
SSPPMYIFGQGT

SEQ ID NO: 524 - protein, 11 aa, synthetic construct
SPPMYIFGQGT

SEQ ID NO: 525 - protein, 20 aa, synthetic construct
AVYYCQQYDSSPPMYIFGQG

SEQ ID NO: 526 - protein, 19 aa, synthetic construct
VYYCQQYDSSPPMYIFGQG

SEQ ID NO: 527 - protein, 18 aa, synthetic construct
YYCQQYDSSPPMYIFGQG

SEQ ID NO: 528 - protein, 17 aa, synthetic construct
YCQQYDSSPPMYIFGQG

SEQ ID NO: 529 - protein, 16 aa, synthetic construct
CQQYDSSPPMYIFGQG

SEQ ID NO: 530 - protein, 15 aa, synthetic construct
QQYDSSPPMYIFGQG

SEQ ID NO: 531 - protein, 14 aa, synthetic construct
QYDSSPPMYIFGQG

SEQ ID NO: 532 - protein, 13 aa, synthetic construct
YDSSPPMYIFGQG

SEQ ID NO: 533 - protein, 12 aa, synthetic construct
DSSPPMYIFGQG

SEQ ID NO: 534 - protein, 11 aa, synthetic construct
SSPPMYIFGQG

SEQ ID NO: 535 - protein, 10 aa, synthetic construct
SPPMYIFGQG

SEQ ID NO: 536 - protein, 19 aa, synthetic construct
AVYYCQQYDSSPPMYIFGQ

SEQ ID NO: 537 - protein, 18 aa, synthetic construct
VYYCQQYDSSPPMYIFGQ

SEQ ID NO: 538 - protein, 17 aa, synthetic construct
YYCQQYDSSPPMYIFGQ

SEQ ID NO: 539 - protein, 16 aa, synthetic construct
YCQQYDSSPPMYIFGQ

SEQ ID NO: 540 - protein, 15 aa, synthetic construct
CQQYDSSPPMYIFGQ

SEQ ID NO: 541 - protein, 14 aa, synthetic construct
QQYDSSPPMYIFGQ

SEQ ID NO: 542 - protein, 13 aa, synthetic construct
QYDSSPPMYIFGQ

SEQ ID NO: 543 - protein, 12 aa, synthetic construct
YDSSPPMYIFGQ

SEQ ID NO: 544 - protein, 11 aa, synthetic construct
DSSPPMYIFGQ

SEQ ID NO: 545 - protein, 10 aa, synthetic construct
SSPPMYIFGQ

SEQ ID NO: 546 - protein, 9 aa, synthetic construct
SPPMYIFGQ

SEQ ID NO: 547 - protein, 18 aa, synthetic construct
AVYYCQQYDSSPPMYIFG

SEQ ID NO: 548 - protein, 17 aa, synthetic construct
VYYCQQYDSSPPMYIFG

SEQ ID NO: 549 - protein, 16 aa, synthetic construct
YYCQQYDSSPPMYIFG

SEQ ID NO: 550 - protein, 15 aa, synthetic construct
YCQQYDSSPPMYIFG

SEQ ID NO: 551 - protein, 14 aa, synthetic construct
CQQYDSSPPMYIFG

SEQ ID NO: 552 - protein, 13 aa, synthetic construct
QQYDSSPPMYIFG

SEQ ID NO: 553 - protein, 12 aa, synthetic construct
QYDSSPPMYIFG

SEQ ID NO: 554 - protein, 11 aa, synthetic construct
YDSSPPMYIFG

SEQ ID NO: 555 - protein, 10 aa, synthetic construct
DSSPPMYIFG

SEQ ID NO: 556 - protein, 9 aa, synthetic construct
SSPPMYIFG

SEQ ID NO: 557 - protein, 8 aa, synthetic construct
SPPMYIFG

SEQ ID NO: 558 - protein, 17 aa, synthetic construct
AVYYCQQYDSSPPMYIF

SEQ ID NO: 559 - protein, 16 aa, synthetic construct
VYYCQQYDSSPPMYIF

SEQ ID NO: 560 - protein, 15 aa, synthetic construct
YYCQQYDSSPPMYIF

SEQ ID NO: 561 - protein, 14 aa, synthetic construct
YCQQYDSSPPMYIF

SEQ ID NO: 562 - protein, 13 aa, synthetic construct
CQQYDSSPPMYIF

SEQ ID NO: 563 - protein, 12 aa, synthetic construct
QQYDSSPPMYIF

SEQ ID NO: 564 - protein, 11 aa, synthetic construct
QYDSSPPMYIF

SEQ ID NO: 565 - protein, 10 aa, synthetic construct
YDSSPPMYIF

SEQ ID NO: 566 - protein, 9 aa, synthetic construct
DSSPPMYIF

SEQ ID NO: 567 - protein, 8 aa, synthetic construct
SSPPMYIF

SEQ ID NO: 568 - protein, 7 aa, synthetic construct
SPPMYIF

SEQ ID NO: 569 - protein, 16 aa, synthetic construct
AVYYCQQYDSSPPMYI

SEQ ID NO: 570 - protein, 15 aa, synthetic construct
VYYCQQYDSSPPMYI

SEQ ID NO: 571 - protein, 14 aa, synthetic construct
YYCQQYDSSPPMYI

SEQ ID NO: 572 - protein, 13 aa, synthetic construct
YCQQYDSSPPMYI

SEQ ID NO: 573 - protein, 12 aa, synthetic construct
CQQYDSSPPMYI

SEQ ID NO: 574 - protein, 11 aa, synthetic construct
QQYDSSPPMYI

SEQ ID NO: 575 - protein, 10 aa, synthetic construct
QYDSSPPMYI

SEQ ID NO: 576 - protein, 9 aa, synthetic construct
YDSSPPMYI

SEQ ID NO: 577 - protein, 8 aa, synthetic construct
DSSPPMYI

SEQ ID NO: 578 - protein, 7 aa, synthetic construct
SSPPMYI

SEQ ID NO: 579 - protein, 6 aa, synthetic construct
SPPMYI

SEQ ID NO: 580 - protein, 15 aa, synthetic construct
AVYYCQQYDSSPPMY

SEQ ID NO: 581 - protein, 14 aa, synthetic construct
VYYCQQYDSSPPMY

SEQ ID NO: 582 - protein, 13 aa, synthetic construct
YYCQQYDSSPPMY

SEQ ID NO: 583 - protein, 12 aa, synthetic construct
YCQQYDSSPPMY

SEQ ID NO: 584 - protein, 11 aa, synthetic construct
CQQYDSSPPMY

SEQ ID NO: 585 - protein, 10 aa, synthetic construct
QQYDSSPPMY

SEQ ID NO: 586 - protein, 9 aa, synthetic construct
QYDSSPPMY

SEQ ID NO: 587 - protein, 8 aa, synthetic construct
YDSSPPMY

SEQ ID NO: 588 - protein, 7 aa, synthetic construct
DSSPPMY

SEQ ID NO: 589 - protein, 6 aa, synthetic construct
SSPPMY

SEQ ID NO: 590 - protein, 5 aa, synthetic construct
SPPMY

SEQ ID NO: 591 - protein, 14 aa, synthetic construct
AVYYCQQYDSSPPM

SEQ ID NO: 592 - protein, 13 aa, synthetic construct
VYYCQQYDSSPPM

SEQ ID NO: 593 - protein, 12 aa, synthetic construct
YYCQQYDSSPPM

SEQ ID NO: 594 - protein, 11 aa, synthetic construct
YCQQYDSSPPM

SEQ ID NO: 595 - protein, 10 aa, synthetic construct
CQQYDSSPPM

SEQ ID NO: 596 - protein, 9 aa, synthetic construct
QQYDSSPPM

SEQ ID NO: 597 - protein, 8 aa, synthetic construct
QYDSSPPM

SEQ ID NO: 598 - protein, 7 aa, synthetic construct
YDSSPPM

SEQ ID NO: 599 - protein, 6 aa, synthetic construct
DSSPPM

SEQ ID NO: 600 - protein, 5 aa, synthetic construct
SSPPM

SEQ ID NO: 601 - protein, 4 aa, synthetic construct
SPPM

SEQ ID NO: 602 - protein, 13 aa, synthetic construct
AVYYCQQYDSSPP

SEQ ID NO: 603 - protein, 12 aa, synthetic construct
VYYCQQYDSSPP

SEQ ID NO: 604 - protein, 11 aa, synthetic construct
YYCQQYDSSPP

SEQ ID NO: 605 - protein, 10 aa, synthetic construct
YCQQYDSSPP

SEQ ID NO: 606 - protein, 9 aa, synthetic construct
CQQYDSSPP

SEQ ID NO: 607 - protein, 8 aa, synthetic construct
QQYDSSPP

SEQ ID NO: 608 - protein, 7 aa, synthetic construct
QYDSSPP

SEQ ID NO: 609 - protein, 6 aa, synthetic construct
YDSSPP

SEQ ID NO: 610 - protein, 5 aa, synthetic construct
DSSPP

SEQ ID NO: 611 - protein, 4 aa, synthetic construct
SSPP

SEQ ID NO: 612 - protein, 12 aa, synthetic construct
AVYYCQQYDSSP

SEQ ID NO: 613 - protein, 11 aa, synthetic construct
VYYCQQYDSSP

SEQ ID NO: 614 - protein, 10 aa, synthetic construct
YYCQQYDSSP

SEQ ID NO: 615 - protein, 9 aa, synthetic construct
YCQQYDSSP

SEQ ID NO: 616 - protein, 8 aa, synthetic construct
CQQYDSSP

SEQ ID NO: 617 - protein, 7 aa, synthetic construct
QQYDSSP

SEQ ID NO: 618 - protein, 6 aa, synthetic construct
QYDSSP

SEQ ID NO: 619 - protein, 5 aa, synthetic construct
YDSSP

SEQ ID NO: 620 - protein, 4 aa, synthetic construct
DSSP

SEQ ID NO: 621 - protein, 11 aa, synthetic construct
AVYYCQQYDSS

SEQ ID NO: 622 - protein, 10 aa, synthetic construct
VYYCQQYDSS

SEQ ID NO: 623 - protein, 9 aa, synthetic construct
YYCQQYDSS

SEQ ID NO: 624 - protein, 8 aa, synthetic construct
YCQQYDSS

SEQ ID NO: 625 - protein, 7 aa, synthetic construct
CQQYDSS

SEQ ID NO: 626 - protein, 6 aa, synthetic construct
QQYDSS

SEQ ID NO: 627 - protein, 5 aa, synthetic construct
QYDSS

SEQ ID NO: 628 - protein, 4 aa, synthetic construct
YDSS

SEQ ID NO: 629 - protein, 118 aa, synthetic construct
QEQLVQSGAEVKKPGASVKVSCKSSGFTFSYFYLHWVRQAPGQGLEWMGIINPRGDGTRYAQKFQGRVTMTRDASTGTLYMELRSLRSEDTAVYYCARGADHGAFDIWGQGTMVTVSS

SEQ ID NO: 630 - protein, 110 aa, synthetic construct
EIVLTQSPGTLSLSPGERATLSCRASQSVRRNYFAWYQQKRGQAPRLLIYDASTRATGIPDRFSGSGSGTDFTLTISRLEPEDFAVYYCQQYDSSPPMYIFGQGTKLEIK

SEQ ID NO: 631 - protein, 30 aa, synthetic construct
QEQLVQSGAEVKKPGASVKVSCKSSGFTFS

SEQ ID NO: 632 - protein, 14 aa, synthetic construct
WVRQAPGQGLEWMG

SEQ ID NO: 633 - protein, 32 aa, synthetic construct
RVTMTRDASTGTLYMELRSLRSEDTAVYYCAR

SEQ ID NO: 634 - protein, 11 aa, synthetic construct
WGQGTMVTVSS

SEQ ID NO: 635 - protein, 23 aa, synthetic construct
EIVLTQSPGTLSLSPGERATLSC

SEQ ID NO: 636 - protein, 15 aa, synthetic construct
WYQQKRGQAPRLLIY

SEQ ID NO: 637 - protein, 32 aa, synthetic construct
GIPDRFSGSGSGTDFTLTISRLEPEDFAVYYC

SEQ ID NO: 638 - protein, 10 aa, synthetic construct
FGQGTKLEIK

SEQ ID NO: 639 - protein, 5 aa, synthetic construct
DYRIH

SEQ ID NO: 640 - protein, 17 aa, synthetic construct
RMNPKSGDTNFAQKFQG

SEQ ID NO: 641 - protein, 15 aa, synthetic construct
LLIVGGFDPLDDFEV

SEQ ID NO: 642 - protein, 14 aa, synthetic construct
SGTSSDVGGYNFVS

SEQ ID NO: 643 - protein, 7 aa, synthetic construct
EVTKRPS

SEQ ID NO: 644 - protein, 10 aa, synthetic construct
SSYGGTNNLL

SEQ ID NO: 645 - protein, 5 aa, synthetic construct
GYAMH

SEQ ID NO: 646 - protein, 17 aa, synthetic construct
VISRDARNKYYADSVKG

SEQ ID NO: 647 - protein, 17 aa, synthetic construct
LIIPGITEPGSPDALDI

SEQ ID NO: 648 - protein, 11 aa, synthetic construct
RASQDISKWLA

SEQ ID NO: 649 - protein, 7 aa, synthetic construct
AASSLQS

SEQ ID NO: 650 - protein, 11 aa, synthetic construct
QQASSFPWSIT

SEQ ID NO: 651 - protein, 5 aa, synthetic construct
SHYMH

SEQ ID NO: 652 - protein, 17 aa, synthetic construct
IINPSGSGTAYGQKFQG

SEQ ID NO: 653 - protein, 8 aa, synthetic construct
GSGGLFAY

SEQ ID NO: 654 - protein, 12 aa, synthetic construct
RASQIVRSNYLA

SEQ ID NO: 655 - protein, 7 aa, synthetic construct
GASSRAT

SEQ ID NO: 656 - protein, 11 aa, synthetic construct
LQYDSSPPTYI

SEQ ID NO: 657 - protein, 5 aa, synthetic construct
SYYMH

SEQ ID NO: 658 - protein, 17 aa, synthetic construct
LITPSGDDTYYAQRFQG

SEQ ID NO: 659 - protein, 9 aa, synthetic construct
MSRAGGFDV

SEQ ID NO: 660 - protein, 12 aa, synthetic construct
RASQSITGRYLA

SEQ ID NO: 661 - protein, 7 aa, synthetic construct
GESSRVT

SEQ ID NO: 662 - protein, 11 aa, synthetic construct
QHFASSPPTYT

SEQ ID NO: 663 - protein, 452 aa, synthetic construct
QEQLVQSGAEVKKPGASVKVSCKSSGFTFSYFYLHWVRQAPGQGLEWMGIINPRGDGTRYAQKFQGRVTMTRDASTGTLYMELRSLRSEDTAVYYCARGADHGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 664 - protein, 215 aa, synthetic construct
EIVLTQSPGTLSLSPGERATLSCRASQSVRRNYFAWYQQKRGQAPRLLIYDASTRATGIPDRFSGSGSGTDFTLTISRLEPEDFAVYYCQQYDSSPPMYIFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 665 - protein, 454 aa, synthetic construct
QMQLMQSGAEVKKPGASVTVSCKASGDTFSDYRIHWVRQAPGQGLEWMGRMNPKSGDTNFAQKFQGRVTMTRDMSINTAYMTLSGLTFDDTALYYCASLLIVGGFDPLDDFEVWGQGTMVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 666 - protein, 216 aa, synthetic construct
QSALTQPPSASGSPGQSVTISCSGTSSDVGGYNFVSWYQHHPGKAPKILIYEVTKRPSGVPDRFSGSKSGNTASLTVSGLQAEDEADYYCSSYGGTNNLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 667 - protein, 448 aa, synthetic construct
QEQLVQSGAEVKKPGASVKVSCKSSGFTFSYFYLHWVRQAPGQGLEWMGIINPRGDGTRYAQKFQGRVTMTRDASTGTLYMELRSLRSEDTAVYYCARGADHGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 668 - protein, 217 aa, synthetic construct
EIVLTQSPGTLSLSPGERATLSCRASQSVRRNYFAWYQQKRGQAPRLLIYDASTRATGIPDRFSGSGSGTDFTLTISRLEPEDFAVYYCQQYDSSPPMYIFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 669 - protein, 456 aa, synthetic construct
QMQLMQSGAEVKKPGASVTVSCKASGDTFSDYRIHWVRQAPGQGLEWMGRMNPKSGDTNFAQKFQGRVTMTRDMSINTAYMTLSGLTFDDTALYYCASLLIVGGFDPLDDFEVWGQGTMVTISGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 670 - protein, 215 aa, synthetic construct
QSALTQPPSASGSPGQSVTISCSGTSSDVGGYNFVSWYQHHPGKAPKILIYEVTKRPSGVPDRFSGSKSGNTASLTVSGLQAEDEADYYCSSYGGTNNLLFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 671 - protein, 443 aa, synthetic construct
QSALTQPPSASGSPGQSVTISCSGTSSDVGGYNFVSWYQHHPGKAPKILIYEVTKRPSGVPDRFSGSKSGNTASLTVSGLQAEDEADYYCSSYGGTNNLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 672 - protein, 227 aa, synthetic construct
QMQLMQSGAEVKKPGASVTVSCKASGDTFSDYRIHWVRQAPGQGLEWMGRMNPKSGDTNFAQKFQGRVTMTRDMSINTAYMTLSGLTFDDTALYYCASLLIVGGFDPLDDFEVWGQGTMVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 673 - protein, 124 aa, synthetic construct
QMQLMQSGAEVKKPGASVTVSCKASGDTFSDYRIHWVRQAPGQGLEWMGRMNPKSGDTNFAQKFQGRVTMTRDMSINTAYMTLSGLTFDDTALYYCASLLIVGGFDPLDDFEVWGQGTMVTISS

SEQ ID NO: 674 - protein, 110 aa, synthetic construct
QSALTQPPSASGSPGQSVTISCSGTSSDVGGYNFVSWYQHHPGKAPKILIYEVTKRPSGVPDRFSGSKSGNTASLTVSGLQAEDEADYYCSSYGGTNNLLFGGGTKLTVL

SEQ ID NO: 675 - protein, 126 aa, synthetic construct [sequence omitted]

SEQ ID NO: 676 - protein, 109 aa, synthetic construct [sequence omitted]

SEQ ID NO: 677 - protein, 21 aa, HCoV-229E
VPEYIDVNKTLQELSYKLPNY

SEQ ID NO: 678 - protein, 21 aa, HCoV-NL63
IPDYVDVNKTLQEFAQNLPKY

SEQ ID NO: 679 - protein, 21 aa, HCoV-HKU1
IPNLSDFEAELSLWFKNHTSI

SEQ ID NO: 680 - protein, 21 aa, HCoV-OC43
IPNLPDFKEELDQWFKNQTSV

SEQ ID NO: 681 - protein, 21 aa, SARS-CoV-1
QPELDSFKEELDKYFKNHTSP

SEQ ID NO: 682 - protein, 21 aa, SARS-CoV-2
QPELDSFKEELDKYFKNHTSP

SEQ ID NO: 683 - protein, 23 aa, MERS-CoV
LGNSTGIDFQDELDEFFKNVSTS